(19) United States
(12) Patent Application Publication
(10) Pub. No.: US 2025/0051848 A1
(43) Pub. Date: Feb. 13, 2025

(54) HIGH-ACCURACY PREDICTION OF COLORECTAL CANCER CHEMOTHERAPY EFFICACY USING MACHINE LEARNING APPLIED TO GENE EXPRESSION DATA

(71) Applicant: George Mason University, Fairfax, VA (US)

(72) Inventors: Mohsin Saleet Jafri, Potomac, MD (US); Soukaina Amniouel, Fairfax, VA (US)

(21) Appl. No.: 18/447,751

(22) Filed: Aug. 10, 2023

(51) Int. Cl. C12Q 1/6886; C12Q 1/686

(52) U.S. Cl. CPC C12Q 1/6886; C12Q 1/686; C12Q 2600/158; G01N 2800/52

(57) ABSTRACT

The present disclosure generally relates to gene expression profiling of tissue samples obtained from colorectal cancer patients who are candidates for chemotherapy treatment. More specifically, the disclosure provides methods based on characterization of gene expression which allow a physician to predict whether a patient is likely to respond well to treatment with a chemotherapeutic reagent.

### TECHNICAL FIELD

The present disclosure generally relates to gene expression profiling of tissue samples obtained from colorectal cancer patients who are candidates for chemotherapy treatment. More specifically, the disclosure provides methods based on characterization of gene expression which allow a physician to predict whether a patient is likely to respond well to treatment with a chemotherapeutic reagent.

### BACKGROUND

Colorectal cancer (CRC) is the predominant cancer affecting the gastrointestinal tract and is recognized as the third most widespread form of cancer in both males and females. Despite substantial endeavors in preventive screening and advancements in treatment choices, CRC continues to be a major contributor to cancer-related morbidity and mortality [1].

The chemotherapy backbone for first-line treatment of metastatic disease is typically either FOLFOX (FOL=Leucovorin Calcium (Folinic Acid), F=Fluorouracil and OX=Oxaliplatin) or FOLFIRI (FOL=Leucovorin Calcium (Folinic Acid), F=Fluorouracil and IRI=Irinotecan Hydrochloride) [7, 8]. Despite the progress made in cytotoxic therapy, the development of resistance to chemotherapy remains a significant obstacle in the long-term management of incurable metastatic disease. Eventually, this resistance contributes to mortality, as cancer cells find ways to adapt and become tolerant to pharmaceutical treatments [12, 13]. There is currently a lack of studies investigating predictive biomarkers that can effectively differentiate between the use of FOLFOX or FOLFIRI cytotoxic agents in the treatment of patients.

### SUMMARY

The present disclosure relates to studies of gene expression levels in tissue samples obtained from patients with different stages of colorectal cancer (CRC) who responded or did not respond to treatment with FOLFOX or FOLFIRI. As described herein, prognostic mRNAs, referred to herein as “biomarkers”, have been identified wherein the expression levels of said biomarkers correlated to the likelihood of responding to FOLFOX or FOLFIRI chemotherapy. All biomarker gene symbols, as used herein, are those adopted by the HUGO Gene Nomenclature Committee. Amino acid and nucleic acid sequences corresponding to each of the listed genes are publicly available.

In one embodiment, the disclosure provides a method for predicting the likelihood that patients at all stages of CRC, who are candidates for treatment with FOLFOX, will respond to such treatment, comprising determining the expression level of one or more biomarker transcripts, or their expression product, in a CRC patient sample, wherein the biomarker transcript is the transcript of one or more genes selected from the group consisting of: GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2, and EPHB3. (FIG. 21)

In one embodiment, the disclosure provides a method for predicting the likelihood that patients at early stages of CRC, who are candidates for treatment with FOLFOX, will respond to such treatment, comprising determining the expression level of one or more biomarker RNA transcripts, or their expression product, in a CRC patient sample, wherein the biomarker transcript is the transcript of one or more genes selected from the group consisting of: PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2. (FIG. 22)

In one embodiment, the disclosure provides a method for predicting the likelihood that patients with metastatic CRC, who are candidates for treatment with FOLFOX, will respond to such treatment, comprising determining the expression level of one or more biomarker RNA transcripts, or their expression product, in a CRC patient sample, wherein the biomarker transcript is the transcript of one or more genes selected from the group consisting of EEF1D, HSD17B2, ADH1B, RPS10, CA2, RPS1,1 ADAMDEC1, AKRIB10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL3RA2, NME7, REEP1, TACSTD2, EDNRB, NR5A2, P2RYJ4, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI. (FIG. 23)

In one embodiment, the disclosure provides a method for predicting the likelihood that patients at all stages of CRC, who are candidates for treatment with FOLFIRI, will respond to such treatment, comprising determining the expression level of one or more biomarker RNA transcripts, or their expression product, in a CRC patient sample, wherein the biomarker transcript is the transcript of one or more genes selected from the group consisting of: AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1. (FIG. 24)

In one embodiment, the disclosure provides a method for predicting the likelihood that patients at early stages of CRC, who are candidates for treatment with FOLFIRI, will respond to such treatment, comprising determining the expression level of one or more biomarker RNA transcripts, or their expression product, in a CRC patient sample wherein the biomarker transcript is the transcript of one or more genes selected from the group consisting of: including CCN4, ABCC13, AKAP12, GLT8D2, LUM, CTHRC1, FLRT3, SFRP2, COL1A2, MIR100HG, and PCDH7. (FIG. 25)

In one embodiment, the disclosure provides a method for predicting the likelihood that patients with metastatic CRC, who are candidates for treatment with FOLFIRI, will respond to such treatment, comprising determining the expression level of one or more biomarker RNA transcripts, or their expression product, in a CRC patient sample wherein the biomarker transcript is the transcript of one or more genes selected from the group consisting of PTGR2, P115, RAB3IP, USP48, PCDHB16, DDR2, PAPPA, FXYD5, ZNF300, FBXO32, ZNFJ17, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBX06. (FIG. 26)

The expression levels of one or more biomarker mRNA transcripts, or their protein products, can be determined by methods known in the art. Methods for detecting expression of the biomarker genes disclosed herein include methods based on hybridization analysis of polynucleotides, methods based on sequencing of polynucleotides, immunohistochemistry methods, and proteomics-based methods. The methods generally detect expression products (e.g., mRNA or encoded proteins) of the biomarker genes. In preferred embodiments, PCR-based methods, such as reverse transcription PCR (RT-PCR), and array-based methods are used.

In one aspect, microarrays are provided comprising one or more biomarker genes that demonstrate altered expression following exposure to FOLFOX or FOLFIRI. In an embodiment, the microarray may comprise one or more probes representative of the biomarker genes disclosed herein wherein the expression levels of said genes correlate to a CFC patient's likelihood of responding to FOLFOX or FOLFIRI chemotherapy. In another aspect, methods are provided for using said microarrays to provide a patient's prognosis for responding to FOLFOX or FOLFIRI chemotherapy through the generation of an expression profile indicating that the patient is a candidate for treatment with FOLFOX or FOLFIRI chemotherapy.

In an embodiment a method is provided for determining a patient's prognosis for responding to FOLFOX or FOLFIRI chemotherapy comprising the steps of: (i) providing a nucleic acid probe comprising a nucleotide sequence having at least 10, at least 15, at least 25 or at least 40 consecutive nucleotides complementary to the one or more biomarker RNA transcripts disclosed herein, wherein the expression levels of the one or more biomarker RNAs correlates to a CFC patient's likelihood of responding to FOLFOX or FOLFIRI chemotherapy; (ii) contacting the nucleic acid probe under stringent conditions with the mRNA of a patient's tissue sample; and (iii) detecting the amount of hybridization, wherein comparison of the amount of hybridization with the RNA of the patient's test sample to a threshold amount of hybridization of determined by analysis of many test samples, i.e., a non-responder and/or a responder sample, is indicative of the patient's prognosis for responding to FOLFOX or FOLFIRI chemotherapy.

The present disclosure provides a method of preparing a prognostic profile for a CRC patient, comprising the steps of: (i) subjecting a patient's sample containing biomarker mRNA to gene expression analysis; (ii) determining the expression level of one or more of the biomarker mRNAs disclosed herein wherein the expression level is compared to control levels of expression determined for responder and non-responder samples; and (iii) creating a report summarizing the data obtained by said gene expression analysis.

The present disclosure provides a kit for identifying the expression levels of one or more of the biomarker mRNAs disclosed herein. Said kit comprises a probe/primer for detecting the level of one or more biomarker RNAs in a sample derived from a patient. In certain embodiments, the kit may further include instructions for using the kit, solutions for suspending or fixing cells derived from the sample, detectable tags or labels, and solutions for lysing cells. In some instances, the kit may contain solutions and reagents for detecting the protein products of the biomarker mRNAs.

### BRIEF DESCRIPTION OF THE FIGURES

FIG. 1A-B. A multi-stage analysis methodology was applied to CRC patients from all stages who received (FIG. 1A) FOLFOX or (FIG. 1B) FOLFIRI chemotherapy. Gene expression profiling datasets of human colorectal tissues were collected from the NCBI-GEO database. The datasets were analyzed using the robust multi-array average method in R to identify differentially expressed genes (DEGs). Feature selection methods were performed using LASSO and varSelRF methods to identify gene signatures related to each chemotherapy drug (i.e., FOLFOX or FOLFIRI). The performance of the machine learning models was evaluated using random forest and support vector machine algorithms. Functional enrichment analysis of the gene signatures was performed to identify significantly enriched pathways and Gene Ontology (GO) terms. Protein-protein interaction networks were reconstructed around gene signatures.

FIG. 2A-D. Construction of LASSO model for patients with all stages of CRC who received FOLFOX therapy. (FIG. 2A) Ten-fold cross-validation for tuning parameter selection in the LASSO model. (FIG. 2B) LASSO coefficient profiles of the training set. (FIG. 2C) The prediction score of the classifier was higher in responder than non-responder samples in the training set. (FIG. 2D) The prediction score of the classifier was higher in responder than non-responder samples in the validation set.

FIG. 3A-D GO analyses of gene signatures associated with all stages of CRC patients who received FOLFOX chemotherapy. GO includes three categories: Biological Process, Molecular Function, and Cellular Component. (FIG. 3A) represents biological process that genes signatures are involved in; (FIG. 3B) Molecular function that the gene signatures are enriched in; (FIG. 3C) the cellular components that the gene signatures are correlated with. (FIG. 3D) represents the KEGG pathways associated with gene signatures identified in CRC patients who received FOLFOX chemotherapy.

FIG. 4A-D. Construction of LASSO model. (FIG. 4A) Ten-fold cross-validation for tuning parameter selection in the LASSO model. (FIG. 4B) LASSO coefficient profiles of the training set. (FIG. 4C) The prediction score of the classifier was higher in responder than non-responder samples in the training set. (FIG. 4D) The prediction score of the classifier was higher in responder than non-responder samples in the validation set.

FIG. 5A-D. GO analyses of gene signatures associated with early-stage CRC patients who received FOLFOX chemotherapy. GO includes three categories: Biological Process, Molecular Function, and Cellular Component. (FIG. 5A) represents a biological process that genes signatures are involved in; (FIG. 5B) Molecular function that the gene signatures are enriched in; (FIG. 5C) the cellular components that the gene signatures are correlated with. (FIG. 5D) represents the KEGG pathways associated with gene signatures identified in CRC patients who received FOLFOX chemotherapy.

FIG. 6A-D. Construction of LASSO model. (FIG. 6A) Ten-fold cross-validation for tuning parameter selection in the LASSO model. (FIG. 6B) LASSO coefficient profiles of the training set. (FIG. 6C) The prediction score of the classifier was higher in responder than non-responder samples in the training set. (FIG. 6D) The prediction score of the classifier was higher in responder than non-responder samples in the validation set.

The FIG. 7A-D. GO analyses of gene signatures associated with metastatic CRC patients who received FOLFOX chemotherapy. GO includes three categories: Biological Process, Molecular Function, and Cellular Component. (FIG. 7A) represents biological process that the gene signatures are involved in; (FIG. 7B) Molecular function that the gene signatures are enriched in; (FIG. 7C) the cellular components that the gene signatures are correlated with. (FIG. 7D) represents the KEGG pathways associated with gene signatures identified in metastatic CRC patients who received FOLFOX chemotherapy.

FIG. 8A-D. Construction of LASSO model. (FIG. 8A) Ten-fold cross-validation for tuning parameter selection in the LASSO model. (FIG. 8B) LASSO coefficient profiles of the training set. (FIG. 8C) The prediction score of the classifier was higher in responder than non-responder samples in the training set. (FIG. 8D) The prediction score of the classifier was higher in responder than non-responder samples in the validation set.

FIG. 9A-D. GO analyses of gene signatures associated with all stages of CRC patients who received FOLFIRI chemotherapy. GO includes three categories: Biological Process, Molecular Function, and Cellular Component. (FIG. 9A) represents the biological process that gene signatures are involved in; (FIG. 9B) Molecular function that the gene signatures are enriched in; (FIG. 9C) the cellular components that the gene signatures are correlated with. (FIG. 9D) represents the KEGG pathways associated with gene signatures identified in metastatic CRC patients who received FOLFOX chemotherapy.

FIG. 10A-D. Construction of LASSO model. (FIG. 10A) Ten-fold cross-validation for tuning parameter selection in the LASSO model. (FIG. 10B) LASSO coefficient profiles of the training set. (FIG. 10C) The prediction score of the classifier was higher in responder than non-responder samples in the training set. (FIG. 10D) The prediction score of the classifier was higher in responder than non-responder samples in the validation set.

FIG. 11A-D. GO analyses of gene signatures associated with CRC patients who received FOLFIRI chemotherapy. GO includes three categories: Biological Process, Molecular Function, and Cellular Component. (FIG. 11A) represents biological process that genes signatures are involved in; (FIG. 11B) Molecular function that the gene signatures are enriched in; (FIG. 11C) the cellular components that the gene signatures are correlated with. (FIG. 11D) represents the KEGG pathways associated with gene signatures identified in metastatic CRC patients who received FOLFOX chemotherapy.

FIG. 12A-D. Construction of LASSO model. (FIG. 12A) Ten-fold cross-validation for tuning parameter selection in the LASSO model. (FIG. 12B) LASSO coefficient profiles of the training set. (FIG. 12C) The prediction score of the classifier was higher in responder than non-responder samples in the training set. (FIG. 12D) The prediction score of the classifier was higher in responder than non-responder samples in the validation set.

FIG. 13A-D. GO analyses of gene signatures associated with metastatic CRC patients who received FOLFIRI chemotherapy. GO includes three categories: Biological Process, Molecular Function, and Cellular Component. (FIG. 13A) represents biological process that genes signatures are involved in; (FIG. 13B) Molecular function that the gene signatures are enriched in; (FIG. 13C) the cellular components that the gene signatures are correlated with, (FIG. 13D) and KEGG pathways.

FIG. 14A-F. Kaplan-Meier survival plots of the three-gene prognostic signature for the gene panel for (FIG. 14A) early-stage CRC patients receiving FOLFOX, (FIG. 14B) metastatic stage CRC patients receiving FOLFOX, (FIG. 14C) early-stage CRC patients receiving FOLFIRI, (FIG. 14D) metastatic stage CRC patients receiving FOLFIRI generated using the GEPIA2 platform. Lines indicate the high- and low-risk patient groups, respectively. Patients were grouped according to median cut-off value for overall survival. In FIG. 14E, GEPIA2 analysis demonstrated that the overall survival (OS) of patients with upregulation of the 11 genes identified in the early-stage CRC patients treated with FOLFIRI are predicted to be more than 80 months longer than the OS of patients treated counter to the prediction (p-value=0.046). In FIG. 14F, the GEPIA2 analysis demonstrated that the overall survival (OS) of patients with upregulation of the 33 genes identified in the metastatic CRC patients treated with FOLFIRI are predicted to be more than 80 months longer than the OS of patients treated counter to the prediction (p-value=0.047).

FIG. 15. Protein-protein interaction networks. The network derived from IMEx interactome database (using NetworkAnalyst web-based visual analytics platform) shows interactions among the gene signatures that are associated to CRC patients who received FOLFOX drug. Genes in red represent the identified genes from the feature selection methods. Protein-protein interaction network shows interactions among the gene signatures that are associated to CRC patients who received FOLFOX drug.

FIG. 16. Protein-protein interaction networks. The network derived from IMEx interactome database (using NetworkAnalyst web-based visual analytics platform) shows interactions among the gene signatures that are associated to early-stage CRC patients who received FOLFOX drug. Genes in red represent the identified genes from the feature selection methods. Protein-protein interaction network shows interactions among the gene signatures that are associated to early-stage CRC patients who received FOLFOX drug.

FIG. 17. Protein-protein interaction networks. The network derived from IMEx interactome database (using NetworkAnalyst web-based visual analytics platform) shows interactions among the gene signatures that are associated to metastatic CRC patients who received FOLFOX drug. Genes in red represent the identified genes from the feature selection methods. Protein-protein interaction network shows interactions among the gene signatures that are associated to metastatic CRC patients who received FOLFOX drug.

FIG. 18. Protein-protein interaction networks. The network derived from IMEx interactome database (using NetworkAnalyst web-based visual analytics platform) shows interactions among the gene signatures that are associated to CRC patients who received FOLFIRI drug. Genes in red represent the identified genes from the feature selection methods. Protein-protein interaction network shows interactions among the gene signatures that are associated to CRC patients who received FOLFIRI drug.

FIG. 19. Protein-protein interaction networks. The network derived from IMEx interactome database (using NetworkAnalyst web-based visual analytics platform) shows interactions among the gene signatures that are associated to CRC patients at early-stage who received FOLFIRI drug. Genes in red represent the identified genes from the feature selection methods. Protein-protein interaction network shows interactions among the gene signatures that are associated to CRC patients at early stage who received FOLFIRI drug.

FIG. 20. Protein-protein interaction networks. The network derived from IMEx interactome database (using NetworkAnalyst web-based visual analytics platform) shows interactions among the gene signatures that are associated to CRC patients at metastatic-stage who received FOLFIRI drug. Genes in red represent the identified genes from the feature selection methods. Protein-protein interaction network shows interactions among the gene signatures that are associated to CRC patients at metastatic stage who received FOLFIRI drug.

FIG. 21. Large panel of significant genes for CRC patients who received FOLFOX treatment.

FIG. 22. Large panel of significant genes for early-stage CRC patients who received FOLFOX treatment.

FIG. 23A-B. Large panel of significant genes (FIG. 23A) for metastatic CRC patients who received FOLFOX treatment (FIG. 23B).

FIG. 24. Large panel of significant genes for CRC patients who received FOLFIRI treatment.

FIG. 25. Large panel of significant genes for early-stage CRC patients who received FOLFIRI treatment.

FIG. 26. Large panel of significant genes for metastatic CRC patients who received FOLFIRI treatment.

### DETAILED DESCRIPTION

Unless defined otherwise, all technical and scientific terms used herein have the same meaning as commonly understood to one of ordinary skill in the art to which this disclosure belongs. Although any methods, devices, and materials similar or equivalent to those described herein can be used in the practice or testing of the methods, devices and materials, the preferred methods, devices, and materials are now described.

### Definitions

The term “microarray” refers to an arrangement of locations on a device. The locations can be arranged in two-dimensional arrays, three-dimensional arrays, or other matrix formats. The number of locations may range from several to at least hundreds of thousands with each location representing a totally independent reaction site. A “nucleic acid array” refers to an array containing nucleic acid probes, such as oligonucleotides or larger portions of genes. The nucleic acid on the array may be single-stranded. As used herein, a nucleic acid or other molecule attached to an array is referred to as a “probe” or “capture probe.”

The term “biological sample,” as used herein, refers to a sample obtained from an organism or from components (e.g., cells) of an organism. The sample may be of any biological tissue or fluid. The sample may be a “clinical sample” which is a sample derived from a patient.

A nucleotide sequence is “complementary” to another nucleotide sequence if each of the bases of the two sequences match, that is, are capable of forming Watson-Crick base pairs. The term “complementary strand” is used herein interchangeably with the term “complement.” The complement of a nucleic acid strand may be the complement of a coding strand or the complement of a non-coding strand.

“Differential gene expression pattern” between, for example, a control cell and a test cell refer to a pattern reflecting the differences in gene expression between the control cell and the test cell. A differential gene expression pattern may also be obtained between a cell at one time point and a cell at another time point, or between a cell derived from a patient treated with a chemotherapeutic drug and a cell derived from the patient prior to drug treatment.

The term “expression profile” refers to a set of values representing mRNA levels of one or more genes in a cell. An expression profile may comprise, for example, values representing expression levels of at least about 2 genes, at least about 5 genes, at least about 10 genes, or at least about 50, 100, 200 or more genes.

The phrase “level of expression” refers to the level of mRNA, as well as pre-mRNA nascent transcript(s), transcript processing intermediates, mature mRNA(s), and degradation products, encoded by a gene in the cell. The phrase “level of expression” also refers to the level of protein or polypeptide in a cell.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., responding to drug treatment) as compared to a biological sample from a subject or group of subjects having a second phenotype (e.g., not responding to a drug treatment). A biomarker may be differentially present at any level, but generally should have an adjusted p-value (also known as FDR) or equal to or less than 0.05 and a Log 2FC change of at least 1 (increase in expression) and below −1 (decrease in expression). Tables 15-20 represent the FDR and Log 2FC levels of expression detected between responders versus non-responders.

As used herein, the terms “comparing” or “comparison” refers to making an assessment of how the level or expression of one or more biomarkers in a sample from a patient compares to levels of expression established for chemotherapy responder and/or non-responder samples.

As used herein, the terms “indicates” or “correlates” (or “indicating” or “correlating,” or “indication” or “correlation,” depending on the context) in reference to a parameter, e.g., the level of expression of a biomarker gene in a sample from a may mean that the patient is likely, or unlikely, to respond to chemotherapy. In specific embodiments, the parameter may comprise the level of expression of one or more biomarkers as disclosed herein.

The terms “measuring” and “determining” are used interchangeably throughout and refer to methods which include obtaining or providing a patient sample and/or detecting the level of biomarker expression in a sample. In certain embodiments, the terms are also used interchangeably with the term “quantitating.”

The present disclosure generally relates to gene expression profiling of tissue samples obtained from colorectal cancer patients who are candidates for chemotherapy treatment. Said samples are obtained are obtained from patients and levels of biomarker expression are compared to established responder versus non-responder levels of biomarker expression. More specifically, the present disclosure provides methods, based on characterization of gene expression, which allow a physician to predict whether a patient is likely to respond well to treatment with a chemotherapeutic reagent. In an embodiment, the chemotherapy reagent is FOLFOX. In yet another embodiment, the chemotherapy reagent is FOLFIRI.

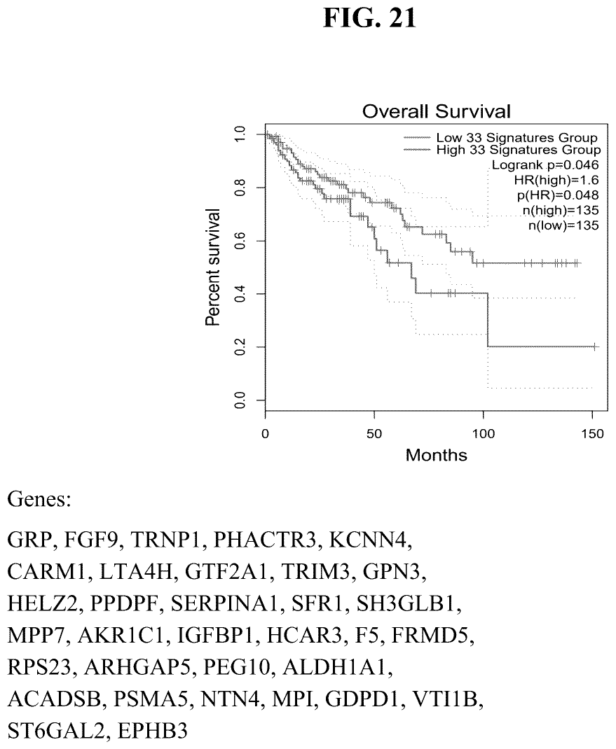

In one embodiment, the disclosure provides a method for predicting the likelihood that patients at all stages of CRC, who are candidates for treatment with FOLFOX, will respond to such treatment, comprising determining the expression level of one or more biomarker transcripts, or their expression product, in a CRC patient sample, wherein the biomarker transcript is the transcript of one or more genes selected from the group consisting of: GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2 and EPHB3. (FIG. 21)

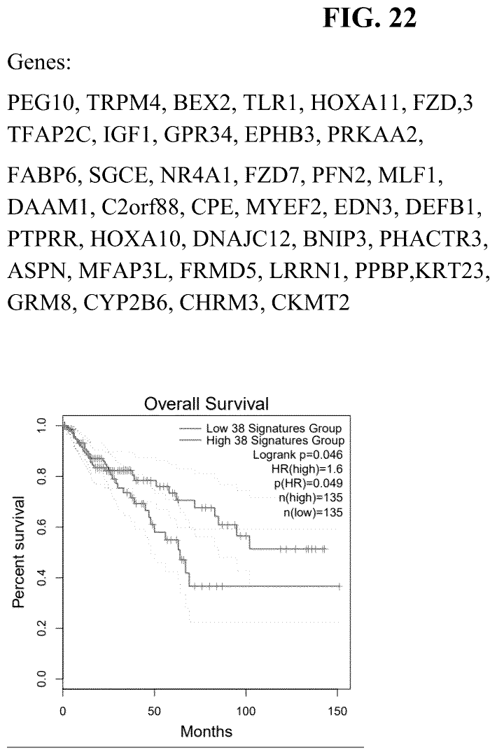

In one embodiment, the disclosure provides a method for predicting the likelihood that patients at early stages of CRC, who are candidates for treatment with FOLFOX, will respond to such treatment, comprising determining the expression level of one or more biomarker RNA transcripts, or their expression product, in a patient sample, wherein the transcript is the transcript of one or more genes selected from the group consisting of: PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2. (FIG. 22)

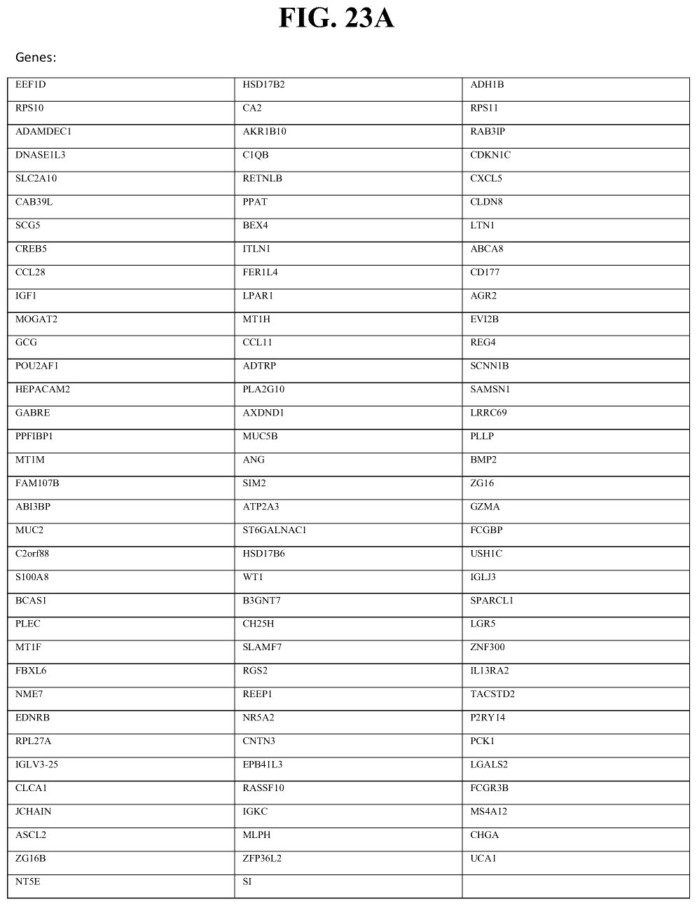

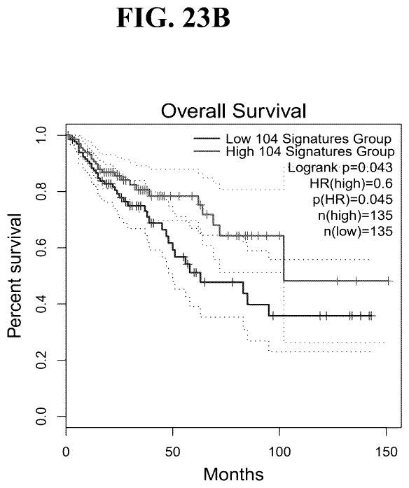

In one embodiment, the disclosure provides a method for predicting the likelihood that patients with metastatic CRC, who are candidates for treatment with FOLFOX, will respond to such treatment, comprising determining the expression level of one or more biomarker RNA transcripts, or their expression product, in a patient sample, wherein the biomarker transcript is the transcript of one or more genes selected from the group consisting of EEF1D, HSD17B2, ADH1B, RPS10, CA2, RPS1,1 ADAMDEC1, AKRIB10, RAB3IP, DNASE1L3, CIQB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL3RA2, NME7, REEP1, TACSTD2, EDNRB, NR5A2, P2RYJ4, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI. (FIG. 23)

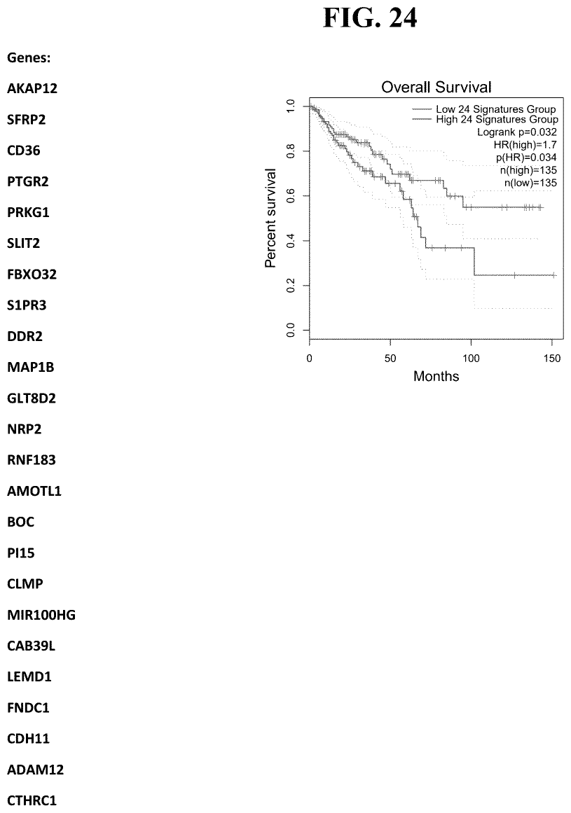

In one embodiment, the disclosure provides a method for predicting the likelihood that patients at all stages of CRC, who are candidates for treatment with FOLFIRI, will respond to such treatment, comprising determining the expression level of one or more biomarker RNA transcripts, or their expression product, in a patient sample, wherein the biomarker transcript is the transcript of one or more genes selected from the group consisting of: AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1. (FIG. 24)

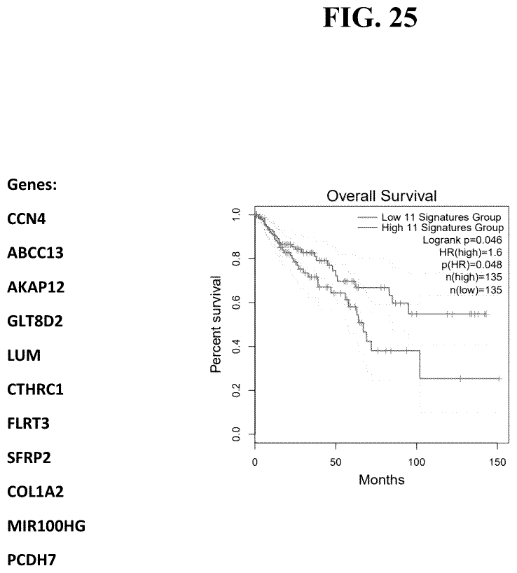

In one embodiment, the disclosure provides a method for predicting the likelihood that patients at early stages of CRC, who are candidates for treatment with FOLFIRI, will respond to such treatment, comprising determining the expression level of one or more biomarker RNA transcripts, or their expression product, in a patient sample, wherein the biomarker transcript is the transcript of one or more genes selected from the group consisting of: including CCN4, ABCC13, AKAP12, GLT8D2, LUM, CTHRC1, FLRT3, SFRP2, COL1A2, MIR100HG, and PCDH7. (FIG. 25)

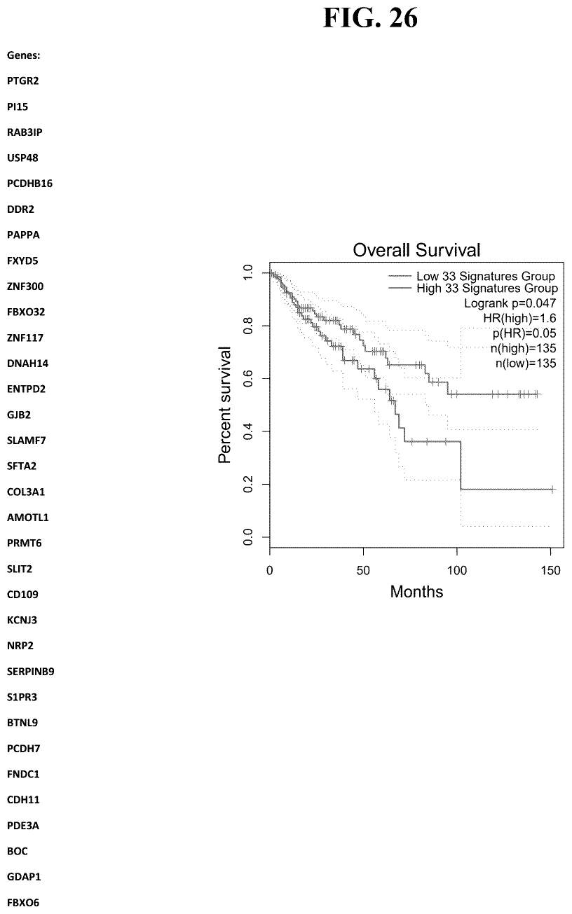

In one embodiment, the disclosure provides a method for predicting the likelihood that patients with metastatic CRC, who are candidates for treatment with FOLFIRI, will respond to such treatment, comprising determining the expression level of one or more biomarker RNA transcripts, or their expression product, in a patient sample, wherein the biomarker transcript is the transcript of one or more genes selected from the group consisting of PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBX06. (FIG. 26)

In one aspect, the disclosure relates to a method for predicting the response of a CRC patient at all stages of CRC to FOLFOX comprising the steps of:
(i) determining in a sample isolated from said patient the expression levels of one or more biomarkers, or their expression product, wherein the biomarker is the transcript, or protein product, of one or more genes selected from the group consisting of: GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKRIC1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2 and EPHB3; and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment.

In one aspect, the disclosure relates to a method for predicting the response of a CRC patient at early stages of CRC to FOLFOX comprising the steps of:
(i) determining in samples isolated from said patient the expression levels of one or more biomarkers, or their expression product, wherein the biomarker is the transcript, or protein product, of one or more genes selected from the group consisting of: PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2 and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment.

In one aspect, the disclosure relates to a method for predicting the response of a CRC patient with metastatic CRC to FOLFOX comprising the steps of:
(i) determining in samples isolated from said patient the expression levels of one or more biomarkers, or their expression product, wherein the biomarker is the transcript, or protein product, of one or more genes selected from the group consisting of: of EEF1D, HSD17B2, ADH1B, RPS10, CA2, RPS1,1 ADAMDEC1, AKRIB10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL3RA2, NME7, REEP1, TACSTD2, EDNRB, NR5A2, P2RYJ4, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment.

In one aspect, the disclosure relates to a method for predicting the response of a CRC patient at all stages of CRC to FOLFIRI comprising the steps of:
(i) determining in samples isolated from said patient the expression levels of one or more biomarkers, or their expression product, wherein the biomarker is the transcript, or protein product, of one or more genes selected from the group consisting of: AKAP12, SFRP2, CD36, PTGR2, PRKG1, SLIT2, FBXO32, SJPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1; and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment.

In one aspect, the disclosure relates to a method for predicting the response of a CRC patient at early stages of CRC to FOLFIRI comprising the steps of:
(i) determining in samples isolated from said patient the expression levels of one or more biomarkers, or their expression product, wherein the biomarker is the transcript, or protein product, of one or more genes selected from the group consisting of: CCN4, ABCC13, AKAP12, GLT8D2, LUM, CTHRC1, FLRT3, SFRP2, COL1A2, MIR100HG, and PCDH7 and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment.

In one aspect, the disclosure relates to a method for predicting the response of a CRC patient with metastatic CRC to FOLFIRI comprising the steps of:
(i) determining in samples isolated from said patient the expression levels of one or more biomarkers, or their expression product, wherein the biomarker is the transcript, or protein product, of one or more genes selected from the group consisting of: PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBXO6 and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment.

In accordance with aspects of the present disclosure, for methods disclosed above the gathered expression data may be compared to one or more threshold values, as disclosed herein, to determine a predicted outcome of whether a patient will respond to FOLFOX or FOLFIRI chemotherapy treatment.

The present disclosure also provides a method of preparing a prognostic profile for a CRC patient using each of the disclosed methods above for predicting the response of a CRC patient to FOLFIRI or FOLFOX. Additionally, each of the disclosed methods above may further comprise the step of creating a report summarizing the data obtained by said gene expression analysis. In yet another embodiment, the disclosed methods above may further comprise the administration of FOLFOX or FOLFIRI where it is determined that the patient is likely to respond to such drug treatment.

The term “reference sample”, “control sample”, as used herein, relates to a sample, which contains reference nucleic acids or proteins to be used as a source of reference nucleic acids or proteins for the methods disclosed herein. In a preferred embodiment, the reference samples are samples derived from chemotherapy responders and/or non-responders. The biomarker nucleic acid or protein levels are then determined in said reference sample and the value obtained is then compared with the levels of the protein or nucleic acid in the patient test sample. This allows the designation of the test sample as “low,” normal” or “high” expression. The collection of samples from which the reference level is derived will preferably be constituted from subjects suffering from the same type of cancer, i.e, CRC and undergoing either FOLFOX or FOLFRI chemotherapy.

In an embodiment, a biomarker expression profile may be developed for a CRC patient, for determining their likelihood of responding to FOLFRI or FOLFOX, based on the expression of the one or more biomarker genes disclosed herein wherein said expression includes both increases and decreases in specific biomarker expression associated with the likelihood of responding to chemotherapy.

In particular embodiments, the methods disclosed herein include collecting a biological sample, such as a primary colorectal tumor sample in which expression of a biomarker genie can be detected. Biological samples may be obtained from a subject by a variety of techniques including, for example, by scraping or swabbing an area, by using a needle to aspirate cells, or by removing a tissue sample (i.e., biopsy). Methods for collecting such biological samples are well known in the art. In some embodiments, a colorectal tumor sample is obtained by, for example, fine needle aspiration biopsy, core needle biopsy, or excisional biopsy. Fixative and staining solutions may be applied to the cells or tissues for preserving the specimen and for facilitating examination. Biological samples, particularly colorectal tumor samples, may be transferred to a glass slide for viewing under magnification. In one embodiment, the biological sample is a formalin-fixed, paraffin-embedded tissue sample, particularly a primary colorectal tumor sample.

The expression levels of the one or more biomarker mRNA transcripts, or their protein products, can be determined by methods known in the art. Methods for detecting expression of the biomarker genes disclosed herein, include methods based on hybridization analysis of polynucleotides, methods based on sequencing of polynucleotides, immunohistochemistry methods, and proteomics-based methods. The methods generally detect expression products (e.g., mRNA or protein) of the biomarker genes. In preferred embodiments, PCR-based methods, such as reverse transcription PCR (RT-PCR), and array-based methods are used.

Many expression detection methods are based on the use of isolated RNA. The starting material is typically total RNA isolated from a biological sample, such as a tumor or tumor cell line, and corresponding normal tissue or cell line, respectively. If the source of RNA is a primary tumor, RNA (e.g., mRNA) can be extracted, for example, from frozen or archived paraffin-embedded and fixed (e.g., formalin-fixed) tissue samples (e.g., pathologist-guided tissue core samples). General methods for RNA extraction are well known in the art and are disclosed in standard textbooks of molecular biology, including Ausubel et al., ed., Current Protocols in Molecular Biology, John Wiley & Sons, New York 1987-1999. The isolated RNA may be further processed for further purification or selection, e.g., selection for mRNA.

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the isolated RNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to a biomarker gene transcript, or any derivative DNA or RNA. Hybridization of the isolated RNA with the probe indicates that the biomarker gene in question is being expressed. In an embodiment, the nucleic acid probes are designed to hybridize to the biomarker gene transcripts disclosed herein.

In one embodiment, the RNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in a gene chip array. A skilled artisan can readily adapt known RNA detection methods for use in detecting the level of expression of the biomarker genes of the present disclosure.

Reagents for detecting the biomarker include one or more reagents for detecting the RNA expression level of the biomarker in the sample, or a reagent for detecting the protein expression level of the biomarker in the sample. Reagents for detecting the RNA expression level of the biomarker in the sample includes reagents used in methods that include, but are not limited to, PCR-based detection method, southern hybridization methods, northern hybridization method, dot hybridization method, fluorescence in situ hybridization method, DNA microarray methods, PCR-ASO probe method, high-throughput sequencing platform methods, and chip methods. In an embodiment, the reagent for detecting the biomarker comprises one or more of a primer for specifically amplifying the biomarker, a probe for specifically recognizing the biomarker, i.e., a nucleic acid probe, and/or a binding agent for specifically binding to a protein encoded by the biomarker.

In an embodiment a method is provided for determining a patient's prognosis for responding to FOLFOX or FOLFIRI chemotherapy comprising the steps of: (i) providing a nucleic acid probe comprising a nucleotide sequence having at least 10, at least 15, at least 25 or at least 40 consecutive nucleotides complementary to the one or more biomarkers disclosed herein, the expression levels of which correlate to a CFC patient's likelihood of responding to FOLFOX or FOLFIRI chemotherapy; (ii) contacting the nucleic acid probe under stringent conditions with the RNA of a patient's tissue sample; and (iii) detecting the amount of hybridization, wherein a difference in the amount of hybridization with the RNA of the patient's test sample as compared to the amount of hybridization of a control test sample is indicative of the patient's prognosis for responding to FOLFOX or FOLFIRI chemotherapy.

To compare expression levels, labeled nucleic acids may be contacted with the test sample under conditions sufficient for binding between the target sample nucleic acid and the probe. In one embodiment, the hybridization conditions may be selected to provide for the desired level of hybridization specificity; that is, conditions sufficient for hybridization to occur between the target sample nucleic acid and probes.

Hybridization may be carried out in conditions permitting essentially specific hybridization. The length and GC content of the nucleic acid will determine the thermal melting point and thus, the hybridization conditions necessary for obtaining specific hybridization of the probe to the target sample nucleic acid. These factors are well known to a person of skill in the art and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed. Elsevier, N.Y., (1993)).

In more particular embodiments, an assay performed on a biological sample obtained from a subject may comprise extracting nucleic acids from the biological sample. The assay can further comprise contacting nucleic acids with one or more primers that specifically bind one or more biomarkers described herein to form a primer:biomarker complex. The assay can further comprise the step of amplifying the primer:biomarker complexes. The amplified complexes can then be detected/quantified to determine a level of expression of the one or more biomarkers. A patient's likelihood of responding to FOLFOX or FOLFIRI chemotherapy can then be identified based on a comparison of the measured levels of one or more biomarkers described herein to one or more reference controls as described herein. The subject can then be treated appropriately, based on the observed levels of gene expression.

In particular aspects, biomarker gene expression is assessed by quantitative RT-PCR. Numerous different PCR or QPCR protocols are known in the art and can be directly applied or adapted for use using the presently described compositions for the detection and/or quantification of the biomarker gene transcripts disclosed herein. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. In certain embodiments, the biomarkers of the present disclosure can be measured by polymerase chain reaction (PCR).

In certain specific embodiments, the present disclosure contemplates quantitation of one or more biomarkers described herein for use in prognosing a CRC patient's response to FOLFOX or FOLFIRI chemotherapy. The one or more biomarkers can be quantitated, and the expression can be compared to reference levels. Overexpression or under expression, depending on the biomarker, relative to the reference is indicative of the likelihood of responding to FOLFOX or FOLFIRI chemotherapy. PCR can include quantitative type PCR, such as quantitative, real-time PCR.

In a specific embodiment, the quantitation steps are carried out using Quantitative PCR (QPCR) (also referred as real-time PCR). One of ordinary skill in the art can design primers that specifically bind and amplify one or more biomarkers described herein using the publicly available sequences thereof. QPCR is preferred under some circumstances because it provides not only a quantitative measurement, but also reduced time and contamination. QPCR gene measurement can be applied to standard formalin-fixed paraffin-embedded clinical tumor blocks, such as those used in archival tissue banks and routine surgical pathology specimens.

In order to normalize the values of mRNA expression among the different samples, it may be desirable to compare the expression levels of the mRNA of interest in the test samples with the expression of a control RNA which is an RNA whose expression levels do not change or change only in limited amounts in tumor cells with respect to non-tumorigenic cells. Such control RNAs may be derived from housekeeping genes and which code for proteins which are constitutively expressed and carry out essential cellular functions. Examples of housekeeping genes for use in the disclosed methods include 0-2-microglobulin, ubiquitin, 18-S ribosomal protein, cyclophilin, GAPDH and β-actin.

In another embodiment, microarrays are used for expression profiling. DNA microarrays provide one method for the simultaneous measurement of the expression levels of large numbers of biomarkers. Each array consists of a reproducible pattern of capture probes attached to a solid support. Labeled RNA or DNA is hybridized to complementary probes on the array and then detected by detection of label. Hybridization intensities for each probe on the array are determined and converted to a quantitative value representing relative gene expression levels. High-density oligonucleotide arrays are particularly useful for determining the gene expression profile for a large number of RNAs in a sample.

By “microarray” is intended an ordered arrangement of hybridizable array elements, such as, for example, polynucleotide probes, on a substrate. The term “probe” refers to any molecule that is capable of selectively binding to a specifically intended target biomarker, for example, a nucleotide transcript or a protein encoded by or corresponding to a biomarker. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules. Arrays can be nucleic acids (or peptides) on beads, gels, polymeric surfaces, fibers (such as fiber optics), glass, or any other appropriate substrate. Arrays can be packaged in such a manner as to allow for diagnostics or other manipulation of an all-inclusive device.

In a specific embodiment of the microarray technique, PCR amplified inserts of cDNA clones are applied to a substrate in a dense array. The biomarker genes, immobilized on the microchip, are suitable for hybridization under stringent conditions. Fluorescently labeled cDNA probes can be generated through incorporation of fluorescent nucleotides by reverse transcription of RNA extracted from the colorectal tumor tissue of interest. Labeled cDNA probes applied to the chip hybridize with specificity to each spot of DNA on the array. After stringent washing to remove non-specifically bound probes, the chip is scanned for detection of label. The quantitation of hybridization of each arrayed element allows for assessment of corresponding mRNA abundance.

In a specific aspect, microarrays are provided comprising one or more probes corresponding to biomarker genes demonstrated to have altered expression following exposure to FOLFOX or FOLFIRI. In an embodiment, the microarray may be a microarray comprising one or more of the genes disclosed herein the expression levels of which correlate to a CFC patient's likelihood of responding to FOLFOX or FOLFIRI chemotherapy. In another aspect, methods are provided for using said microarrays to provide a patient's prognosis for responding to FOLFOX or FOLFIRI chemotherapy. The microarray may comprise, for example, probes corresponding to at least 2, at least 5, at least 10, at least 100 biomarker genes characteristic of the expression levels of which correlate to FOLFOX or FOLFIRI efficacy. The microarray may comprise probes corresponding to each biomarker gene or gene product disclosed herein.

The methods described above result in the production of hybridization patterns of labeled target nucleic acids on the array surface. The resultant hybridization patterns of labeled nucleic acids may be visualized or detected in a variety of ways, with the particular manner of detection selected based on the label of the target nucleic acid. Representative detection means include scintillation counting, autoradiography, fluorescence measurement, colorimetric measurement, light emission measurement, light scattering, and the like.

Any conventional method can be used within the context of the present disclosure to quantify the levels of biomarker protein. For example, biomarkers can be detected and/or measured by immunoassays, mass spectroscopy, western blots and other proteomic detection methods known to one of skill in the art. By way of non-limiting example, the levels of said proteins can be quantified by means of conventional methods, for example, using antibodies with a capacity to specifically bind to biomarker protein (or to fragments thereof containing antigenic determinants) and subsequent quantification of the resulting antibody-antigen complexes.

Such immunoassays require bio-specific capture reagents/binding agent, such as antibodies, to capture the biomarkers. Many antibodies are available commercially. The present disclosure contemplates traditional immunoassays including, for example, sandwich immunoassays including ELISA or fluorescence-based immunoassays, immunoblots, Western Blots (WB), as well as other enzyme immunoassays. Binding of the antigen to the antibody results in changes in absorbance, which is measured.

In specific embodiments, the levels of expression of the biomarkers are determined by contacting the biological sample with antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety. The detection can be performed using a second antibody to bind to the capture antibody complexed with its target biomarker.

The antibodies to be employed in these assays can be, for example, polyclonal sera, hybridoma supernatants or monoclonal antibodies, antibody fragments, Fv, Fab, Fab′ y F(ab′)2, ScFv, diabodies, triabodies, tetrabodies and humanized antibodies. The antibodies can be labeled or not. Examples of labels which can be used include radioactive isotopes, enzymes, fluorophores, chemiluminescent reagents, enzymatic substrates or cofactors, enzymatic inhibitors, particles, colorants, etc. There are a wide variety of well-known assays that can be used, which use non-labeled antibodies (primary antibody) and labeled antibodies (secondary antibodies); among these techniques are included Western-blot or Western transfer, ELISA (enzyme linked immunosorbent assay), RIA (radioimmunoassay), competitive EIA (enzymatic immunoassay), DAS-ELISA (double antibody sandwich ELISA), immunocytochemical and immunohistochemical techniques, techniques based on the use of biochips or protein microarrays.

The present disclosure also provides kits which are suitable for the determination of the expression levels of the biomarker genes disclosed herein. These kits are useful for analyzing a sample from a patient suffering from CRC and to design personalized therapies for said patients based on the results obtained. In a particular embodiment, the reagents of the kit are capable of specifically detecting the levels of the mRNA encoded by a biomarker gene as disclosed above. In another embodiment, the reagents of the kit are capable of specifically detecting the levels of a biomarker protein as disclosed above. The kits may be designed for use with a specific type of CRC patient, e.g., any stage of CRC, early stage of CRC or metastatic CRC. Such kits may be in a microarray format and interface with data analysis operations disclosed below and may be analyzed using a computing system as disclosed below.

In a specific embodiment a kit is designed for predicting the likelihood that a patient at any stage of CRC, who are candidates for treatment with FOLFOX, will respond to such treatment, wherein said kit is designed to determine the expression level of one or more biomarker transcripts, or their expression product, in a CRC patient sample, wherein the biomarker transcript is the transcript of one or more genes selected from the group consisting of: GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VT11B, ST6GAL2 and EPHB3. (FIG. 21)

In one embodiment, a kit is designed for predicting the likelihood that patients at early stages of CRC, who are candidates for treatment with FOLFOX, will respond to such treatment, wherein said kit is designed to determine the expression level of one or more biomarker RNA transcripts, or their expression product, in a CRC patient sample, wherein the biomarker transcript is the transcript of one or more genes selected from the group consisting of: PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2. (FIG. 22)

In one embodiment, a kit is designed for predicting the likelihood that patients with metastatic CRC, who are candidates for treatment with FOLFOX, will respond to such treatment, wherein said kit is designed to determine the expression level of one or more biomarker RNA transcripts, or their expression product, in a CRC patient sample, wherein the biomarker transcript is the transcript of one or more genes selected from the group consisting of EEF1D, HSD17B2, ADH1B, RPS10, CA2, RPS1,1 ADAMDEC1, AKRIB10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL13RA2, NME7, REEP1, TACSTD2, EDNRB, NR5A2, P2RYJ4, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SL (FIG. 23)

In one embodiment, a kit is designed for predicting the likelihood that patients at all stages of CRC, who are candidates for treatment with FOLFIRI, will respond to such treatment, wherein said kit is designed to determine the expression level of one or more biomarker RNA transcripts, or their expression product, in a CRC patient sample, wherein the biomarker transcript is the transcript of one or more genes selected from the group consisting of: AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1. (FIG. 24)

In one embodiment, a kit is designed for predicting the likelihood that patients at early stages of CRC, who are candidates for treatment with FOLFIRI, will respond to such treatment, wherein said kit is designed to determine the expression level of one or more biomarker RNA transcripts, or their expression product, in a CRC patient sample, wherein the biomarker transcript is the transcript of one or more genes selected from the group consisting of: including CCN4, ABCC13, AKAP12, GLT8D2, LUM, CTHRC1, FLRT3, SFRP2, COL1A2, MIR100HG, and PCDH7. (FIG. 25)

In one embodiment, a kit is provided for predicting the likelihood that patients with metastatic CRC, who are candidates for treatment with FOLFIRI, will respond to such treatment, wherein said kit is designed to determine the expression level of one or more biomarker RNA transcripts, or their expression product, in a CRC patient sample, wherein the biomarker transcript is the transcript of one or more genes selected from the group consisting of PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBX06. (FIG. 26).

In certain embodiments, the kit may further include instructions for using the kit, solutions for suspending or fixing cells derived from the sample, detectable tags or labels and solutions for lysing cells. In some instances, the kit may contain solutions and reagents for detecting the RNA or protein products of the biomarker genes.

Following the data gathering operation, the data will typically be reported to a data analysis operation. To facilitate the sample analysis operation, the data obtained by the reader from the device may be analyzed using a computing system. Typically, a computing system will be appropriately programmed for receipt and storage of the data from the device, as well as for analysis and reporting of the data gathered. The computing system may be any system capable of performing computations, including, but not limited to, a desktop, a laptop, a server, a smartphone, a smart watch, a tablet, a wearable device, a cloud system, a standalone system, or other type of computing system, or may be any circuit capable of performing computations, including, but not limited to, an application specific integrated circuit (ASIC), a microprocessor, a microcontroller, a central processing unit (CPU), a graphics processing unit (GPU), a field-programmable gate array, and/or a programmable logic device, among other circuits. In various embodiments, a computing system may include one or more processors and one or more memory storing instructions which, when executed by the one or more processors, implement one or more of the computations and/or procedures disclosed in the present disclosure.

The gathered data may be pre-processed by a computing system in various ways. For example, data which may be an outlier, as compared to previously known data or reference data, may be identified as an outlier and may not be further processed. As another example, various data processing may be employed, such as, without limitation, corrections, transformations, and/or normalizations. Examples of data corrections, transformation, and normalizations are described below herein. Such examples are merely illustrative and do not limit the scope of the present disclosure.

In accordance with aspects of the present disclosure, the gathered data may be used by a computing system to compute a prediction score. Various prediction scores are disclosed below herein. The prediction scores may be compared to one or more threshold values to determine a predicted outcome. For example, if a prediction score has a value greater than a threshold value, the predicted outcome may be that a person would be responsive to chemotherapy, and if the prediction score has a value less than a threshold value, the predicted outcome may be that the person would non-responsive to chemotherapy. These predicted outcomes are merely an example, and other predicted outcomes are contemplated to be within the scope of the present disclosure.

In accordance with aspects of the present disclosure, a computing system may implement a trained machine learning model, and the trained machine learning model may process the gathered data to infer whether the gathered data reflects a predicted outcome. Various machine learning models, and various computations supporting such machine learning models, are disclosed below herein. Persons skilled in the art will understand how to implement and use such machine learning models and computations. In various embodiments, the machine learning model may be a classifier that classifies whether the gathered data is reflective of responsiveness to chemotherapy or is reflective of non-responsiveness to chemotherapy. In various embodiments, the machine learning model may be a regression model that provides an output value reflective of responsiveness and/or non-responsiveness to chemotherapy. The output value may, for example, be compared to one or more threshold values to determine the predicted outcome. The machine learning models disclosed herein are merely examples, and other machine learning models are contemplated to be within the scope of the present disclosure.

In one embodiment, a system may be utilized that comprises a processing function that identifies specific patterns, for example, patterns relating to differential gene expression, for example, between the expression profile of a responder CRC tissue sample and the expression profile of a counterpart non-responder CRC tissue sample. The system may identify patterns of gene expression between more than two samples. Various algorithms are available for analyzing gene expression profile data, for example, the type of comparisons to perform, such as the algorithms disclosed below herein.

Comparison of the expression levels of one or more biomarkers characteristic of FOLFOX or FOLFIRI efficacy with reference expression levels, for example, expression levels in cells of responder CRC patients, non-responder CRC patients or in normal counterpart cells, may be conducted using computing systems. In one embodiment, expression levels may be obtained from two different tissue samples and the two sets of expression levels may be introduced into a computing system for comparison. For example, one set of expression levels is entered into a computing system for comparison with values that are already present in the computing system, or in computer-readable form that is then entered into the computing system.

In one embodiment, the computing system may also contain a database comprising values representing levels of expression of one or more biomarkers characteristic of FOLFOX or FOLFIRI efficacy. The database may contain one or more expression profiles of genes characteristic of small molecule efficacy in different cells.

The present disclosure also provides a machine-readable, processor-readable, or computer-readable medium including program instructions for performing the following steps: (i) comparing a plurality of values corresponding to expression levels of one or more biomarkers characteristic of FOLFOX or FOLFIRI efficacy in a test sample with a database including records comprising reference expression or expression profile data of one or more reference samples and an annotation of the type of sample; and (ii) indicating to which sample the test sample cell is most similar based on similarities of expression profiles. The reference cells may also be cells from subjects responding or not responding FOLFOX or FOLFIRI chemotherapy.

The skilled person will not have problems in selecting a suitable statistical method to evaluate the biomarker marker combinations as disclosed herein and thereby obtain a suitable mathematical algorithm. In this embodiment, data obtained from analysis of biomarker gene expression is evaluated using one or more pattern recognition algorithms.

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE

### 1. Materials and Methods

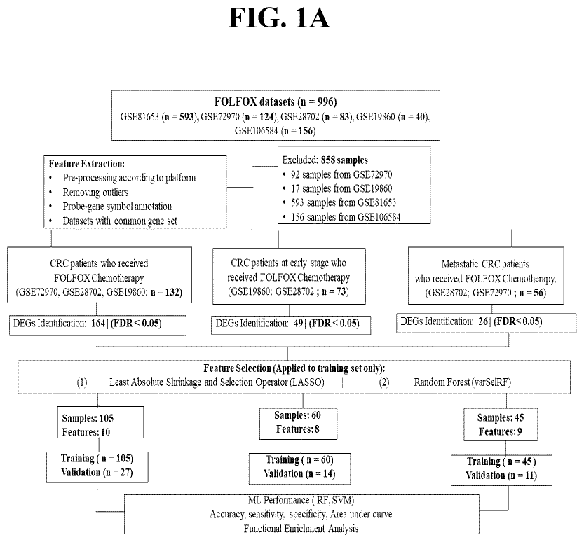

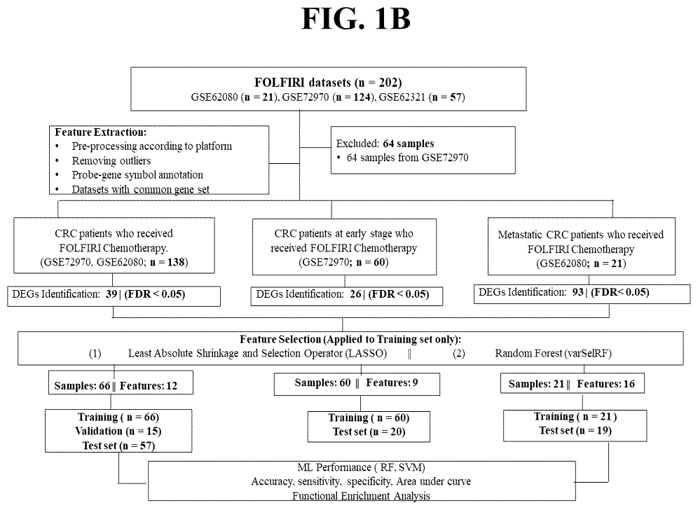

The analysis applied in the study required multiple stages (FIG. 1). First, the secondary gene expression profiling datasets of human colorectal tissues w collected from the NCBI-GEO database. Then the robust multi-array average method in R was used to identify differentially expressed genes (DEGs). LASSO and varSelRF methods were used for feature selection to identify gene signature related to each chemotherapy drug (i.e., FOLFOX or FOLFIRI). Machine learning model performance using random forest and support vector machine algorithms was evaluated. To identify significantly enriched pathways and Gene Ontology (GO) terms, functional enrichment analysis of the gene signatures was performed. Protein-protein interaction networks were reconstructed around these gene signatures. Survival analysis was performed on the gene sets thus identified.

### 1.1 Data

In this study, the raw data (CEL-files) of the colon cancer gene expression datasets was retrieved from the public functional genomics data repository NCBI-GEO database (ncbi.nlm.nih.gov/geo/), using the getGEO function implemented in the R library GEOquery. Affy package in R was used to transform the CEL files of the tumor samples into an expression matrix. “Colon-Cancer”, “Chemotherapy”, “Expression profiling by array”, and “Homo-sapiens” were used as keywords to query all the experimental studies that have probed the gene expression profile within colon tumors of patients who are responders to the drug against those who are not responders. The chemotherapy regimens of interest FOLFOX and FOLFIRI. The Response Evaluation Criteria in Solid Tumors (RECIST) version 1.1 was used to classify response groups. Patients with a complete response (CR) or partial response (PR) were classified to the responder group and patients with stable disease (SD) or progressive disease (PD) were classified to the non-responder group. This approach yielded five different studies, from which the samples of two chemotherapy types (FOLFOX and FOLFIRI) were separated and grouped accordingly.

Table 1 shows the GEO accession numbers of the expression datasets, the platform used for the data set, the numbers of samples (responders, non-responders, total) and the reference of the manuscripts for each dataset that are included in this study after inclusion/exclusion criteria were applied as described in the next section.

TABLE 1
Description of each dataset for two
different Chemotherapy regimens.

| | | Number of samples | | | |
|---|---|---|---|---|---|
| GEO | | Res- | Non- | | Refer- |
| Accession | Platform | ponders | responders | Total | ences |
| GSE19860 | GPL570 - | 15 | 25 | 40 | — |
| GSE72970 | Affymetrix | 63 | 61 | 124 | [22-24] |
| | Human | | | | |
| GSE28702 | Genome U133 | 42 | 41 | 83 | [20] |
| | Plus 2.0 | | | | |
| GSE62080 | Array data | 9 | 12 | 21 | [24, 25] |
| | GPL96 - | | | | |
| GSE62321 | Affymetrix Human | 26 | 31 | 57 | [26] |
| | Genome U133B | | | | |
| | Array | | | | |

### 1.2. Inclusion and Exclusion Criteria

The inclusion criteria in this study were set as follows: (1) patients with colorectal cancer; (2) patients who received FOLFOX or FOLFIRI chemotherapy regimen; (3) microarray expression profiling datasets; (4) sample size of at least 15 for each dataset; (5) available information about the drug response (i.e., responder to the drug vs non-responder to the drug). Exclusion criteria were as follows: (1) datasets contain cell-line or xenograft samples; (2) samples who received preoperative bevacizumab therapy or other immunotherapy; (3) samples with missing information about the drug type; (4) samples with missing information about the drug response; (5) and samples who received a drug combination of FOLFOX and FOLFIRI such as FOLFOXIRI.

### 1.3. Machine Learning Framework

The machine learning framework used to predict the chemotherapy response includes the followings steps: data integration and pre-processing, data splitting using 5-fold cross validation, and feature selection.

### 1.4. Data Integration, Pre-Processing, and Feature Extraction

Once the suitable datasets for this study were identified, the raw microarray expression data for each dataset was obtained from the GEO database. The normalization of the Affymetrix datasets was performed by using Guanine Cytosine Robust Multi-Array Analysis (GCRMA), within the R/Bioconductor packages gcrma (version 2.44.0) [27], for the HG-U133A and HG-U133 Plus 2 platform types The GCRMA algorithm employed in this study conducts several data processing steps, including background correction, log 2 transformation, quantile normalization, and summarization of probe sets into gene-level expression values. This algorithm has demonstrated strong performance in terms of accurately capturing biological variation and enhancing the comparability of data across different platforms. [28, 29]. To ensure consistency across all datasets, certain samples were excluded from the non-normalized data. To accomplish this, the “normalizeBetweenArray” function from the R package limma (version 3.50) [30] was utilized. This function facilitated the proper execution of the aforementioned steps in the data normalization process. Probes with minor sample variance and low median expression levels were removed from RMA data using the nsFilter function, within the R/Bioconductor packages genefilter (version 1.60.0)[31].

After the corresponding pre-processing, each normalized dataset underwent QC implementing the outlier removal strategy. The ArrayQualityMetrics R package [32] was used for quality control and assurance of all microarray experiments. The identification of outliers was carried out using the following statistical measures: i) calculating the mean absolute difference of M-values (log-ratios) for each pair of arrays, ii) determining the Kolmogorov-Smirnov statistic Ka, which measures the difference between an array's signal intensity distribution and the distribution of the pooled data, and iii) calculating the Hoeffding's statistic Da based on the joint distribution of A (average) and M values for each array. This approach has been demonstrated to enhance the effectiveness of meta-analysis and increase the ability to detect differentially expressed genes[33]. During the quality control process, samples that were flagged as outliers in at least two of the three metrics were excluded from their respective datasets. Following this, the raw data that did not contain any outliers underwent a fresh round of normalization using the method described in the previous section. These normalized datasets were then utilized for subsequent analysis.

To establish consistency across platforms, all probes were mapped and associated with gene symbols as the universal identifier. The gene symbols used were official and approved by the HUGO Gene Nomenclature Committee (HGNC) [34]. The utilization of HGNC-approved nomenclature is highly recommended as it undergoes careful curation and has been demonstrated to enhance accuracy in scientific and public communication [35]. In cases where multiple probes were assigned to the same gene symbol, the expression level of that particular gene was determined by calculating the average expression values across all the different probes. Probes that lacked any annotations were not considered in this study. To establish the mapping between probe sets and their corresponding gene symbols, specific annotation packages for each array model were utilized from the Bioconductor repository. The conversion process from probes to gene symbols was accomplished using the R/Bioconductor packages org.Hs.eg.db (version 3.14) [36]. The datasets were annotated using the R/Bioconductor packages hgu133a.db or hgu133plus2.db, depending on the platform.

To standardize the gene expression data, the scale function from the R package stats was employed to apply the Z-score transformation. This classical normalization method allowed for the data to be standardized across various experiments, facilitating the comparison of microarray data irrespective of the initial hybridization intensities [37]. Additionally, the Z-score transformation offers several advantages, including its simplicity, low time and memory complexity, and the absence of assumptions regarding the data distribution. It has been widely employed in previous studies and has consistently shown high performance in various applications. [38].

### 1.5. Data Splitting Using 5-Fold Cross Validation Method

The machine learning model is first trained on a training dataset and subsequently evaluated on a validation dataset to assess its performance. In situations where the dataset is limited, a cross-validation procedure is frequently employed. This involves iteratively dividing the data into training and validation sets to train and assess the model. On the other hand, a test dataset is an entirely separate and independent dataset that has not been utilized in any capacity during the training and validation phases of the model. Using the function “create folds” available in the R package “caret”, samples were randomly split to the training and test set. The training set is split into 5 subsets of approximately equal size.

### 1.6. Feature Selection Using LASSO and varSelRF

In large-scale machine learning applications, the process of feature selection is pivotal in harnessing the advantages of big data while effectively addressing the challenges and costs involved. It plays a significant role in enhancing machine learning applications in several ways. Firstly, it enables faster computation by utilizing a smaller set of features. Secondly, it improves prediction accuracy by eliminating irrelevant features and preventing overfitting. Lastly, it facilitates easier interpretation of the models as it focuses solely on the most important set of features. A wide array of feature selection methods exists for condensing the feature set, broadly categorized into filter methods, wrapper methods, and embedded methods. Filter methods, wrapper methods, and embedded methods serve different purposes in selecting relevant features. In this study, both filter and embedded methods were employed to identify the relevant variables associated with the response to FOLFOX/FOLFIRI drug treatment.

The variable selection using random forest (varSelRF) and Least Absolute Shrinkage and Selection Operator (LASSO) methods were employed to select the genes with the best predictive power. These methods were chosen due to their ability to identify a small subset of genes that exhibit high predictive power. Additionally, they offer the advantage of requiring minimal parameter tuning, as the default parameter values often yield optimal performance.

The varSelRF method, which is based on random forest, utilizes regression trees for classification purposes. The construction of the classification tree involves the use of bootstrap samples, where each branch of the tree consists of a distinct set of candidate variables that are randomly chosen. This approach combines bootstrap aggregation (bagging) and feature selection techniques within the random forest framework, resulting in the generation of trees in varSelRF. To ensure low-bias trees, each tree is constructed independently, followed by the application of bagging and random variable selection techniques to reduce correlation among the trees. The ntree parameter, representing the number of trees, was set to its default value of 2000, while the mtry parameter, determining the number of variables considered at each split, was also set to its default value. [39].

LASSO is a regularization regression method used to fit a generalized linear model. It applies a penalty, specifically the L1-norm, to the regression model, resulting in the reduction of the regression coefficient for variables that contribute minimally to zero. LASSO demonstrates excellent performance in scenarios where the dataset has high dimensionality and a low sample size, and when only a small number of variables possess substantial coefficients. Extensive research has consistently demonstrated LASSO's potential as a promising feature selection model [40, 41]. LASSO regression analysis was performed using the R package glmnet (V4.1)[42].

Using the outcomes obtained, the regression coefficients were utilized to create a scoring system that assigns weights to the selected signature. The formula employed for this purpose is as follows:

$$Prediction Score = {\sum }_{i = 0}^{n} ( {β}_{i} \times {x}_{i} ) \qquad ( 1 )$$

In the given formula, “n” denotes the sample size, while “P” represents the regression coefficient associated with the selected signature. The regression coefficient is obtained through LASSO logistic regression. Additionally, “x” signifies the expression value corresponding to the selected signature.

### 1.7. Machine Learning Algorithms for Classification

The R packages RandomForest and e1071 were used to train two different machine learning algorithms: a random forest and a support vector machine (SVM). To compare the efficacy of the models, the following metrics were measured:

$$Accuracy = \frac{( TP + TN )}{( TN + FN + FP + TP )} \qquad ( 2 )$$

$$Sensitivity = \frac{TP}{( TP + FN )} \qquad ( 3 )$$

$$Specificity = \frac{TN}{( TN + FN )} \qquad ( 4 )$$

The classification model generates predictions for each chemotherapy regimen response, distinguishing between true positive (TP), true negative (TN), false negative (FN), and false positive (FP) outcomes. In this context, responders (R) are considered positive, while non-responders (NR) are regarded as negative. To facilitate comparative analysis, the area under the curve (AUC) metric was employed on the validation and test datasets. Finally, the optimized machine learning model, tailored to predict responses to FOLFOX and FOLFIRI drugs, was applied to the test dataset.

All computational methods and figure generation were implemented using R language programming version 4.0.1. on an Intel Core-i9 CPU with 16 GB of RAM, and 64-bit Windows 10 configuration. The computations could for the machine learning could be run in approximately 1 hour.

### 1.8. Functional Enrichment Analysis

To investigate the association between the predictors of the model and cellular function, a functional enrichment analysis was conducted using the web tool NetworkAnalyst (https://www.networkanalyst.ca/last accessed on 15 Jan. 2023) [43]. NetworkAnalyst web-interface was used to visualize the interactions among the gene products based on the protein-protein interaction (PPI) data in the International Molecular Exchange Consortium (IMEx) database using the default parameters and first-order network. IMEx is a curated database containing non-redundant set of interaction data from a broad taxonomic range of organism [44]. The gene ontology (GO) categories including biological process (BP), molecular function (MF), and cellular component (CC) with false discovery rate (FDR)≤0.05 were identified from the gene ontology database based on the PPI networks derived through IMEX. The pathways that incorporate these gene products (with false discovery rate (FDR)≤0.05) were retrieved from the Kyoto Encyclopedia of Genes and Genomes (KEGG) pathway database [45].

### 1.9. Prognostic Analysis

Moreover, this study leveraged publicly available bioinformatics tools to assess the prognostic value of the identified key hub genes. The Gene Expression Profiling Interactive Analysis (GEPIA2) tool [46], which provides interactive exploration of RNA sequencing data from The Cancer Genome Atlas (TCGA) [47] and the Genotype-Tissue Expression (GTEx) [48] projects, was employed for this purpose. GEPIA2 allowed us to evaluate the prognostic significance of the key hub genes in terms of overall survival (OS) in the TCGA-COAD patient cohort. To determine the difference in survival rates between the high-expression and low-expression groups for each key hub gene, this study employed the log-rank test. Statistical significance was considered when the p-value was less than 0.05, and cut-off criteria such as the median or quartile were used. Survival curves with calculated hazard ratios (HR) and log-rank p-values were plotted. Finally, this study used the GEPIA2 platform to validate the prognostic value of the gene signature generated by the multivariate Cox regression analysis and to visualize the corresponding survival curves.

### 2. Results

The goal of this study was to use baseline tumor gene expression to predict patients' response to drugs. An overview of the approach is shown in FIG. 1. A series of meta-analyses were performed to develop a machine learning model and identify biomarkers to predict the following: 1) FOLFOX responders vs non-responders in all stages of CRC, 2) FOLFOX responders vs non-responders at early stages of CRC, 3) FOLFOX responders vs non-responders among patients with metastatic CRC, 4) responders vs non-responders in patients in all stages of CRC who received FOLFIRI chemotherapy, 5) responders vs non-responders in patients at early stages of CRC who received FOLFIRI chemotherapy, 6) responders vs non-responders in patients with metastatic CRC who received FOLFIRI chemotherapy, and 7) machine learning model application to predict effectiveness of alternate chemotherapy regimen. All datasets in this study are identified by unique GEO accession numbers which are provided in the material and methods section. Each GEO submission file includes a brief overview of the experimental paradigm as well as a link to the published report, if available.

### 2.1. FOLFOX Responders Vs Non-Responders at all Stages of CRC

In the initial analysis of colorectal cancer patients, significant genes were identified that could distinguish between individuals who responded to FOLFOX chemotherapy and those who did not respond. Notably, the stage of the disease did not play a significant role in this analysis. The purpose of this analysis was to compare the genes discovered in this study with those previously identified in other studies, thus expanding understanding of the molecular factors involved in FOLFOX chemotherapy response. To obtain a comprehensive dataset for the analysis, three datasets (GSE28702, GSE19860, and GSE72970) were merged. These datasets were generated using the Affymetrix microarray GPL570 platform. Specifically, they included colorectal cancer (CRC) patients who received FOLFOX chemotherapy, resulting in a total of 67 non-responders and 65 responders. Before conducting the analysis, samples from the GSE72970 dataset that received the FOLFIRI drug were excluded. Subsequently, a cross-validation method was employed to divide the combined dataset into a training set and a validation set. The training set consisted of 105 samples in total, with 53 non-responders and 52 responders. The validation set comprised 27 samples, including 14 non-responders and 13 responders.

After integrated bioinformatics analysis, total of 164 differentially expressed genes (DEGs) between pre-chemotherapy tissue samples of non-responders and responders of CRC patients treated with FOLFOX were identified including 142 upregulated genes and 22 down-regulated genes.

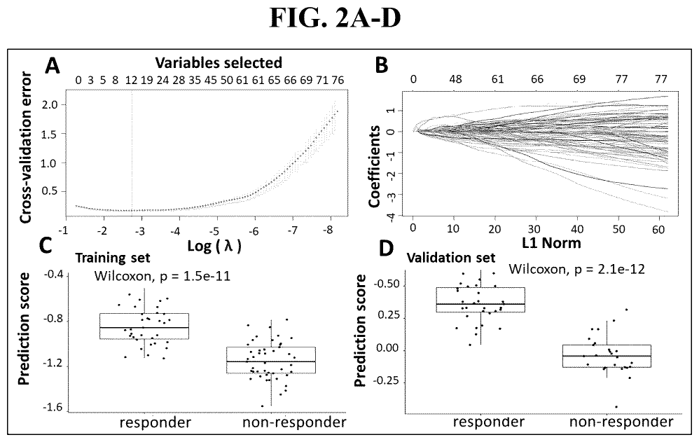

The expression values of 164 specifically chosen genes from the training set were extracted and subjected to analysis using the LASSO regression model. To select an optimal λ, ten-fold cross-validations were performed to calculate the cross-validation error. The value of λ corresponding to the minimum cross-validation error was selected as the optimal λ (denoted as λ min). A dotted vertical line was drawn at the value chosen by 10-fold cross-validation. The optimal λ min value, 0.0651 results in 12 non-zero coefficients (FIG. 2A-B). Thus, the 12-gene signatures were selected from 164 DEGs including CFAP92, LTA4H, SH3GLB1, CARM1, PPDPF, GPN3, TRIM3, HELZ2, APZ51, LOC100652999, GTF2A1, and LLPH. Further, this study compared the prediction scores of the 12-gene signature classifier between responders and non-responders' samples, and the results showed that it could distinguish the two groups of samples well, both in the training and test sets (FIG. 2C-D). The prediction scores were higher in responders' samples than no responders' samples in both sets. These results demonstrated that samples that differed in response to FOLFOX-based chemotherapy were more easily distinguished by the 12-gene signature classifier. The prediction score for the signature classifier is calculated using the following formula:

$$Prediction Score = CFAP 92 \times ( 0.0707023790552402 ) + LTA 4 H \times ( - 0.0351545549822743 ) + SH 3 GLB 1 \times ( - 0.0408850596289773 ) + CARM 1 \times 0.383628162287505 + PPDPF \times 0.080928167572473 + GPN 3 \times 0.0586206766352588 + TRIM 3 \times 0.161462309025774 + HELZ 2 \times 0.01687976195595 + LRRC 3 \times 0.438014842725689 + LOC 100652999 \times 0.14063710120844 + GTF 2 A 1 \times 0.0776912702849236 + AP 5 Z 1 \times ( - 0.022687801677234 ) \qquad ( 5 )$$

Furthermore, the feature selection technique varSelRF was employed to analyze the differentially expressed genes (DEGs). This approach resulted in gene signatures composed of the following genes: CFAP92, LTA4H, SH3GLB1, CARM1, PPDPF, GPN3, TRIM3, HELZ2, GTF2A1, and AP5Z1. Although both the LASSO and varSelRF models identified distinct sets of markers, there were a few genes that were commonly identified by both methods. The presence of these commonly identified genes, which are closely associated with colorectal cancer, emphasizes the biological significance of the model.

Through a progressive testing process, these methods evaluated the genes and gradually narrowed down the selection to identify the optimal gene set. The optimal gene set was determined based on its ability to achieve the best prediction performance. Ten genes were identified as relevant genes from both methods, including Cilia And Flagella Associated Protein 92 (CFAP92), Leukotriene A4 Hydrolase (LTA4H), SH3 Domain Containing GRB2 Like, Endophilin B1 (SH3GLB1), Adaptor Related Protein Complex 5 Subunit Zeta 1 (AP5Z1), Coactivator Associated Arginine Methyltransferase 1 (CARM1), Tripartite Motif Containing 3 (TRIM3), Pancreatic Progenitor Cell Differentiation And Proliferation Factor (PPDPF), GPN-Loop GTPase 3 (GPN3), General Transcription Factor HA Subunit 1 (GTF2A1), and Helicase With Zinc Finger 2 (HELZ2).

The performance evaluation of the models was conducted on both the training and validation sets using metrics such as accuracy, sensitivity, specificity, and AUC. The results, presented in Table 2, demonstrated that the top-performing machine learning algorithm was random forest. However, there was no significant difference between the random forest and SVM algorithms. These machine learning algorithms were applied to the validation set to predict the response to FOLFOX chemotherapy, and the corresponding prediction outcomes were recorded in Table 2. The number of patient records for the training and validation modes was indicated as n=105 and n=27, respectively.

In terms of accuracy, random forest achieved a score of 1 (95% CI: (0.95, 1)), indicating perfect accuracy for the given data, while SVM attained a score of 0.96 (95% CI: (0.75, 1)). In addition, when considering sensitivity, specificity, and AUC (Area Under the Curve), the random forest method exhibited superior performance compared to the SVM method. All these evaluation metrics achieved a value of 1, indicating excellent accuracy in correctly distinguishing between responders and non-responders. However, the SVM algorithm demonstrated comparable performance with a sensitivity of 0.92, specificity of 1, and an AUC of 0.96, indicating its strong predictive capabilities.

TABLE 2
Comparison of various classification methods on training and validation
sets of CRC patients at all stages who received FOLFOX treatment
using the combination of LASSO and varSelRF method.

FOLFOX (LASSO & VarSelRF)

| | | Random | Support Vector |
|---|---|---|---|
| Model | | forest (RF) | Machine (SVM) |
| Training | Accuracy | 1 | 0.92 |
| (n = 105) | 95% CI | (0.95, 1) | (0.77, 0.95) |
| | Sensitivity | 1 | 0.92 |
| | Specificity | 1 | 0.84 |
| Validation | Accuracy | 1 | 0.96 |
| (n = 27) | 95% CI | (0.95, 1) | (0.75, 1) |
| | Sensitivity | 1 | 0.92 |
| | Specificity | 1 | 1 |
| | AUC | 1 | 0.96 |

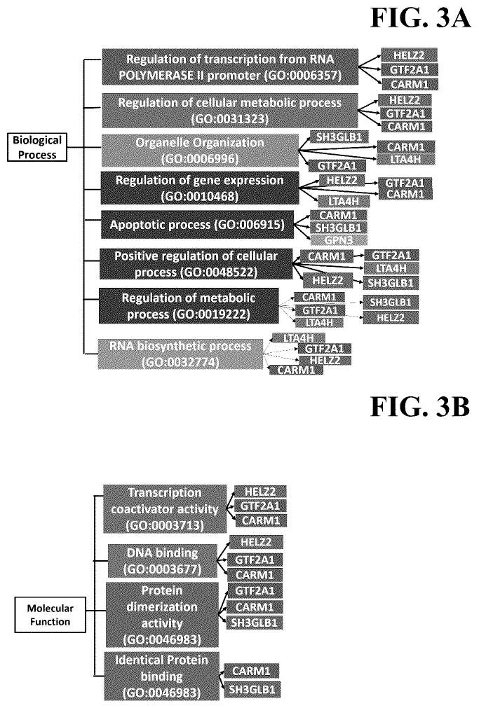

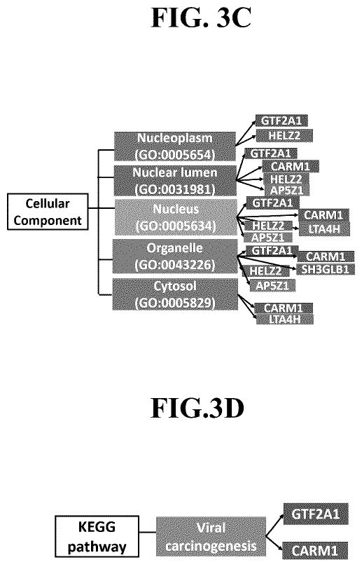

To gain a deeper understanding of the potential pathophysiological significance of the gene signatures involved in colorectal cancer (CRC) development, functional annotation and enrichment analysis were conducted. This analysis focused on the target genes associated with these gene signatures and utilized the gene ontology biological process (GO-BP), molecular functions (GO-MF), cellular component terms (GO-CC), and canonical pathways from the KEGG database (FIG. 3). The GO categories and KEGG pathway analyses were established using the Protein-Protein Interaction (PPI) network derived from IMEx—NetworkAnalyst tool. The analysis yielded a significant number of enriched functional categories. Some of these categories are broad in nature and involve a substantial number of target genes. Examples of such categories include regulation of transcription from RNA polymerase II promoter (GO: 0006357); regulation of cellular metabolic process (GO:0031323); organelle organization (GO:0006996); regulation of gene expression (GO:0010468); apoptotic process (GO:006915); and positive regulation of cellular process (GO:0048522) for GO-BP terms, transcription co-activator activity (GO: 0003713); DNA binding (GO:0003677); protein dimerization (GO:0046983) for GO-MF, nucleoplasm (GO: 0005654); nuclear lumen (GO:0031981); nucleus (GO:0005634); organelle (GO:0043226); cytosol (GO:0005829) for GO-CC, and viral carcinogenesis (hsa05203) for KEGG pathways (FIG. 3).

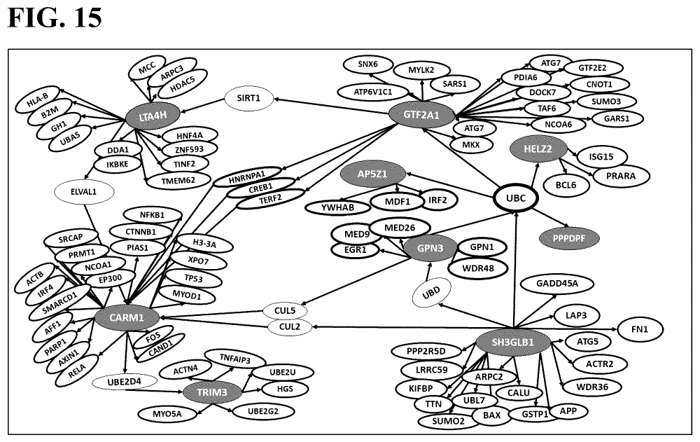

The gene signatures were used to construct the IMEx-generated protein-protein interaction (PPI) network. This network provides information about the interactions (both direct and indirect) among the gene-encoded proteins, as depicted in FIG. 15. The IMEx consortium gathers experimental evidence of interactions directly from original research articles and carefully curates a non-redundant dataset comprising physical and molecular interaction data. The size of the nodes represents their degree centrality values. Nodes in the network that exhibit a larger degree centrality are considered key nodes or hubs of biological significance. FIG. 15 shows a PPI network consisting of 208 nodes (genes connected to other genes) and 216 edges (connections between nodes), with 5 out of 10 genes identified as hub genes due to their numerous connections with other genes. Notably, CARM1, LTA4H, GTF2A1, TRIM3, and SH3GLB1 exhibit the highest number of interactions with other genes. According to the PPI network predicted using IMEx, the gene-encoded proteins corresponding to the signature genes do not have any known direct functional impact on each other. CARM1 is linked to LTA4H, TRIM3, SH3GLB1, and GPN3 through ELVAL1, UBE2D4, CUL2, and CUL5, respectively. Additionally, CARM1 is connected to GTF2A1 via TERF2, CREB1, and HNRNPA1. Furthermore, LTA4H interacts with GTF2A1 through SIRT1, while GPN3 is connected to SH3GLB1 via the UBD gene-encoded protein. TRIM3 and CARM1 genes are connected through UBE2D4. GPN3, SH3GLB1, AP5Z1, HELZ2, PPPDPF, and GTF2A1 directly interact with UBC.

Furthermore, the analysis was expanded to include additional genes that play a significant role in predicting the response to drug treatment. Some of these genes were not initially identified through the feature selection methods (LASSO and varSelRF) during the initial phase. Out of the 164 genes that exhibited differential expression, 33 genes, namely GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2, and EPHB3, displayed significant differences in their ability to predict the response to FOLFOX treatment between individuals who responded positively and those who did not.

The model's performance was evaluated using both the training and validation datasets. As shown in Table 3, the random forest algorithm emerged as the most effective machine learning technique. Subsequently, these machine learning algorithms were applied to the validation set to make predictions regarding the response to FOLFOX treatment, and the prediction outcomes were presented in Table 3. The RF and SVM algorithms achieved an accuracy of 0.89. Additionally, the random forest algorithm ranked first in sensitivity (0.88), specificity (0.87), and had an AUC of 0.89. The SVM algorithm showed comparable results to the random forest algorithm with a sensitivity of 0.87, specificity of 0.88, and an AUC of 0.89.

TABLE 3
Comparison of different classification methods on training and
validation sets of CRC patients at all stages who received FOLFOX
treatment using the large panel of significant genes.

FOLFOX (LASSO & VarSelRF)

| | | Random | Support Vector |
|---|---|---|---|
| Model | | forest (RF) | Machine (SVM) |
| Training | Accuracy | 0.90 | 0.91 |
| (n = 105) | 95% CI | (0.85, 0.98) | (0.77, 0.95) |
| | Sensitivity | 0.91 | 0.92 |
| | Specificity | 0.89 | 0.84 |
| Validation | Accuracy | 0.89 | 0.89 |
| (n = 27) | 95% CI | (0.75, 0.93) | (0.78, 0.94) |
| | Sensitivity | 0.88 | 0.87 |
| | Specificity | 0.87 | 0.88 |
| | AUC | 0.89 | 0.89 |

### 2.2. FOLFOX Responders Vs Non-Responders at Early Stages of CRC

To conduct a more detailed analysis, a subgroup analysis was performed due to the inclusion of datasets containing both primary and metastatic lesions. In this analysis, the focus was solely on the samples from primary tumor tissues. The aim of this analysis was to identify genes that could distinguish between responders and non-responders in the early stages of cancer, thus providing insights into the predictive factors specific to early-stage colorectal cancer.

The datasets GSE28702 and GSE19860, obtained from GPL570, consisted of 74 primary colorectal cancer (CRC) samples from patients who received first-line FOLFOX-based treatment. For this analysis, metastasis samples from the GSE28702 dataset were excluded. Additionally, samples from the GSE19860 dataset that received a combination of FOLFOX and Bevacizumab were excluded as well. Among the datasets, 60 samples (27 responders and 33 non-responders) were used as the training set, while the remaining 14 samples (7 responders and 7 non-responders) were used as the validation set. Following an integrated bioinformatics analysis, a total of 49 DEGs were identified between pre-chemotherapy tissue samples of non-responders and responders in CRC patients treated with FOLFOX. Among these genes, 38 were upregulated, while 11 were downregulated.

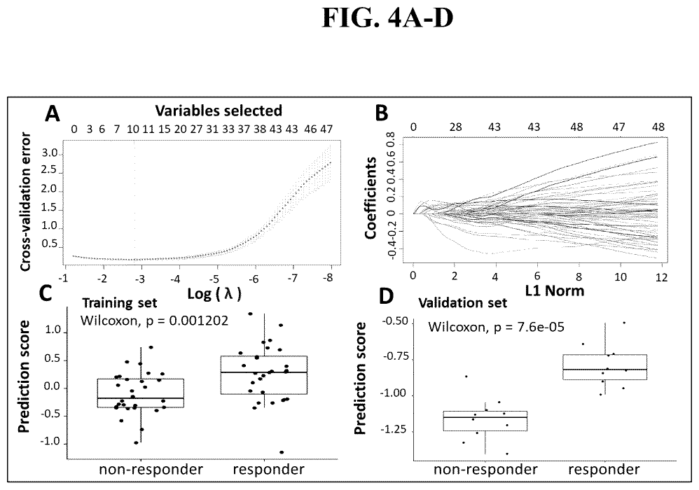

To build the model to predict sensitivity to FOLFOX-based chemotherapy in colorectal cancer, the patients in the training dataset were grouped into responders or non-responders groups based on the patient response status, and the expression values of the selected 49 genes from training set were extracted and analyzed by LASSO regression model analysis. To select an optimal λ, ten-fold cross-validations were performed to calculate the cross-validation error. A dotted vertical line was drawn at the value chosen by 10-fold cross-validation. The optimal λ min value, 0.0605 results in 10 non-zero coefficients (FIGS. 4A, B). Thus, the 10-gene signatures were selected from 49 DEGs including NR4A1, CYP2B6, TRPM4, GRM8, HOXA11, HOXA10, CHRM3, LINC02086, FABP6, and BEX2. Furthermore, this study compared the prediction scores of the 10-gene signature classifier between responders and non-responders' samples, and the results showed that it could distinguish the two groups of samples well, both in the training and test sets (FIG. 4C, D). The prediction scores were higher in responders' samples than no responders' samples in both sets. These results demonstrated that samples that differed in response to FOLFOX-based chemotherapy were more easily distinguished by the 10-gene signature classifier. The prediction score for the signature classifier is calculated using the following formula:

$$Prediction Score = CHRM 3 \times 0.0252272039743314 + NR 4 A 1 \times 0.147656847485106 + CYP 2 B 6 \times 0.0609620384129753 + FABP 6 \times ( - 0.0329157407927049 ) + HOXA 11 \times 0.0501485865470492 + GRM 8 \times 0.0545018052768938 + TRPM 4 \times 0.0802540599090828 + BEX 2 \times ( - 0.0342169746495821 ) + HOXA 10 \times 0.115227059404438 + LINC 02086 \times 0.0685852089429017 \qquad ( 2 )$$

Additionally, the feature selection technique, varSelRF, was utilized on the differentially expressed genes (DEGs). The gene signatures obtained from varSelRF encompassed the following genes: HOXA10, TRPM4, NR4A1, LINC02086, HOXA11, CYP2B6, PEG10, GRM8, EPHB3, and CHRM3. Although both models identified a diverse set of markers, only a few gene candidates were commonly identified by both methods. Notably, most of these commonly identified genes are closely linked to colorectal cancer, highlighting the biological significance of the models.

After conducting feature selection methods, the gene signatures were progressively evaluated, and the gene set that exhibited the best prediction performance was identified as the optimal gene set. Eight genes, including NR4A1 (Nuclear Receptor subfamily 4 group A member 1), CYP2B6 (Cytochrome P450 subfamily B member 6), TRPM4 (Transient Receptor Potential cation channel subfamily M member 4), GRM8 (Glutamate Metabotropic Receptor 8), HOXA11 (Homeobox A11), HOXA10 (Homeobox A10), CHRM3 (Cholinergic Receptor Muscarinic 3), and LINC02086 (Long Intergenic Non-Protein Coding RNA 2086), were identified as relevant genes by both methods.

The evaluation of model performance was performed in training and validation sets. As shown in Table 4, the top machine learning algorithm was random forest. These machine learning algorithms were applied to the validation set to predict FOLFOX response, and the prediction results were displayed in Table 4. Random Forest and SVM had an accuracy of 0.93 (95% CI: 0.74, 0.94) and 0.91 (95% CI: 0.83, 0.95) respectively. In addition, random forest ranked first with a sensitivity of 1, specificity of 0.87, and AUC of 0.92. The SVM algorithm was comparable to the random forest algorithm with a sensitivity of 0.90, specificity of 0.83, and AUC of 0.91.

TABLE 4
Comparison of different classification methods on training and validation
sets of CRC patients at early-stage who received FOLFOX treatment
using the combination of LASSO and varSelRF method.

mFOLFOX (LASSO & VarSelRF)

| | | Random | Support Vector |
|---|---|---|---|
| Model | | Forest (RF) | Machine (SVM) |
| Training | Accuracy | 1 | 0.96 |
| (n = 60) | 95% CI | (0.94, 1) | (0.84, 0.99) |
| | Sensitivity | 1 | 1 |
| | Specificity | 1 | 0.86 |
| Validation | Accuracy | 0.93 | 0.91 |
| (n = 14) | 95% CI | (0.74, 0.94) | (0.8303, 0.95) |
| | Sensitivity | 1 | 0.9 |
| | Specificity | 0.87 | 0.83 |
| | AUC | 0.92 | 0.91 |

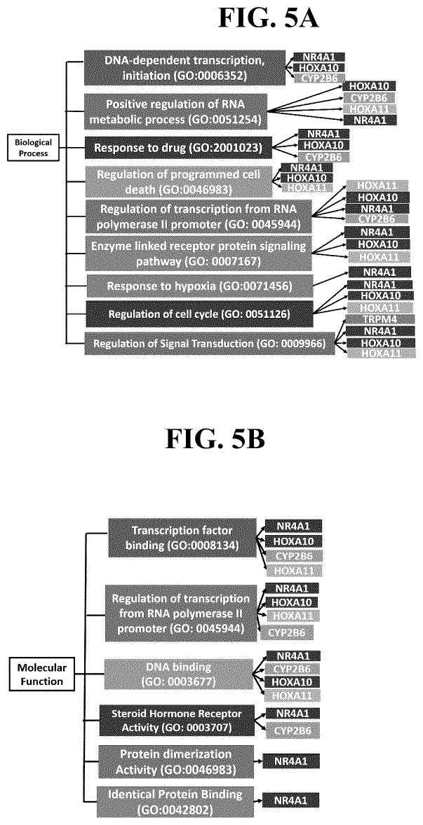

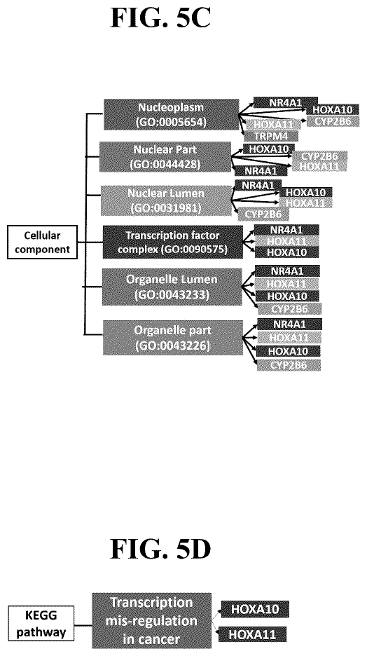

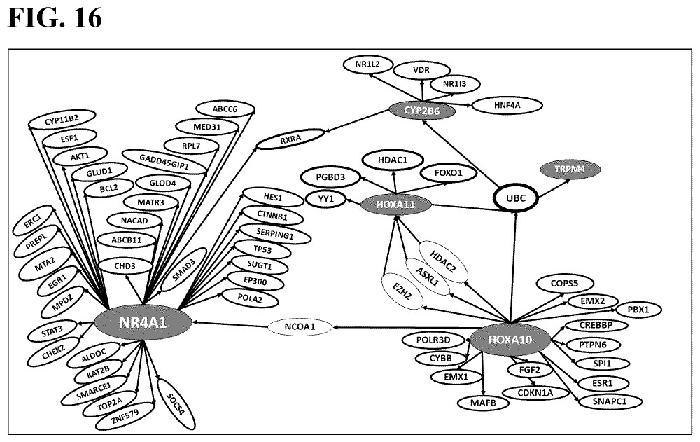

FIG. 5 illustrates the representation of Biological Process (BP), Molecular Function (MF), and Cellular Component (CC), along with the pathways that incorporate these proteins. The BP, MF, and CC terms were obtained from the Gene Ontology (GO) database, while the pathways were identified from the KEGG database. The GO and KEGG pathway analyses were established using the Protein-Protein Interaction (PPI) network derived from IMEx. The significant terms from the GO enrichment analysis highlighted that the gene signatures were associated with various processes in the BP category, including the DNA-dependent transcription, initiation (GO:0006352), positive regulation of RNA metabolic process (GO:0051254), regulation of programmed cell death (GO:0043067), response to drug (GO:2001023), and others. In the MF category, the gene signatures were enriched in transcription factor binding (GO:0008134), Regulation of transcription from RNA polymerase II promoter (GO:0045944), DNA binding (GO:0003677), steroid hormone receptor activity (GO:0003707), protein dimerization activity (GO:0046983), and identical protein binding (GO:0042802). Regarding the CC category, the gene signatures were correlated with specific locations such as nucleoplasm (GO:0005654), nucleus part (GO:0044428), nuclear lumen (GO:0031981), transcription factor complex (GO:0090575), organelle lumen (GO:0043233), and organelle part (GO:0043226). In the KEGG pathway enrichment analysis, the gene signatures were found to be involved in pathways (hsa05200), transcriptional mis-regulation in cancer (hsa05202), apoptosis (hsa04210), MAPK signaling pathway (map04010), and PI3K-AKT signaling pathway (hsa04151) (FIG. 5). The IMEx-generated protein-protein interaction (PPI) network was constructed using the gene signatures. FIG. 16 illustrates a protein-protein interaction (PPI) network, which comprises 138 nodes representing genes connected to one another, along with 141 edges denoting the connections between these nodes. Within this network, 4 out of 10 genes have been identified as hub genes due to their significant number of connections with other genes. Specifically, NR4A1, HOXA10, HOXA11, and CYP2B6 exhibit the highest number of interactions with other genes.

According to the PPI network generated through IMEx predictions, the proteins encoded by these signature genes do not appear to have any known direct functional influence on each other. However, NR4A1 is linked to HOXA10 through NCOA1 and to CYP2B6, HOXA11, and TRPM4 via UBC. Additionally, HOXA10 is connected to HOXA11 through HDAC2, ASXL1, and EZH2. Moreover, NR4A1 interacts with CYP2B6 through RXRA.

Furthermore, this study expanded the analysis to include additional genes that are important in predicting drug response. These genes were not initially identified through the feature selection methods (LASSO and varSelRF) during the first phase. Out of the 49 genes identified as differentially expressed, the analysis found that 38 genes including PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2 exhibited a notable distinction in their ability to predict the response to FOLFOX treatment between individuals who responded positively and those who did not.

The model's performance was assessed using both the training and validation datasets. The predicted outcomes of the models were presented in Table 5. The random forest algorithm achieved an accuracy of 0.90 (95% CI: 0.83, 0.95), while the Support Vector Machine (SVM) algorithm exhibited an accuracy of 0.80 (95% CI: 0.73, 0.85). In addition, random forest ranked first with a sensitivity of 0.94, specificity of 0.78, and AUC of 0.90. The SVM algorithm was comparable to the random forest algorithm with a sensitivity of 0.94, specificity of 0.66, and AUC of 0.80.

TABLE 5
Comparison of different classification methods on training
and validation sets of CRC patients at early-stage who
received FOLFOX treatment using the larger panel of DEGs.

mFOLFOX (LASSO & VarSelRF)

| | | Random | Support Vector |
|---|---|---|---|
| Model | | Forest (RF) | Machine (SVM) |
| Training | Accuracy | 0.92 | 0.91 |
| (n = 60) | 95% CI | (0.90, 0.99) | (0.75, 0.99) |
| | Sensitivity | 1 | 1 |
| | Specificity | 0.97 | 0.9 |
| Validation | Accuracy | 0.90 | 0.80 |
| (n = 14) | 95% CI | (0.83, 0.95) | (0.73, 0.85) |
| | Sensitivity | 0.94 | 0.94 |
| | Specificity | 0.78 | 0.66 |
| | AUC | 0.90 | 0.80 |

### 2.3. FOLFOX Responders Vs Non-Responders Among Patients with Metastatic CRC

This analysis focused on selecting genes separating responders from non-responders in metastatic CRC patients. The GSE72970A dataset, which represents metastatic samples only, was used in this step along with the metastatic samples from the GSE28702 dataset. These two datasets, which were generated by the GPL570 platform, were combined to yield a total of 27 non-responders and 28 responders of metastatic CRC patients with mFOLFOX chemotherapy. Among the data sets, 45 samples (22 responders and 23 non-responders) were used as the training set, while the remaining 11 samples (6 responders and 5 non-responders) were used as the validation set. A gene with an FDR≤0.05 and |log 2FC|≥1 was identified as differentially expressed gene (DEG). To address the issue of false positives resulting from the limited sample size and imbalanced gene expression levels in the training dataset, a bootstrap t-test was employed. In this analysis, genes were identified as differentially expressed if they exhibited a false discovery rate (FDR) of ≤0.05 and an absolute log 2 fold change (|log 2FC|) of ≥1. Following datasets preprocessing, 134 differential expressed genes (DEGs) between pre-chemotherapy tissue samples of non-responders and responders of CRC patients treated with FOLFOX were identified including 101 upregulated genes and 33 down-regulated genes.

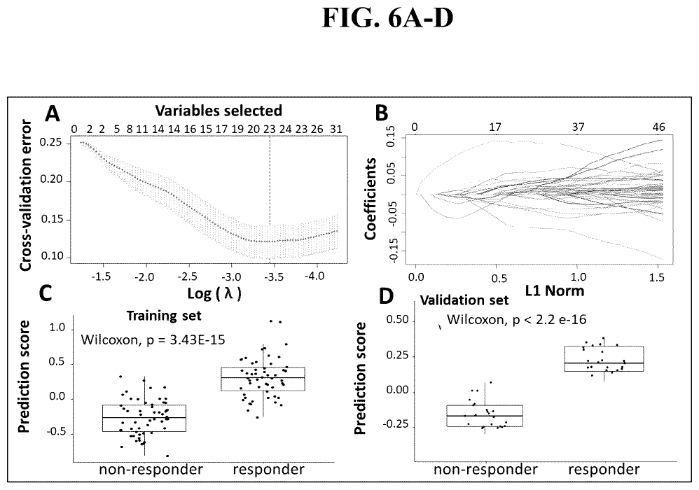

To develop a predictive model for determining the sensitivity of colorectal cancer patients to FOLFOX-based chemotherapy, the metastatic patients in the training dataset were categorized into two groups: responders and non-responders, based on their treatment response status. The expression values of the selected 134 genes from the training set were then extracted and subjected to analysis using the LASSO regression model. To select an optimal λ, ten-fold cross-validations were performed to calculate the cross-validation error. A dotted vertical line was drawn at the value chosen by 10-fold cross-validation. The optimal λ min value, 0.0349 results in 23 non-zero coefficients (FIG. 6A-B). Thus, the 23-gene signatures selected from 134 DEGs included TACSTD2, IQGAP2, REEP1, KR1B10, PPAT, IGF1, CLCA1, CDKN1C, PPFIBP1, EEF1D, BEX4, PLEC, ZFTA, USH1C, FER1L3, ABI3BP, GGTA1, CREB5, LINC02067, LRRC69, RAB3IP, LINC02086, and HSD17B6. Further, this study compared the prediction scores of the 23-gene signature classifier between responders and non-responders' samples, and the results showed that it could distinguish the two groups of samples well, both in the training and test sets (FIG. 6C-D). The prediction scores were higher in responders' samples than no responders' samples in both sets. These results demonstrated that samples that differed in response to FOLFOX-based chemotherapy were more easily distinguished by the 23-gene signature classifier. The prediction score for the signature classifier is calculated using the following formula:

$$Prediction Score = TACSTD 2 \times 0.00270945684650097 + IQGAP 2 \times ( - 0.00847808418322799 ) + REEP 1 \times 0.00605656057956512 + AKR 1 B 10 \times ( - 0.0203366801255001 ) + PPAT \times 0.0201089896480733 + IGF 1 \times ( - 0.0284545651292782 ) + CDKN 1 C \times 0.00741111664384363 + PPFIBP 1 \times ( - 0.0317847003508003 ) + EEF 1 D \times 0.141850458165839 + BEX 4 \times ( - 0.00176740773798336 ) + PLEC \times 0.0222352477700212 + ZFTA \times 0.0102617253057372 + USH 1 C \times 0.0545082812981942 + FER 1 L 4 \times 0.0359442555529826 + ABI 3 BP \times ( - 0.0755005720787851 ) + GGTA 1 \times ( - 0.00944239443492612 ) + CREB 5 \times 0.0351766533783486 + LINC 02067 \times 0.00907100821483412 + LRRC 69 \times 0.0238555594546346 + RAB 3 IP \times 0.000169633620952202 + HSD 17 B 6 \times ( - 0.00214863965806719 ) \qquad ( 7 )$$

Additionally, the feature selection technique, varSelRF, was utilized on the differentially expressed genes (DEGs). The gene signatures obtained from varSelRF encompassed the following genes: TACSTD2, IQGAP2, REEP1, AKRIB10, PPAT, IGF1, CDKN1C, PPFIBP1, EEF1D, BEX4, PLEC, ZFTA, USH1C, FER1L4, ABI3BP, GGTA1, CREB5, LINCO2067, and LRRC69. Although both models identified a diverse set of markers, only a few gene candidates were commonly identified by both methods. Most of these commonly identified genes are closely linked to colorectal cancer, highlighting the biological significance of the models.

After conducting feature selection methods, the gene signatures were progressively evaluated, and the gene set that exhibited the best prediction performance was identified as the optimal gene set. Nine genes, including IQGAP2 (IQ Motif Containing GTPase Activating Protein 2), IGF1 (Insulin Like Growth Factor 1), EEF1D (Eukaryotic Translation Elongation Factor 1 Delta), ZFTA (Zinc Finger Translocation Associated), FER1L4 (Fer-1 Like Family Member 4), ABI3BP (ABI Family Member 3 Binding Protein), GGTA1 (Glycoprotein alpha-Galactosyltransferase 1), LINC02067 (Long Intergenic Non-Protein Coding RNA 2067), and LRRC69 (Leucine Rich Repeat Containing 69), were identified as relevant genes by both methods. The evaluation of model performance was assessed in training and validation sets.

The performance evaluation of the models was conducted on both the training and validation sets. As depicted in Table 6, the top-performing machine learning algorithm was random forest. The predictions of these machine learning algorithms were also applied to the validation set to forecast the response to FOLFOX chemotherapy, and the corresponding prediction outcomes were displayed in Table 6. Random forest achieved an accuracy of 1 (95% CI: 0.72, 1), indicating a high level of accuracy. On the other hand, SVM achieved an accuracy of 0.90 (95% CI: 0.59, 1), suggesting a slightly lower accuracy compared to random forest.

In terms of sensitivity, specificity, and AUC, random forest outperformed SVM. Random forest achieved a sensitivity, specificity, and AUC of 1, implying excellent performance in correctly identifying responders and non-responders. SVM, although slightly less sensitive with a value of 0.86, demonstrated similar performance with a specificity of 1 and an AUC of 0.93. These results indicate that both random forest and SVM possess strong predictive capabilities, with random forest ranking higher in most metrics.

TABLE 6
Comparison of different classification methods
on training and validation sets after features
selection using LASSO and VarSelRF method.

FOLFOX (LASSO & VarSelRF)

| | | Random | Support Vector |
|---|---|---|---|
| Model | | forest (RF) | Machine (SVM) |
| Training | Accuracy | 1 | 0.86 |
| (n = 45) | 95% CI | (0.92, 1) | (0.73, 0.95) |
| | Sensitivity | 1 | 0.77 |
| | Specificity | 1 | 0.92 |
| Validation | Accuracy | 1 | 0.90 |
| (n = 11) | 95% CI | (0.72, 1) | (0.59, 1) |
| | Sensitivity | 1 | 0.86 |
| | Specificity | 1 | 1 |
| | AUC | 1 | 0.93 |

FIG. 7 displays the depiction of Biological Process (BP), Molecular Function (MF), and Cellular Component (CC), along with the pathways in which these gene encoded proteins are involved. The GO and KEGG pathway analyses were conducted based on the Protein-Protein Interaction (PPI) network generated from IMEx. The GO enrichment analysis identified significant terms indicating that the gene signatures were linked to diverse biological processes in the BP category, encompassing the DNA-dependent transcription, initiation (GO:0006352), positive regulation of RNA metabolic process (GO:0051254), regulation of programmed cell death (GO:0043067), response to drug (GO:2001023), and others. In the MF category, the gene signatures were enriched in transcription factor binding (GO:0008134), Regulation of transcription from RNA polymerase II promoter (GO:0045944), DNA binding (GO:0003677), steroid hormone receptor activity (GO:0003707), protein dimerization activity (GO:0046983), and identical protein binding (GO:0042802). Regarding the CC category, the gene signatures were correlated with specific locations such as nucleoplasm (GO:0005654), nucleus part (GO:0044428), nuclear lumen (GO:0031981), transcription factor complex (GO:0090575), organelle lumen (GO:0043233), and organelle part (GO:0043226). In the KEGG pathway enrichment analysis, the gene signatures were found to be involved in pathways (hsa05200), transcriptional mis-regulation in cancer (hsa05202), apoptosis (hsa04210), MAPK signaling pathway (map04010), and PI3K-AKT signaling pathway (hsa04151) (FIG. 7).

The IMEx-generated protein-protein interaction (PPI) network was constructed using the gene signatures. FIG. 17 illustrates a protein-protein interaction (PPI) network, which comprises 120 nodes representing genes connected to one another, along with 121 edges denoting the connections between these nodes. Within this network, 3 out of 10 genes have been identified as hub genes due to their significant number of connections with other genes. Specifically, IQGAP2, ABI3BP, and EEF1D exhibit the highest number of interactions with other genes.

According to the PPI network generated through IMEx predictions, the proteins encoded by these signature genes do not appear to have any known direct functional influence on each other. However, EEF1D is linked to ABI3BP through GRB2 and to IQGAP2, and LRRC69 via UBC. Additionally, EEF1D is connected to IQGAP2 through RELA. Moreover, IQGAP2 interacts with ABI3BP through MYC.

Furthermore, this study expanded the analysis to incorporate additional genes that play a crucial role in predicting drug response. These genes were not initially identified through the feature selection methods (LASSO and varSelRF) in the initial phase. Among the 134 genes that showed differential expression, the analysis found that 104 genes displayed significant variations in their predictive ability for determining the response to FOLFOX treatment between individuals who responded positively and those who did not. The list of genes displayed in the FIGS. 21-26.

The performance evaluation of the model was conducted on both the training and validation datasets. As indicated in Table 5, the random forest algorithm emerged as the most effective machine learning technique. Subsequently, these machine learning algorithms were utilized on the validation set to make predictions regarding the response to FOLFOX treatment, and the outcomes of these predictions were presented in Table 5. Random Forest and Support Vector Machine (SVM) had an accuracy of 0.95 (95% CI: 0.75, 0.99) and 0.85 (95% CI: 0.60, 0.91) respectively. Furthermore, the random forest method achieved the highest sensitivity score of 1, along with a specificity of 0.9 and an AUC value of 0.95. The SVM algorithm yielded a lower performance compared to the random forest algorithm, exhibiting a sensitivity of 0.8, specificity of 0.7, and an AUC value of 0.85.

TABLE 7
Comparison of different classification methods on
training and validation sets using the large panel.

FOLFOX (LASSO & VarSelRF)

| | | Random | Support Vector |
|---|---|---|---|
| Model | | Forest (RF) | Machine (SVM) |
| Training | Accuracy | 0.97 | 0.89 |
| (n = 45) | 95% CI | (0.94, 0.99) | (0.82, 0.0.94) |
| | Sensitivity | 0.98 | 0.92 |
| | Specificity | 0.96 | 0.66 |
| Validation | Accuracy | 0.95 | 0.85 |
| (n = 11) | 95% CI | (0.75, 0.99) | (0.60, 0.91) |
| | Sensitivity | 1 | 0.8 |
| | Specificity | 0.9 | 0.7 |
| | AUC | 0.95 | 0.85 |

### 3.4. Responders Vs Non-Responders Samples Who Received FOLFIRI Chemotherapy at all Stages of CRC

In the fourth analysis of colorectal cancer patients, significant genes were identified that could differentiate between responders and non-responders to FOLFIRI treatment in the metastatic stages of cancer. The training set and validation set used in this analysis comprised a total of 66 and 15 CRC patients, respectively. These patients were selected from the combined dataset (GSE62080 and GSE72970) obtained from the GPL570 platform, and they had received first-line FOLFIRI-based treatment. The combined dataset included samples from 45 non-responders and 36 responders with metastatic CRC. For the independent test data, a dataset called GSE62321 derived from the Affymetrix Human Genome U133B Array (GPL97) platform was used. This dataset included a total of 57 patients, consisting of 31 non-responders and 26 responders. Overall, these datasets were employed to investigate and identify genes that could effectively differentiate between responders and non-responders to FOLFIRI treatment specifically in the context of metastatic colorectal cancer.

Following datasets preprocessing, 40 differential expressed genes (DEGs) between pre-chemotherapy tissue samples of non-responders and responders of CRC patients treated with FOLFIRI were identified including 9 upregulated genes and 31 down-regulated genes.

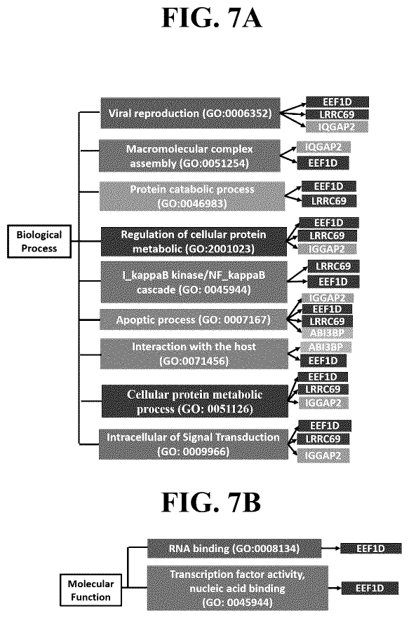

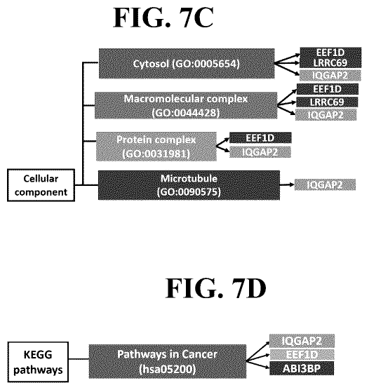

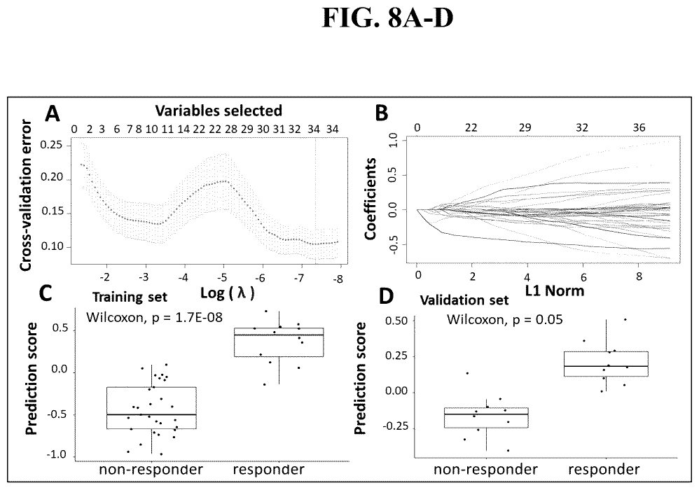

To develop a predictive model for determining the sensitivity of colorectal cancer patients to FOLFIRI-based chemotherapy, the patients in the training dataset were categorized into two groups: responders and non-responders, based on their treatment response status. The expression values of the selected 40 genes from the training set were then extracted and subjected to analysis using the LASSO regression model. To select an optimal λ, ten-fold cross-validations were performed to calculate the cross-validation error. A dotted vertical line was drawn at the value chosen by 10-fold cross-validation. The optimal λ min value, 0.0210 results in 34 non-zero coefficients (FIG. 8A-B). Thus, the 34-gene signatures selected from 40 DEGs included OGN, NRP2, SFRP2, ABI3BP, MND1, TMPRSS3, FBXO32, AMOTL1, RNA45SN5, CAB39L, BOC, MAP1B, CLMP, FNDC1, GLT8D2, WDR72, PAX8.AS1, AKAP12, CACNA2D1, PRKG1, PCDH7, CD36, COL1A2, LINC01614, LEMD1, PI15, PTGR2, COL3A1, RNF183, MIX23, CDH11, C3orf80, and SERPINB9. Further, this study compared the prediction scores of the 34-gene signature classifier between responders and non-responders' samples, and the results showed that it could distinguish the two groups of samples well, both in the training and test sets (FIG. 8C-D). The prediction scores were higher in responders' samples than non-responders samples in both sets. These results demonstrated that samples that differed in response to FOLFIRI-based chemotherapy were more easily distinguished by the 34-gene signature classifier. The prediction score for the signature classifier is calculated using the following formula:

$$Prediction Score = OGN \times ( - 0.026747485 ) + NRP 2 \times ( - 0.479830846 ) + SFRP 2 \times ( - 0.008852721 ) + ABI 3 BP \times ( - 0.251862481 ) + MND 1 \times ( - 0.189794013 ) + SLIT 2 \times ( - 0.158823987 ) + FBXO 32 \times 0.378204031 + RNA 45 SN 5 \times ( - 0.054747404 ) + CAB 39 L \times 0.249095948 + BOC \times ( - 0.077415537 ) + MAP 1 B \times 0.301174128 + CLMP \times 0.052730754 + FNDC 1 \times ( - 0.023013935 ) + GLT 8 D 2 \times ( - 0.21784962 ) + AMOTL 1 \times 0.018417206 + PAX 8. AS 1 \times ( - 0.04543003 ) + AKAP 12 \times 0.876734016 + CACNA 2 D 1 \times 0.12242411 + PRKG 1 \times 0.018402322 + PCDH 7 \times ( - 0.318842155 ) + CD 36 \times ( - 0.104574686 ) + LINC 01641 \times 0.039000746 + LEMD 1 \times ( - 0.228052347 ) + PI 15 \times 0.005342257 + PTGR 2 \times ( - 0.548660753 ) + COL 3 A 1 \times 0.588575905 + RNF 183 \times 0.035670237 + DDR 2 \times 0.02476893 + CDH 11 \times 0.232601528 + C 3 orf 80 \times 0.285287459 + CTHRC 1 \times ( - 0.092173245 ) \qquad ( 8 )$$

Moreover, the feature selection technique, varSelRF, was utilized on the differentially expressed genes (DEGs). The gene signatures obtained from varSelRF encompassed the following genes: TACSTD2, AMOTL1, CTHRC1, FNDC1, DDR2, SLIT2, CDH11, SFRP2, AKAP12, PTGR2, CD36, COL3A1, USH1C, FER1L4, ABI3BP, GGTA1, CREB5, LINC02067, and LRRC69. Although both models identified a diverse set of markers, only a few gene candidates were commonly identified by both methods.

After applying feature selection techniques, the gene signatures underwent a step-by-step evaluation process, leading to the identification of the gene set with the highest predictive performance as the ideal gene set. 12 genes, including Angiomotin Like 1 (AMOTL1), Collagen Triple Helix Repeat Containing 1 (CTHRC1), Fibronectin Type III Domain Containing 1 (FNDC1), Collagen Type I Alpha 2 Chain (COL1A2), Discoidin Domain Receptor Tyrosine Kinase 2 (DDR2), Slit homolog 2 (SLIT2), Cadherin 11 (CDH11), Collagen Type III Alpha 1 Chain (COL3A1), A-Kinase Anchoring Protein 12 (AKAP12), Secreted Frizzled Related Protein 2 (SFRP2), Prostaglandin Reductase 2 (PTGR2), and cluster of differentiation 36 (CD36), were identified as relevant genes by both methods. The evaluation of model performance was assessed in training and validation sets.

The performance evaluation of the models was conducted on the training, validation, and independent test sets. As presented in Table 8, the top-performing machine learning algorithm was random forest. These machine learning algorithms were utilized to predict the response to FOLFIRI chemotherapy in both the validation and independent test sets, and the corresponding prediction outcomes were documented in Table 8.

In the independent test set, both random forest and SVM achieved an accuracy of 0.96 (95% CI: 0.87, 0.99), indicating high levels of accuracy in predicting the response to FOLFIRI treatment. Random forest outperformed SVM in terms of sensitivity, specificity, and AUC. It achieved a sensitivity of 0.89, specificity of 0.92, and an AUC of 0.94, showcasing its strong predictive capabilities. SVM, on the other hand, demonstrated similar performance with a sensitivity of 0.89, specificity of 0.93, and an AUC of 0.93. These results indicate that both random forest and SVM are effective in predicting the response to FOLFIRI chemotherapy, with random forest performing slightly better in terms of sensitivity, specificity, and AUC.

TABLE 8
Comparison of different classification methods
on training and validation sets after features
selection using LASSO and VarSelRF method.

mFOLFIRI (LASSO & VarSelRF)

| | | Random | Support Vector |
|---|---|---|---|
| Model | | Forest (RF) | Machine (SVM) |
| Training | Accuracy | 1 | 0.96 |
| (n = 66) | 95% CI | (0.94, 1) | (0.84, 0.99) |
| | Sensitivity | 1 | 1 |
| | Specificity | 1 | 0.86 |
| Validation | Accuracy | 0.93 | 0.93 |
| (n = 15) | 95% CI | (0.74, 0.94) | (0.83, 0.95) |
| | Sensitivity | 1 | 0.95 |
| | Specificity | 0.87 | 0.86 |
| | AUC | 0.93 | 0.93 |
| Independent | Accuracy | 0.96 | 0.96 |
| Test | 95% CI | (0.87, 0.99) | (0.87, 0.99) |
| (n = 57) | Sensitivity | 0.89 | 0.89 |
| | Specificity | 0.92 | 0.93 |
| | AUC | 0.94 | 0.93 |

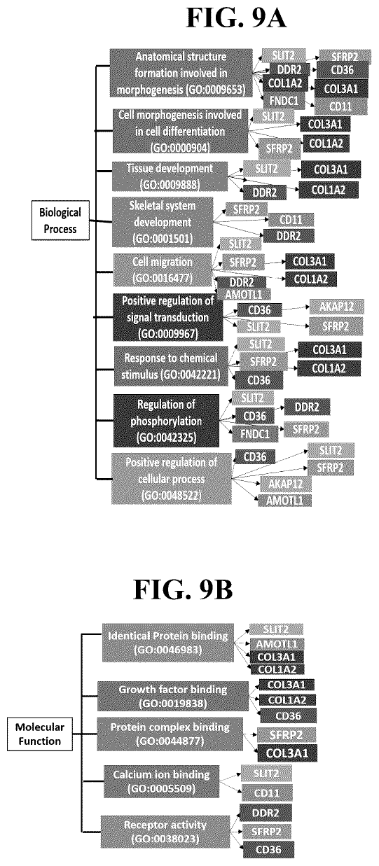

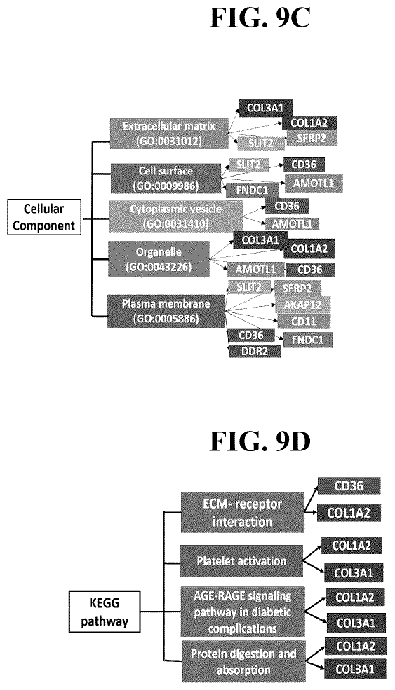

BP, MF, and CC, along with pathways that incorporate these proteins are shown in FIG. 8. The top significant terms from the GO enrichment analysis showed that in the BP category, the gene signatures were involved in anatomical structure formation involved in morphogenesis (GO:0009653), cell morphogenesis involved cell differentiation (GO:0000904), tissue development (GO:0009888), skeletal system development (GO: 0001501), cell migration (GO:0016477), and others. For the MF category, the gene signatures were enriched in identical protein binding (GO:0046983), growth factor binding (GO:0019838), protein complex binding (GO:0044877), calcium ion binding (GO:0005509), and receptor activity (GO:0038023). For the CC category, the gene signatures were correlated with extracellular matrix (GO:0031012), cell surface (GO:0009986), cytoplasmic vesicle (GO:0031410), organelle (GO:0043226), and plasma membrane (GO:0005886). For KEGG pathway enrichment analysis, among the gene signatures, CD36 and COL1A2 were involved in ECM-receptor interaction pathway. COL1A2 and COL3A2 were involved in platelet activation (hsa04512), AGE-RAGE signaling pathway in diabetic complications (hsa04933), and protein digestion and absorption pathway (hsa04974) (FIG. 9).

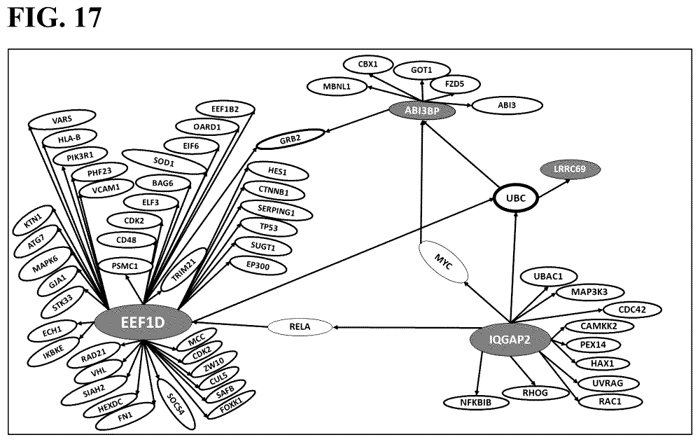

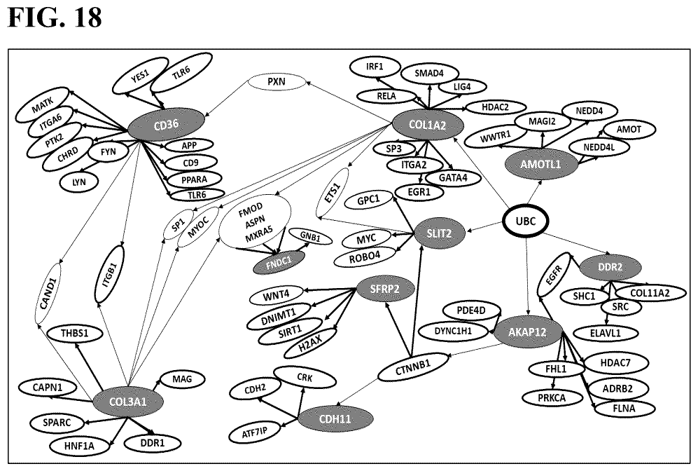

The protein-protein interaction (PPI) networks generated through IMEx indicate (direct and indirect) interactions among these gene encoded proteins (FIG. 18). As shown in FIG. 18, the PPI network comprises 71 nodes and 72 edges with 6 out of 10 genes being hub genes. For instance, SLIT2, AKAP12, AMOTL1, DDR2, COL3A1 and COL1A2 have the highest number of hub genes. Based on the PPI network predicted using IMEx, the signature proteins have no known direct functional effect on each other. COL1A2 connects to COL3A1 via SP1 and MYOC gene encoded protein. COL1A2 also connects to SLIT2 via ETS1. In addition, AMOTL1, COL1A2, DDR2, AKAP12, and SLIT2 interact directly with UBC. On the other hand, AKAP12 connects to CDH11 via CTNNB1.

Furthermore, the analysis was expanded to include additional genes that are important in predicting drug response. These genes were not initially identified through the feature selection methods (LASSO and varSelRF) during the first phase. Out of the 40 genes identified as differentially expressed, 24 genes including AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1 exhibited a notable distinction in their ability to predict the response to FOLFIRI treatment between individuals who responded positively and those who did not. The performance evaluation of the model was conducted on both the training and validation datasets (Table 9). Random Forest and Support Vector Machine (SVM) had an accuracy of 0.85 (95% CI: 0.74, 0.96) and 0.81 (95% CI: 0.67, 0.86) respectively in the validation set. Furthermore, the random forest method achieved a high sensitivity score of 0.95, along with a specificity of 0.87 and an AUC value of 0.85. The SVM algorithm yielded a relatively lower performance compared to the random forest algorithm, exhibiting a sensitivity of 0.84, specificity of 0.76, and an AUC value of 0.81. In addition, in the test set, random forest and SVM had an accuracy of 0.82 (95% CI: 0.57, 0.89) and 0.81 (95% CI: 0.57, 0.88) respectively. The random forest method achieved a sensitivity score of 0.79, along with a specificity of 0.72 and an AUC value of 0.82. The SVM algorithm yielded a relatively lower performance compared to the random forest algorithm, exhibiting a sensitivity of 0.79, specificity of 0.78, and an AUC value of 0.81.

TABLE 9
Comparison of different classification methods on
training and validation sets after features selection
using large panel of significant genes.

FOLFIRI (LASSO & VarSelRF)

| | | Random | Support Vector |
|---|---|---|---|
| Model | | Forest (RF) | Machine (SVM) |
| Training | Accuracy | 0.90 | 0.83 |
| (n = 66) | 95% CI | (0.74, 0.96) | (0.64, 0.93) |
| | Sensitivity | 0.87 | 1 |
| | Specificity | 0.79 | 0.9 |
| Validation | Accuracy | 0.85 | 0.81 |
| (n = 15) | 95% CI | (0.71, 0.88) | (0.67, 0.86) |
| | Sensitivity | 0.95 | 0.84 |
| | Specificity | 0.87 | 0.76 |
| | AUC | 0.85 | 0.81 |
| Independent | Accuracy | 0.82 | 0.81 |
| Test | 95% CI | (0.57, 0.89) | (0.57, 0.88) |
| (n = 57) | Sensitivity | 0.79 | 0.79 |
| | Specificity | 0.72 | 0.78 |
| | AUC | 0.82 | 0.81 |

### 2.5. Responders Vs Non-Responders Samples Who Received FOLFIRI Responders Vs Non-Responders at Early Stages of CRC

The fifth analysis of colorectal cancer patients identified significant genes separating FOLFIRI responders from non-responders for early stages of cancer. The training set consisted of 60 patients who received first-line FOLFIRI-based treatment from the dataset GSE72970 derived from the platform GPL570. This dataset included 60 samples for a total of 33 non-responders and 27 responders of primary CRC samples. The independent test data included 20 patients (11 non-responders and 9 responders) from the dataset GSE62321 derived from the platform Affymetrix Human Genome U133B Array (GPL97). Following an integrated bioinformatics analysis, a total of 26 DEGs were identified between pre-chemotherapy tissue samples of non-responders and responders in CRC patients treated with FOLFIRI. Among these genes, 3 were upregulated, while 23 were downregulated.

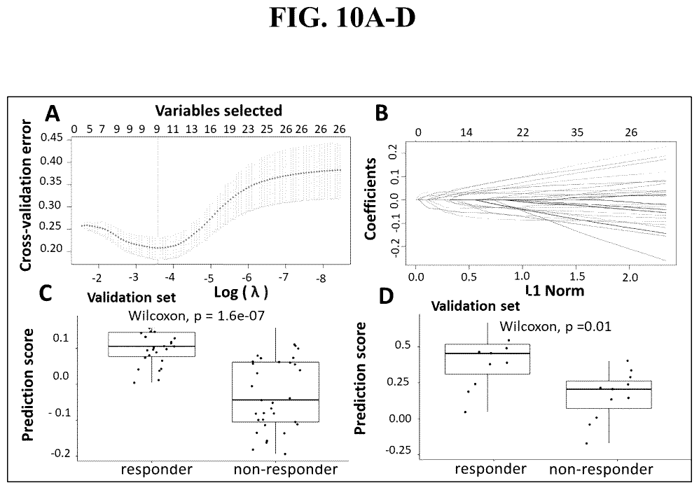

In order to create a predictive model to assess the sensitivity of colorectal cancer patients to FOLFIRI-based chemotherapy, the training dataset included metastatic patients who were divided into two groups: responders and non-responders, depending on their treatment response. The expression values of the selected 26 genes from the training set were then extracted and subjected to analysis using the LASSO regression model. To select an optimal λ, ten-fold cross-validations were performed to calculate the cross-validation error. A dotted vertical line was drawn at the value chosen by 10-fold cross-validation. The optimal λ min value, 0.0281 results in 9 non-zero coefficients (FIG. 10A-B). Thus, the 9-gene signatures selected from 26 DEGs included FLRT3, MIR100HG, LAYN, RUNX1T1, HOXB8, CCN4, DIO2, STYXL2, ABCC13. Furthermore, the prediction scores of the 9-gene signature classifier between responders and non-responders' samples could distinguish the two groups of samples well, both in the training and test sets (FIG. 10C, D). The prediction scores were higher in responders' samples than no responders samples in both sets. These results demonstrated that metastatic samples that differed in response to FOLFIRI-based chemotherapy were more easily distinguished by the 9-gene signature classifier. The prediction score for the signature classifier is calculated using the following formula:

$$Prediction Score = FLRT 3 \times ( - 0.028450931 ) + MIR 100 HG \times 0.012281654 + LAYN \times 0.024434468 + RUNX 1 T 1 \times 0.033506696 + HOXB 8 \times ( - 0.049756588 ) + CCN 4 \times 0.036409569 + DIO 2 \times 0.013353484 + STYXL 2 \times 0.02440122 + ABCC 13 \times ( - 0.042381986 ) \qquad ( 9 )$$

Additionally, the feature selection technique, varSelRF, was utilized on the differentially expressed genes (DEGs). The gene signatures obtained from varSelRF encompassed the following genes: FLRT3, MIR100HG, LAYN, RUNX1T1, HOXB8, CCN4, DIO2, STYXL2, ABCC13. Both methods identified the 9 genes: FLRT3 (Fibronectin Leucine Rich Transmembrane Protein 3), MIR100HG (Mir-100-Let-7a-2-Mir-125b-1 Cluster Host Gene), LAYN (Layilin), RUNX1T1 (RUNX1 Partner Transcriptional Co-Repressor 1), HOXB8 (Homeobox B8), CCN4 (Cellular Communication Network Factor 4), DIO2 (Iodothyronine Deiodinase 2), STYXL2 (Serine/Threonine/Tyrosine Interacting Like 2), ABCC13 (ATP Binding Cassette Subfamily C Member 13 (Pseudogene)). It is noteworthy that the majority of these commonly identified genes are closely associated with colorectal cancer, emphasizing the biological relevance of the models.

After conducting feature selection methods, the gene signatures were progressively evaluated, and the gene set that exhibited the best prediction performance was identified as the optimal gene set. The evaluation of model performance was assessed in training and test sets. The evaluation of model performance was performed in training and independent test sets. As shown in Table 10, the top machine learning algorithm was random forest. These machine learning algorithms were applied to an independent test set to predict FOLFIRI response, and the prediction results were displayed in Table 10. In the independent test set, Random Forest and SVM had an accuracy of 0.96 (95% CI: 0.87, 0.99) and 0.96 (95% CI: 0.87, 0.99) accordingly. In addition, random forest ranked first with a sensitivity of 0.89, specificity of 0.92, and AUC of 0.94. The SVM algorithm was comparable to the random forest algorithm with a sensitivity of 0.89, specificity of 0.93, and AUC of 0.93.

TABLE 10
Comparison of different classification methods on training,
validation, and independent test set after feature
selection using LASSO and VarSelRF method.

| | | mFOLFIRI (LASSO & VarSelRF) | |
|---|---|---|---|
| | | Random | Support Vector |
| Model | | Forest (RF) | Machine (SVM) |
| Training | Accuracy | 1 | 0.96 |
| (n = 60) | 95% CI | (0.94, 1) | (0.84, 0.99) |
| | Sensitivity | 1 | 1 |
| | Specificity | 1 | 0.86 |
| Independent Test | Accuracy | 0.96 | 0.96 |
| (n = 20) | 95% CI | (0.87, 0.99) | (0.87, 0.99) |
| | Sensitivity | 0.89 | 0.89 |
| | Specificity | 0.92 | 0.93 |
| | AUC | 0.94 | 0.93 |

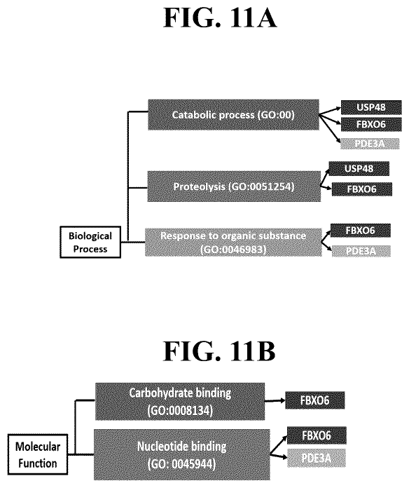

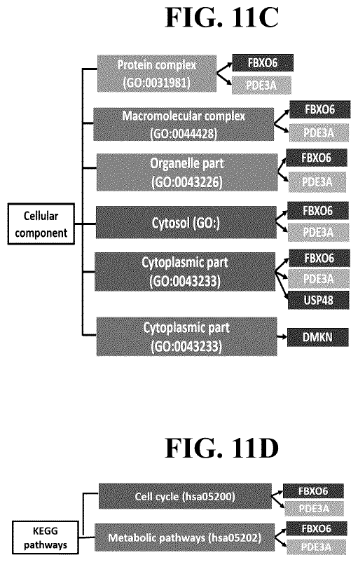

BP, MF, and CC, along with pathways that incorporate these proteins are shown in FIG. 11. The top significant terms from the GO enrichment analysis showed that in the BP category, the gene signatures were involved in regulation of transcription from RNA polymerase promoter II (GO:0006357), negative regulation of RNA metabolite process (GO:0051254), negative regulation of biosynthetic process (GO: 0046983), regulation of gene expression (GO: 0010468), and others. For the MF category, the gene signatures were enriched in DNA binding (GO:0003677), enzyme binding (GO:00453944), and zinc ion binding (GO:0090575). For the CC category, the gene signatures were correlated with nuclear lumen (GO: 0031981), nuclear part (GO:0044428). Organelle lumen (GO:0043233), and others. For KEGG pathway enrichment analysis, among the gene signatures, DIO2 and RUNX1T1 were involved in pathways in cancer (map05200), transcriptional mis-regulation in cancer (hsa05202), and others (FIG. 11).

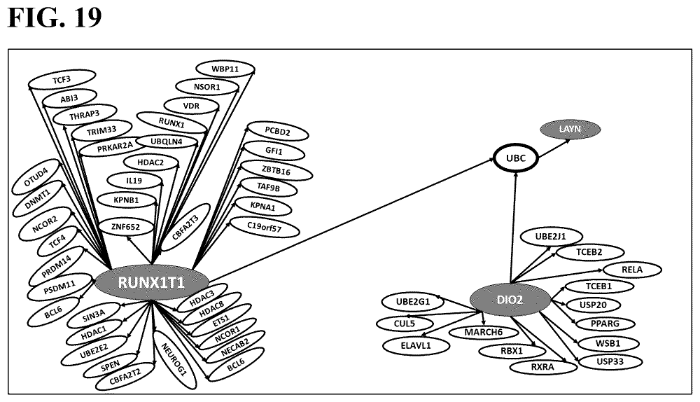

The protein-protein interaction (PPI) networks generated through IMEx indicate (direct and indirect) interactions among these gene encoded proteins (FIG. 19). As shown in FIG. 19, the PPI network comprises 60 nodes and 59 edges with 2 out of 10 genes being hub genes. For instance, DIO2 and RUNX1T1 have the highest number of hub genes. Based on the PPI network predicted using IMEx, the signature proteins have no known direct functional effect on each other. DIO2, RUNX1T1, and LAYN interact directly with UBC.

Moreover, the analysis was broadened to include additional genes that play a critical role in predicting drug response, which were not initially identified through the feature selection methods (LASSO and varSelRF) in the initial phase. Among the 26 genes showing differential expression, 11 genes including CCN4, ABCC13, AKAP12, GLT8D2, LUM, CTHRC1, FLRT3, SFRP2, COL1A2, MIR100HG, and PCDH7 exhibited significant variations in their predictive capacity for determining the response to FOLFOX treatment between individuals who responded positively and those who did not.

Evaluation of the performance of the model utilized both the training and test datasets. The results presented in Table 11 indicate that the random forest algorithm outperformed other machine learning techniques, demonstrating its effectiveness. Subsequently, these algorithms were applied to the validation set to predict the response to FOLFOX treatment, and the prediction results are reported in Table 11. The random forest algorithm achieved an accuracy of 0.91 (95% CI: 0.78, 0.93), while the Support Vector Machine (SVM) algorithm exhibited an accuracy of 0.91 (95% CI: 0.70, 0.92). In addition, random forest ranked first with a sensitivity of 0.82, specificity of 0.85, and AUC of 0.91. The SVM algorithm was comparable to the random forest algorithm with a sensitivity of 0.80, specificity of 0.74, and AUC of 0.91.

TABLE 11
Comparison of different classification methods on training
and validation sets using large panel DEGS.

mFOLFIRI (LASSO & VarSelRF)

| | | Random | Support Vector |
|---|---|---|---|
| Model | | Forest (RF) | Machine (SVM) |
| Training | Accuracy | 0.93 | 0.93 |
| (n = 60) | 95% CI | (0.86, 0.99) | (0.77, 0.95) |
| | Sensitivity | 0.89 | 0.89 |
| | Specificity | 0.87 | 0.87 |
| Validation | Accuracy | 0.91 | 0.91 |
| (n = 20) | 95% CI | (0.78, 0.93) | (0.70, 0.92) |
| | Sensitivity | 0.82 | 0.80 |
| | Specificity | 0.85 | 0.74 |
| | AUC | 0.91 | 0.91 |

### 2.6. Responders Vs Non-Responders Samples Who Received FOLFIRI Responders Vs Non-Responders with Metastatic CRC

The sixth analysis of colorectal cancer patients identified significant genes separating FOLFIRI responders from non-responders for metastatic stages of cancer. The training set, and test set consisted of 21 and 19 CRC patients, respectively, from the datasets GSE62080 and GSE62321 for patients who received first-line FOLFIRI-based treatment. The training set included 21 samples for a total of 12 non-responders and 9 responders of metastatic CRC samples. The independent test data included 19 patients (9 non-responders and 10 responders) from the dataset GSE62321 derived from the platform Affymetrix Human Genome U133B Array (GPL97). Following an integrated bioinformatics analysis, a total of 49 DEGs were identified between pre-chemotherapy tissue samples of non-responders and responders in CRC patients treated with FOLFIRI. Among these genes, 18 were upregulated, while 31 were downregulated.

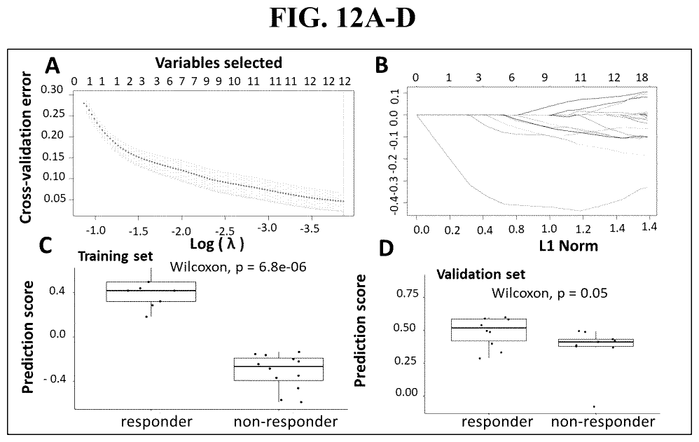

To develop a predictive model for evaluating the sensitivity of colorectal cancer patients to FOLFIRI-based chemotherapy, the training dataset consisted of metastatic patients who were divided into two groups: responders and non-responders based on their treatment response. The expression values of the selected 49 genes from the training set were extracted and analyzed using the LASSO regression model. To select an optimal λ, ten-fold cross-validations were performed to calculate the cross-validation error. A dotted vertical line was drawn at the value chosen by 10-fold cross-validation. The optimal λ min value, 0.0210 results in 13 non-zero coefficients (FIG. 12A-B). Thus, the 13-gene signatures selected from 49 DEGs included DMKN, CPNE5, TNFRS19, ZNF300, USP48, RASGEF1A, PTGR2, SLC51B, FBXO6, PCDHB16, KCNJ3, PDE3A, DNAH14. Furthermore, prediction scores of the 13-gene signature classifier between responders and non-responders samples could distinguish the two groups of samples well, both in the training and test sets (FIG. 12C-D). The prediction scores were higher in responders samples than no responders samples in both sets. These results demonstrated that metastatic samples that differed in response to FOLFIRI-based chemotherapy were more easily distinguished by the 13-gene signature classifier. The prediction score for the signature classifier is calculated using the following formula:

$$Prediction Score = DMKN \times ( - 0.00972630208012686 ) + CPNE 5 \times ( - 0.0020590003928684 ) + TNFRSF 19 \times 0.0875040800143143 + ZNF 300 \times ( - 0.168647678218723 ) + USP 48 \times ( - 0.0555697960359847 ) + RASGEF 1 A \times ( - 0.105383236396517 ) + PTGR 2 \times ( - 0.384234918282503 ) + SLC 51 B \times 0.0626706322652297 + FBXO 6 \times ( - 0.101437610572323 ) + PCDHB 16 \times 0.0567660754509191 + KCNJ 3 \times ( - 0.0808297143523455 ) + PDE 3 A \times 0.0116821827668894 + DNAH 14 \times ( - 0.10533424433337 ) \qquad ( 10 )$$

Furthermore, the feature selection technique, varSelRF, was utilized on the differentially expressed genes (DEGs). The gene signatures obtained from varSelRF encompassed the following genes: DMKN, CPNE5, TNFRS19, USP48, PTGR2, FBXO6, PCDHB16, and PDE3A.

Both methods identified the 8 genes: DMKN (Dermokine), CPNE5 (The copine 5), TNFRS19 (TNF Receptor Superfamily Member 19), USP48 (Ubiquitin-Specific Protease 48), PTGR2 (Prostaglandin Reductase 2), FBXO6 (F-Box Protein 6), PCDHB16 (Protocadherin Beta 16), and PDE3A (Phosphodiesterase 3A). The models identified a set of genes, and it is important to note that most of these genes are strongly linked to colorectal cancer, underscoring the biological significance of the models.

After conducting feature selection methods, the gene signatures were progressively evaluated, and the gene set that exhibited the best prediction performance was identified as the optimal gene set. The evaluation of model performance was assessed in training and test sets. The evaluation of model performance was performed in training and independent test sets. As shown in Table 12, the top machine learning algorithm was random forest. These machine learning algorithms were applied to an independent test set to predict FOLFIRI response, and the prediction results were displayed in Table 12. In the independent test set, Random Forest and SVM had an accuracy of 0.91 (95% CI: 0.81, 0.99) and 0.90 (95% CI: 0.75, 0.96) accordingly. In addition, random forest ranked first with a sensitivity of 0.89, specificity of 0.85, and AUC of 0.91. The SVM algorithm was comparable to the random forest algorithm with a sensitivity of 0.88, specificity of 0.87, and AUC of 0.90.

TABLE 12
Comparison of different classification methods on training,
validation, and independent test set after feature
selection using LASSO and VarSelRF method.

| | | mFOLFIRI (LASSO & VarSelRF) | |
|---|---|---|---|
| | | Random | Support Vector |
| Model | | Forest (RF) | Machine (SVM) |
| Training | Accuracy | 0.94 | 0.92 |
| (n = 21) | 95% CI | (0.82, 0.99) | (0.77, 0.95) |
| | Sensitivity | 0.93 | 0.92 |
| | Specificity | 0.89 | 0.79 |
| Independent test | Accuracy | 0.91 | 0.90 |
| (n = 19) | 95% CI | (0.81, 0.99) | (0.75, 0.96) |
| | Sensitivity | 0.89 | 0.88 |
| | Specificity | 0.85 | 0.87 |
| | AUC | 0.91 | 0.90 |

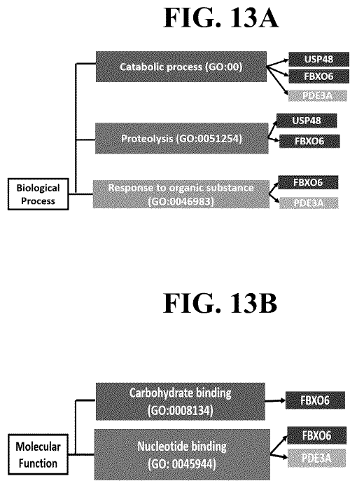

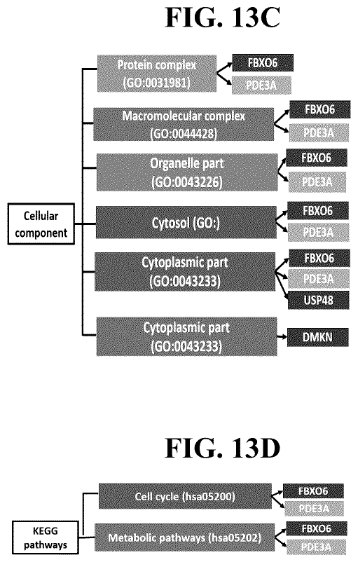

BP, MF, and CC, along with pathways that incorporate these proteins are shown in FIG. 13. The top significant terms from the GO enrichment analysis showed that in the BP category, the gene signatures were involved in catabolic process (GO:009056), proteolysis (GO:0051254), and response to organic substance (GO:0046983). For the MF category, the gene signatures were enriched in carbohydrate building (GO: 0008134). For the CC category, the gene signatures were correlated with protein complex (GO:0031981). Macromolecular complex (GO:0044428), organelle part (GO:0043226), and others. For KEGG pathway enrichment analysis, the gene signatures were enriched in cell cycle (hsa05200) and metabolic pathways (hsa0502) (FIG. 13).

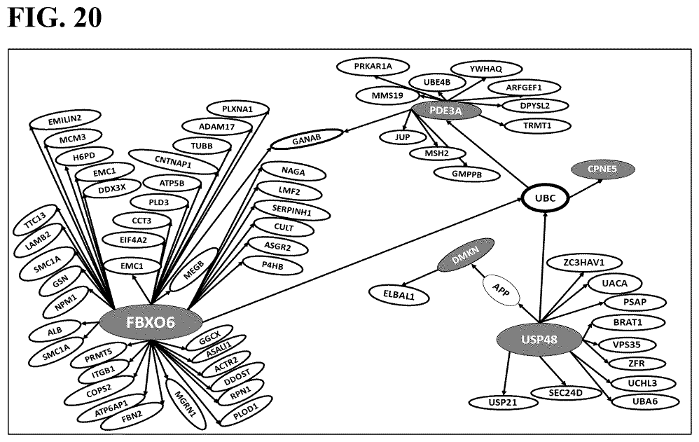

The protein-protein interaction (PPI) networks generated through IMEx indicate (direct and indirect) interactions among these gene encoded proteins (FIG. 20). As shown in FIG. 20, the PPI network comprises 60 nodes and 59 edges with 2 out of 10 genes being hub genes. For instance, DIO2 and RUNX1T1 have the highest number of hub genes. Based on the PPI network predicted using IMEx, the signature proteins have no known direct functional effect on each other. DIO2, RUNX1T1, and LAYN interact directly with UBC.

Furthermore, the analysis was expanded to include additional genes that play a crucial role in predicting drug response. These genes were not initially identified through the feature selection methods (LASSO and varSelRF) in the initial phase. Among the 49 genes that showed differential expression, 33 genes including PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, S1PR3, BTNL9, PCDH7, FNDC1, CDH11, PDE3A, BOC, GDAP1, and FBXO6 displayed significant variations in their predictive ability for determining the response to FOLFOX treatment between individuals who responded positively and those who did not.

The model's performance was assessed using both the training and validation datasets. According to the results displayed in Table 13, the random forest algorithm proved to be the most effective machine learning technique. Subsequently, these algorithms were applied to the validation set to predict the response to FOLFOX treatment, and the prediction outcomes were presented in Table 13. The random forest algorithm achieved an accuracy of 0.92 (95% CI: 0.89, 0.97), while the Support Vector Machine (SVM) algorithm exhibited an accuracy of 0.92 (95% CI: 0.74, 0.93).

TABLE 13
Comparison of different classification methods on training
and validation sets large panel of significant genes.

mFOLFIRI (LASSO & VarSelRF)

| | | Random | Support Vector |
|---|---|---|---|
| Model | | Forest (RF) | Machine (SVM) |
| Training | Accuracy | 0.94 | 0.93 |
| (n = 21) | 95% CI | (0.86, 0.96) | (0.79, 0.94) |
| | Sensitivity | 0.92 | 0.92 |
| | Specificity | 0.88 | 0.83 |
| Independent | Accuracy | 0.92 | 0.92 |
| test set | 95% CI | (0.89, 0.97) | (0.74, 0.93) |
| (n = 19) | Sensitivity | 0.91 | 0.89 |
| | Specificity | 0.89 | 0.76 |
| | AUC | 0.92 | 0.92 |

### 2.7. Machine Learning Model Application to Predict Effectiveness of Alternate Chemotherapy Regimen

In the analysis above, the genes that successfully classified responders and non-responders for FOLFOX differed from the genes that successfully classified responders and non-responders for FOLFIRI except for one gene that was present in both, namely secreted frizzled related protein 2 (SFRP2). This suggests that there might be different underlying mechanisms involved (consistent with the two therapies differing in cellular targets) and, consequently, patients who did not respond to FOLFOX might respond to FOLFIRI and vice versa. When the Random Forest model for the FOLFIRI data set was applied to the prediction of cases of colon cancer treated with the FOLFOX regimen, the results show that 25 of 56 (44.6%) metastasis CRC patients who did not respond to FOLFOX would respond to FOLFIRI and that 20 of 74 samples (27.02%) of primary CRC patients who did not respond to FOLFOX are predicted to respond to FOLFIRI (Table 6). When the FOLFOX training model for metastatic CRC was applied to the prediction of cases of colon cancer samples treated with the FOLFIRI regimen, the results showed 25 of 81 (30.9%) patients who did not respond to FOLFIRI would respond to FOLFOX. Applying the FOLFOX training model for primary CRC to the FOLFIRI cases, 5 of 81 (6.2%) patients who did not respond to FOLFIRI are predicted to respond to FOLFOX. Assuming 94% accuracy for the FOLFOX model and 96% accuracy for the FOLFIRI model, a Chi-squared test shows that these results are significant at the p>0.00001 level. This analysis predicts that it is likely that 28.6% of patients on average that failed one drug treatment regimen would have responded to the other treatment regimen. However, further clinical validation would be needed before this could influence clinical care.

TABLE 14
Prediction of alternative therapy efficacy

| | FOLFOX responder | FOLFOX non-responder |
|---|---|---|
| | (metastasis) | (metastasis) |
| Responder with FOLFIRI model | 8 (14.3%) | 25 (44.6%) |
| Non-responder with FOLFIRI model | 23 (41.8%) | 0 (0.0%) |
| | FOLFOX responder | FOLFOX non-responder |
| | (Primary) | (Primary) |
| Responder with FOLFIRI model | 15 (20.27%) | 20 (27.02%) |
| Non-responder with FOLFIRI model | 17 (23%) | 22 (28.9%) |

| | FOLFIRI responder | FOLFIRI non-responder |
|---|---|---|
| Responder with FOLFOX | 12 (14.8%) | 25 (30.09%) |
| (metastasis) model | | |
| Non-responder with FOLFOX | 24 (29.6%) | 20 (24.7%) |
| (metastasis) model | | |
| Responder with FOLFOX | 19 (23.4%) | 5 (6.2%) |
| (primary) model | | |
| Non-responder with FOLFOX | 26 (32.09%) | 40 (49.3%) |
| (primary) model | | |

### 2.7. Prognostic Value of Gene Panels

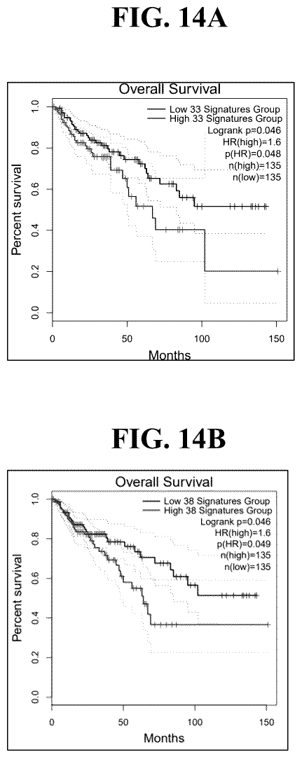

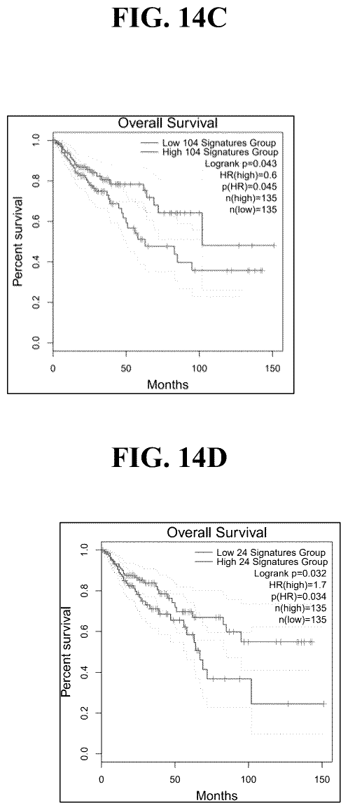

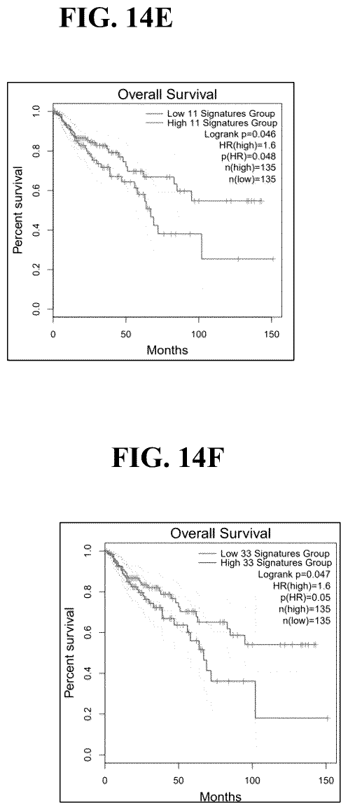

Finally, the study examined whether the gene signature identified for the early-stage and metastatic CRC cancer patients undergoing FOLFOX or FOLFIRI treatment could serve as a prognostic indicator for overall survival (OS) in patients from The Cancer Genome Atlas Colon Adenocarcinoma (TCGA-COAD) dataset with the construction of Kaplan-Meier survival analysis using the GEPIA2 platform. The gene signatures varied for the different cases. The patients were divided into low-risk and high-risk groups, each consisting of 135 patients, based on the median expression value of the gene signature, which included the genes identified through the feature selection methods. The GEPIA2 analysis demonstrated that the overall survival (OS) of patients with downregulation of the 33 genes identified in CRC patients in all stages treated with FOLFOX are predicted to be more than 40 months longer than OS of patients treated counter to the prediction (p-value=0.046), as demonstrated in FIG. 14A. In FIG. 14B, the GEPIA2 analysis showed that the overall survival (OS) of patients with downregulation of 38 genes identified in the early-stage CRC patients treated with FOLFOX are predicted to be more than 40 months longer than the OS of patients treated counter to the prediction (p-value=0.046), as illustrated in FIG. 14B. In FIG. 14C, the GEPIA2 analysis demonstrated that the overall survival (OS) of patients with upregulation of the 104 genes identified in the metastatic CRC patients treated with FOLFOX are predicted to be more than 40 months longer than the OS of patients treated counter to the prediction (p-value=0.043). The GEPIA2 analysis demonstrated that the overall survival (OS) of patients with downregulation of the 24 genes identified in all stages of CRC patients treated with FOLFIRI are predicted to be more than 80 months longer than the OS of patients treated with counter to the prediction (p-value=0.032) as illustrated in FIG. 14D. In FIG. 14E, GEPIA2 analysis demonstrated that the overall survival (OS) of patients with upregulation of the 11 genes identified in the early-stage CRC patients treated with FOLFIRI are predicted to be more than 80 months longer than the OS of patients treated counter to the prediction (p-value=0.046), as illustrated in FIG. 14E. In FIG. 14F, the GEPIA2 analysis demonstrated that the overall survival (OS) of patients with upregulation of the 33 genes identified in the metastatic CRC patients treated with FOLFIRI are predicted to be more than 80 months longer than the OS of patients treated counter to the prediction (p-value=0.047). In conclusion, both the Kaplan-Meier survival analysis and the area under the curve (AUC) values provided evidence that the identified gene signatures exhibit a high level of prognostic accuracy in stratifying colorectal cancer patients based on their survival outcomes. These findings further validate the effectiveness of this prognostic gene signature.

FOLFOX and FOLFIRI are combination chemotherapies that have been used as a first-line treatment for patients with late-stage colon cancer. Previous studies have shown FOLFOX and FOLFIRI to be ˜52% and ˜39% effective, respectively [49, 50]. Though these regimens can significantly extend the median overall survival up to 15 months, many individuals do not achieve long-term clinical benefit with a given treatment [51]. Since these therapies target different cell mechanisms, there is the possibility that the actual responders may be different between the two drugs. Thus, improving methods of identifying patients who would respond better to these drugs would help oncologists determine optimum treatment regimens for their patients.

Gene-expression profiles have the potential to predict cancer patient outcome and drug response in comparison to the conventional clinical and pathological techniques [24, 52-55]. In contrast to the numerous studies to identify the estimation of responders to anticancer drugs using expression profiling in other cancer types such breast and ovarian cancer, only a few such studies have been conducted in colorectal cancer [18, 20, 24, 56]. A direct comparison with a previously published machine learning model on the same dataset indicates that the performance of the models presented in this paper is superior in predicting FOLFOX and/or FOLFIRI drug response. Tsuji and co-workers identified a signature consisting of 14 genes using random forest embedded selection that was able to predict FOLFOX responders in a sample size of 83 patients [20]. Using these genes, RF classifier was able to correctly classify 21 of 23 responders (91.3%) and 22 of 23 non-responders (95.6%) in the training set, with an accuracy of 69.2% in 29 independent test samples. Also, an older study by Del Rio and co-workers identified 14 genes for predicting response to FOLFIRI, although it included only 21 patients [24].

Previous studies that have used expression data to predict FOLFOX or FOLFIRI efficacy have produced AUC in the range of 0.66-0.92 [16, 18, 19, 21]. The model prediction had AUC in the range of 0.87-1. There are several reasons why these results were better. First, in the method presented here, data cleansing and GCRMA were applied rather than mass5 or RNA to remove noise. While this reduced the size of the data set, it also improved accuracy. Second, the use of two types of feature selection LASSO and VarSeIRF improved accuracy while the other studies did not use any feature selection or used only LASSO feature selection. Third, splitting the data into the different stages of cancer improved the results. Other studies failed to do so, possibly to make the data set as large as possible. Finally, the implementation of these steps resulted in no outliers being found when doing outlier analysis. The outliers have the tendency to reduce the model performance.

The purpose of this study was to identify gene signatures that could predict the response to FOLFOX and FOLFIRI in patients with early stage and metastatic CRC. To determine the gene signature for response prediction from gene expression profiling, significant differentially expressed genes (DEGs) were first selected. The DEGs were filtered using the variable selection methods including LASSO and varSelRF. The performance of the models was evaluated using two machine learning classifiers, RF and SVM. Overall, the machine learning model with enhanced feature selection achieved 94-96% accuracy for predicting the response of patients to FOLFOX or FOLFIRI using retrospective cancer patient data available in public datasets. These results held for data sets that were not part of the training data. Furthermore, for those patients that did not respond to FOLFOX, 35% are predicted as FOLFIRI responders and for those patients that did not respond to FOLFIRI 18% are predicted as FOLFOX responders. This suggests that the biomarkers identified here can help select which chemotherapy regimen to use on patients after additional validation studies.

### 4.1. Gene Signatures Identified in FOLFOX Responders Vs Non-Responders in all Stages of CRC

Through integrated bioinformatics analysis, 10 gene signatures were identified in FOLFOX responders vs non-responders in all stages of CRC, among them, the hub genes, consisting of 5 genes, including CARM1, LTA4H, GTF2A1, TRIM3, and SH3GLB1, were listed as the genes with many connections to other genes (FIG. 15), and their biological functions are involved in regulation of transcription from RNA polymerase II promoter, regulation of cellular metabolic process, regulation of gene expression, and regulation of the apoptotic process (FIG. 2). Previous studies reported that the expression of CARM1 and TRIM3 is linked to NF-kB response by stimulation of TNF-α and the p53 response[57-59].

CARM1 (Coactivator-associated arginine methyltransferase 1), also known as PRMT4, acts as a transcriptional coactivator for several different types of DNA-binding transcriptional activator proteins, and thus deregulated CARM1 expression likely to affect many transcriptional programs which target genes that control proliferation rate or other oncogenic properties [60-63]. In fact, studies have shown that CARM1 depletion causes reduced expression of genes involved in Wnt-driven tumorigenesis (e.g., c-myc[64]), metastasis (e.g., S100A4[65]), and prevention of apoptosis (e.g., GRP49[66]). Wnt/β-catenin activation and malignant transformation of inflammatory bowel illness have been identified as the two leading causes of colorectal cancer [67, 68]. Activation of the Wnt/β-catenin and inflammatory signaling pathways disrupts intestinal epithelial homeostasis, resulting in increased proliferation, decreased differentiation, and decreased apoptosis [69]. CARM1 plays a crucial role in Wnt signaling through its function as a transcriptional co-activator that mediates the actions of β-catenin on Wnt target genes. Another study reported that inhibition of CARM1-mediated MED12 methylation promotes downregulation of MED12 and a subsequent upregulation of P21/WAF1 expression to drive chemotherapy resistance [70-73]. Therefore, selective inhibition of CARM1 methyltransferase activity or CARM1 binding to β-catenin may be a potential strategy for therapeutic treatment of abnormally activated Wnt/β-catenin signaling in colorectal cancers.

TRIM3, a member of the TRIM protein superfamily, has been reported as tumor suppressor in different cancers, including colorectal cancer. It controls cell proliferation, migration, and invasion of cancer cells by enhancing p53 stability, and simultaneously stimulating transcriptional activity of downstream target genes, p21 and GADD45[74]. TRIM3 can also directly interact with p21, preventing from binding to cyclin D1-cdk4 and eventually reducing proliferation [75]. Furthermore, it has been shown that TRIM3 inactivates the p38 MAPK pathway, which has negative effects on cell proliferation [76]. However, the results of the inactivation of p38 signaling pathway depend significantly on the cellular environment, and more specifically on the presence of a mutated or wildtype p53. In the former, TRIM3 action contributes to chemoresistance to DNA-damaging drugs by suppressing apoptosis, whereas in the latter, it can suppress cell proliferation increasing the response to the chemotherapeutic agent [77, 78].

SH3GLB1 (SH3 domain GRB2-like endophilin B1), also known as Bif-1 and endophilin B1, is a tumor suppressor gene of the endophilin protein family [79]. SH3GLB1 interacts with BAX to regulate apoptosis [80]. Inhibition of SH3GLB1 suppresses apoptotic cell death by inhibiting BAX-BAK1 conformational change and caspase activation [81]. Reduced expression of Bax was correlated with poor differentiation, metastatic progression, and is a negative prognostic factor in patients with CRC[82, 83]. SH3GLB1 also regulates the induction of autophagy [84]. SH3GLB1 colocalizes with ATG5 and LC3, which suggests its involvement in early autophagosome formation. A study reported that one of the LC3 isoforms, LC3-II, is overexpressed in CRC cells particularly in advanced stages, compared to normal colon cells [85]. ATG5 is downregulated in 95% of CRC cases, according to the same study, and that plays a major role in CRC progression and chemotherapy resistance [85]. Furthermore, during autophagy, SH3GLB1 interacts with BECN1 (beclin-1) through UVRAG (UV radiation resistance associated), and it is a positive regulator of PI3KC3 (class III PI3-kinase), resulting in the induction of autophagy of cells [83, 84]. However, Beclin-1 has a debatable role in CRC in that it promotes tumorigenesis but may paradoxically inhibit cell growth. Increased BECN1 expression was associated with better overall survival (OS) in patients with locally advanced colon carcinomas who received postoperative 5-FU chemotherapy for 6 months [86]. Overexpression of BECN1 in cases with resected stage II and III colon carcinomas, who received 5-FU based chemotherapy was associated with worse OS, denoting a potential effect of autophagy in drug resistance [87].

LTA4H (leukotriene A4 hydrolase) is an epoxide hydrolase that catalyzes conversion of the unstable allelic epoxide LTA4 to leukotriene B4 (LTB4). LTA4H is overexpressed in several cancers including CRC, and several studies have shown that its hydrolase function is implicated in cancer development [88-90]. LTA4H is a key modulator of the cell cycle through its negative effect on the expression of the tumor suppressor p27 protein [91]. The Cyclin-dependent kinase inhibitor 1B (CDKN1B, p27Kip1), known as p27 protein, controls the transition from the G1 phase into the S phase of the cell cycle [92]. The inactivation of p27 is generally accomplished post-transcriptionally by the oncogenic activation of various pathways that accelerate the proteolysis of the p27 protein and allow cancer cells to undergo rapid division and uncontrolled proliferation [88, 92, 93]. The depletion of LTA4H enhances p27 protein stability by mediating the downregulation of its ubiquitination. This eventually leads to a decrease in cancer cell growth by inducing cell cycle arrest at the G0/G1 phase [91]. Taken together, this suggests that inhibiting LTA4H epoxide hydrolase activity is a promising strategy for cancer prevention.

GTF2A1, general transcription factor IIA subunit 1, has never been associated with colorectal cancer.

### 4.2. Gene Signatures Identified in FOLFOX Responders Vs Non-Responders in Early Stages of CRC

Further analysis on FOLFOX therapeutic response prediction in various stages of CRC showed 10 genes associated with early-stage CRC. Among the 10 genes related to early-stage CRC, HOXA10, HOXA11, NR4A1, and CYP2B6 were identified as the hub genes in the protein-protein interactions network (FIG. 16). The biological functions of these genes are involved in regulation of transcription from RNA polymerase II promoter, regulation of cellular metabolic process, regulation of gene expression, regulation of cell differentiation, and response to chemical stimulus (FIG. 5).

Nuclear receptor subfamily 4 group A member 1 (NR4A1) is an immediate-early response gene that responds rapidly to various factors such as hormones, growth factors, and inflammatory factors. Numerous studies suggest that NR4A1 can function as either an oncogene or a tumor suppressor. It acts as an oncogene in lung cancer, breast cancer, and colorectal cancer[94], while exhibiting tumor suppressor properties in acute myeloid leukemia or metastatic ovarian cancer. NR4A1 is up regulated in colorectal cancer tissues and cells. It plays a role in promoting metastasis and invasion through various mechanisms such as regulating the MMP9/E-cadherin axis[94], β1-integrin [95], or forming a feed-forward loop with β-catenin in hypoxic conditions [96]. Lee and colleagues demonstrated that silencing NR4A1 resulted in the inhibition of mTOR signaling, reduced surviving expression, and suppressed growth of colon cancer cells. Similarly, the study also observed up-regulation of NR4A1 expression in CRC samples. Due to its oncogenic properties observed in various cancers, NR4A1 holds promise as a potential therapeutic target for clinical use. Research has indicated that diindolylmethane analogs (C-DIMs) specifically target nuclear NR4A1, triggering apoptosis in cancer cells and tumors[97].

CYP2B6 codes for an enzyme belonging to the cytochrome P450 superfamily, a group of enzymes that play a crucial role in tumor development by metabolizing various carcinogens[98]. Immunohistochemistry studies have identified several cytochrome P450 enzymes with increased expression in colorectal cancer (CRC), suggesting their potential as independent prognostic markers[99]. In addition, CYP2B6 plays a significant role in human drug metabolism[100]. Variations in the expression and function of CYP2B6 significantly alter the metabolism and pharmacokinetics of many drugs. These alterations may result in significant drug-drug interactions which may lead to improved therapy or toxicity[101].

Transient receptor potential melastatin-4 channel (TRPM4) is expressed in several human tissues and has been identified as a cancer driver gene that contributes to migration, proliferation, and invasion of prostate cancer (PCa) cells[102]. It has been reported the mechanisms of action for TRPM4 including alteration of Ca2+ signaling, since Na+ influx via TRPM4 can reduce the driving force for Ca2+ entry and alteration of the APC pathway[103-105]. Alterations in the APC-WNT/β-catenin, transforming growth factor-β, EGFR, and downstream MAPK and PI3K signaling pathways are nearly ubiquitous events in CRC. Study reported that TRPM4 protein levels were highly expressed, while mRNA levels were rather low, suggesting that it could be due to post-translational mechanisms that are altered in cancer cells and affect TRPM4 function, for example, glycosylation[106].

HOXA10, a member of HOX genes, is involved in regulating differentiation and progression in several cancer types, including CRC [107, 108]. knockdown of HOXA10 in CRC cells promoted cell migration and invasion and its re-expression induces loss of the cancer stem cell phenotype, thereby preventing tumor progression and metastasis[108].

The expression of HOXA11-AS (homeobox A11 antisense RNA), a lncRNA, has been found in diverse human neoplasms, including CRC [109]. In vitro and in vivo assays evaluating the effects of HOXA11-AS alterations revealed a complex integrated phenotype affecting cell growth, apoptosis, migration, invasion and stemness maintenance through multiple biologic processes, such as epithelial-mesenchymal transition (EMT) [110-112]. Recently, HOXA11-AS has been reported as a tumor-suppressor gene or an oncogene in two independent CRC-relevant studies. In the first study, the author found that HOXA11-AS was decreased in CRC tissues and cell lines. Clinicopathologic analysis further proved that HOXA11-AS downregulation was significantly related with CRC patients' tumor size, lymph node metastasis, TNM stage and carcinoembryonic antigen level, which indicated HOXA11-AS to be a tumor-suppressor gene in CRC [109]. Conversely, in the other study, HOXA11-AS was identified as a highly related oncogene to liver metastasis in CRC. In more detail, HOXA11-AS was significantly upregulated in 15 patients with liver metastasis and highly invasive cell lines; gain-/loss-of-function studies showed that HOXA11-AS promoted CRC cell migration and invasion [113]. The different conclusions of these two studies may be attributed to the differences in the patients' samples collected and the selected cell types.

### 4.3. Gene Signatures Identified in FOLFOX Responders Vs Non-Responders in Metastatic CRC

Among the 12 genes related to metastatic, IQGAP2 and EEF1D were identified as the key genes in the protein-protein interactions network (Figure S3). The biological functions of these genes are involved in innate immune response, defense response to virus, cytokine mediated signaling pathway, and response to chemical stimulus (FIG. 7).

IQ motif-containing GTPase-activating proteins (IQGAPs) are a group of proteins, namely IQGAP1, IQGAP2, and IQGAP3, that are evolutionary conserved. These proteins share similar structural domains and sequence similarity. The complex structure of IQGAPs enables the formation of protein complexes that are essential for various cellular functions [114, 115]. Although understanding of IQGAP2 and IQGAP3 is limited compared to IQGAP1, they have been found to play crucial roles in the progression of cancer, particularly in various malignancies[116]. Researchers have observed that IQGAP2 and IQGAP3 have opposing functions in gastric cancer, colorectal cancer, hepatocellular carcinoma, prostate cancer, ovarian cancer, breast cancer, and malignant lymphoma [117-122]. A poorer prognosis is associated with lower levels of IQGAP2 expression or higher levels of IQGAP3 expression. Two studies have investigated the relationship between IQGAP2 and colorectal cancer. The first study demonstrated a decrease in IQGAP2 at both the mRNA and protein levels in colorectal cancer tissue, but no significant correlation with overall survival was observed [123]. In contrast, the second study found that IQGAP2 was overexpressed in colorectal cancer tissues. These two publications revealed how IQGAP2 was regulated. Overexpression of miR-92a and miR-29a-3p was found to negatively regulate IQGAP2 in colorectal cancer cell lines [124, 125]. Considering that dysregulation of the Wnt signaling pathway is crucial for colorectal cancer development, as nearly all colorectal cancers exhibit abnormalities in this pathway, further research is needed to determine if IQGAP2 plays a role in colorectal cancer carcinogenesis through the Wnt pathway [126, 127].

EEF1D, as a part of the eukaryotic translation elongation factor 1 (EEF1) complex, serves as the enzymatic delivery of aminoacyl tRNAs to the ribosome and functions as a guanine nucleotide exchange factor. Based on the canonical and noncanonical functions, emerging amounts of evidence indicate that EEF1 proteins, particularly the prototypical member EERD, may play a role in the control of cellular processes during tumorigenesis [128, 129]. The overexpression of EEFD has been identified in many cancers including colorectal cancer [130]. One study confirmed that a high EEF1D mRNA level is associated with lymph node metastasis, advanced stage, and shorter disease-specific survival in patients with esophageal cancer [131]. In addition, it has been reported that EEF1D promotes tumor cell proliferation through the Rb-E2F pathway, Akt-mTOR and Akt-Bad pathways, and promotes tumor migration and invasion by influencing EMT process [132].

### 4.4. Gene Signatures Identified in FOLFIRI Responders Vs Non-Responders in Metastatic CRC

A similar analysis performed on FOLFIRI therapeutic response prediction in CRC patients showed 12 gene signatures associated with CRC. Among these 12 genes, CD36, COL1A2, COL3A1, AKAP12, and AMOTL1 were identified as hub genes in the protein-protein interactions network (Figure S4). The biological functions of these genes are involved in cell differentiation and migration, tissue development, regulation of signal transduction, and response chemical stimulus (FIG. 9).

CD36 (Fatty acid translocase) plays a significant role in dietary fatty acid regulation as an exogenous fatty acid transporter [133]. A previous study found that colorectal cancer (CRC) cells with a higher metastatic potential express a higher level of fatty acid translocase (CD36). The same study revealed that high expression of CD36 promotes invasion of CRC cells. The author demonstrated that upregulation of CD36 expression promotes the metastatic properties of CRC via upregulation of MMP28 and an increase in E-cadherin cleavage, suggesting that targeting the CD36-MMP28 axis may be an effective therapeutic strategy for CRC metastasis [134].

Type I collagen is a heterotrimeric protein consisting of two al chains (COL1A1) and one α2 chain (COL1A2). COL1A2 has been found to play a prognostic role in various cancers [135]. COL1A2 mRNA expression in CRC tissues was reported to be positively correlated with tumor differentiation, invasion depth, and lymph node metastasis [136, 137]. A recent study found that COL1A2 overexpression inhibited CRC cell proliferation, migration, and invasion by regulating multiple cancer-associated pathways such as protein kinase A signaling, HGF signaling, glioblastoma multiforme signaling, ephrin B signaling, IL-8 signaling, and NF-κB signaling. Thus, these findings suggest an anticarcinogenic role of COL1A2 in CRC development, which is consistent with studies highlighting the tumor-suppressive role of COL1A2 in certain malignancies [135].

Collagen type III alpha 1 (COL3A1), one of the members of collagen family is mainly expressed in extensible connective tissues including skin and vessels. COL3A1 was found to be upregulated in several cancers [138, 139]. The upregulation of COL3A1 transcription was shown in colorectal cancers comparing with the normal counterparts by microarray gene expression analyses and RNA-seq technique. COL3A1 transcription level was increased from adenoma to carcinoma, indicating an involvement of COL3A1 in carcinogenesis [140]. Importantly, COL3A1 was found to be substantially overexpressed in the liver invasion front of the colorectal liver metastases comparing with the tumor center and the normal tissue, suggesting a potential role of this gene in the metastasis process [141].

AKAP12 (A-kinase anchor protein 12), belonging to the kinase scaffolding protein family, performs its function by anchoring protein kinase A and protein kinase C to the plasma membrane [142]. Recently, the divergent functions of AKAP12 in cancers have been studied extensively. AKAP12 is recognized as a tumor suppressor, whose diminished expression in cancer cells is accompanied by an enhanced invasive and metastatic phenotype [143]. A recent study demonstrated that AKAP12, which is epigenetically regulated by HDAC3, is a suppressive regulator with the capability to inhibit cell growth and migration and promote the apoptosis of CRC cells. Moreover, the downregulation of AKAP12 by HDAC3 is indispensable for HDAC3-induced PI3K/AKT activation and consequent cell metastasis [143].

Angiomotin (AMOT) was initially identified as an angiostatin-binding protein and belongs to the motin family, which includes AMOT (p80 and p130 isoforms), AMOT-like protein 1 (AMOTL1) and AMOTL2 [144]. Previous studies have suggested that AMOT can enhance endothelial cell motility and tube formation, implying a critical role in angiogenesis [144, 145]. Recently, increasing attention has shed light on the role of AMOT in the pathogenesis of cancer. the higher expression of AMOT was detected in CRC cells. overexpression of AMOT promoted LoVo cell proliferation and resistance to 5-FU-induced apoptosis. Moreover, its upregulation also promoted the cell growth and metastatic potential in CRC cells mainly by activating the YAP-ERK/AKT signaling pathway. Blocking AMOT expression inhibited CRC cell growth and metastatic potential. Therefore, these findings suggest that AMOT may elicit the oncogenic function in the carcinogenesis of CRC [146].

### 4.5. Gene Signatures Identified in FOLFIRI Responders Vs Non-Responders at Early Stages CRC

A similar analysis performed on FOLFIRI therapeutic response prediction in CRC patients showed 12 gene signatures associated with CRC. Nine genes, FLRT3, MIR100HG, LAYN, RUNX1T1, HOXB8, DIO2, STYXL2, ABCC13 and CCN4 were identified as gene signatures. Among these 9 genes, RUNX1T1 and DIO2 were identified as hub genes in the protein-protein interactions network (FIG. 19).

A previous study demonstrated that Stromal iodothyronine deiodinase 2 (DIO2) plays a role in promoting the development of intestinal tumors in Apc Δ716 mice. Moreover, in situ hybridization analysis data provided in the study indicated the increased expression of DIO2 specifically in the stromal component of CRC tissues[147]. Other studies have reported both tumor-promoting and tumor-suppressive effects of thyroid hormone (TH) signaling in CRC, but these studies primarily focused on TH signaling in cancer epithelial cells rather than in the tumor stromal cells. Specifically, TRα1 was shown to activate Wnt signaling in intestinal epithelial cells by directly inducing the transcription of the CTNNB1 gene, which encodes β-catenin, and the sFRP2 gene, which encodes a Frizzled-related protein[148-151][152]

Runt-related transcription factor 1 (RUNX1T1) act as an oncogene or as a tumor-suppressor gene [153, 154]. However, few studies have revealed the existence of RUNX1T1 mutations in various solid tumors, including CRC. Hence the role of RUNX1T1 in tumor progression, particularly CRC, is currently not well understood. A Study showed that the overexpression of RUNX1T1 reduce the proliferation of HCT116 CRC cells and increased the sensitivity of CRC cells to 5-FU[155]. In addition, the pathway analysis of genes down-regulated in RUNX1T1-transduced HCT116 cells revealed DNA-damage response among the most affected pathway, suggesting that overexpression of RUNX1T1 might sensitize cancer cells to DNA damage-inducing agents [155].

### 4.6. Gene Signatures Identified in FOLFIRI Responders Vs Non-Responders in Metastatic CRC

Finally, bioinformatics analysis performed on FOLFIRI therapeutic response prediction in metastatic CRC patients showed 8 gene signatures associated with CRC. Eight genes, DMKN, CPNE5, TNFRS19, USP48, PTGR2, PDE3A, FBXO6, and PCDHB16 were identified as gene signatures. Among these 8 genes, FBXO6, USP48, and PDE3A were identified as hub genes in the protein-protein interactions network (FIG. 20).

Multiple studies have reported on the role of USP48, specifically its deubiquitinating activity, in the regulation of NFκB signaling. NFκB signaling plays a crucial role in various cellular processes, including inflammation, immunity, differentiation, and cell survival. In relation to cancer, USP48 has been associated with glioblastoma tumorigenesis by stabilizing Gli1. It has also been found to stabilize the oncoprotein Mdm2 in a manner independent of deubiquitylation and decrease the functionality of E-cadherin-mediated adherens junctions. Apart from its oncogenic functions, USP48 contributes to genome stability by counteracting the E3 ligase function of BRCA1. Additionally, it aids in the granulocytic differentiation of acute promyelocytic leukemia cells induced by ATRA, thereby serving as a tumor suppressor. However, the abundance and biological activity of USP48 in CRC remain entirely unknown[156-159].

It has been demonstrated that the overexpression of PDE3A plays a role in activating the NFκB inflammatory signaling pathway, as well as promoting the transcription of the stemness gene OCT4. It has also been found to facilitate the nuclear translocation of CCDC88A, thereby promoting invasion and metastasis. A recent study has indicated that the activity of the KIT receptor can influence the expression of PDE3A in human gastrointestinal tumors through the MAPK/ERK pathway, both at the transcriptional and protein levels. Additionally, another study has discovered that PDE3A is subject to hypermethylation in cisplatin-resistant non-small cell lung cancer and acts as a modulator of chemotherapy responses. Consequently, PDE3A has the potential to activate multiple pathways, including the inflammatory signaling pathway or CCDC88A[160-164].

FBXO6 has been identified as a potential biomarker for predicting the responsiveness of anticancer drugs. In human breast tumor tissues, an inverse correlation was observed between FBXO6 and checkpoint kinase 1 (Chk1), a key component involved in the replication checkpoint response to DNA damage[165, 166]. Zhang et al. proposed that FBXO6 facilitates the degradation of Chk1, and a deficiency in this process may contribute to increased resistance of tumor cells to specific anticancer drugs like CPT. The study further suggested that enforced expression of FBXO6, but not Skp2, in CPT-resistant cells leads to the degradation of endogenous Chk1 levels. Consequently, these cells exhibited a strong staining pattern for the cleavage product of caspase-3 following CPT treatment. Conversely, depletion of FBXO6 reduced the sensitivity of the lung cancer cell line A549 to CPT, and this effect was completely reversed by depletion of Chk1[165, 166].

### 4.7. Prognostic Value of Identified Gene Values

Prognostic prediction of a cancer is very important for the clinical management of a patient's cancer [167]. Traditionally most cancer staging has been based on anatomic pathology [167]. Defining molecular signatures or gene panels can help further granularize and personalize the cancer treatment strategy for a specific patient [167, 168]. Fang and colleagues used public gene expression data (TCGA GSE44861, and GSE44076 datasets) from colorectal cancer to define 12 gene panel (ADORA3, CPA3, CPM, EDN3, FCRL2, MFNG, NAT1, PCSK5, PPARGC1A, PRRX2, TNFRSF17, and WDR78) as a prognostic panel that showed modest survival benefit between the low risk and high-risk groups in the validation sets. Dalerba and co-workers and Hansen and co-workers found that lack of CDX2 expression in stage II CRC was a prognostic maker of the benefit from adjuvant chemotherapy with an increased overall survival of more than 24 months [169, 170]. Zhang and colleagues studied the use of COL10A1, COL1A1, COL1A2, COL3A1, COL4A1, COL5A2, and COL6A3 expression as a measure of CRC prognosis and suggested that COL1A1 and COL4A1 were associated with an increase in overall survival [171]. Lu and co-workers used machine learning to determine that expression levels of WASHC4, HELZ, ERN1, RPS6KB1, and APPBP2 were downregulated, and expression levels of IRF7, EML3, LYPLA2, DRAP1, RNH1, PKP3, TSPAN17, LSS, MLKL, PPP1R7, GCDH, C19ORF24, and CCDC124 were upregulated in FOLFOX responders vs non-responders [18]. Abraham and co-workers used AI to find a 67-gene panel tested on two independent data sets (from patient data and Phase III TRIBE2 study data) to demonstrated that an increase of 17.5 months in overall survival (OS) of patients treated in a manner consistent with the identified gene signature [172]. These and other studies have shown that a gene signature can be of prognostic value. The panels identified in this study suggest a large increase in overall survival for patients with the gene signatures. This prediction can be tested in further clinical studies to evaluate their potential as a prognostic value panel for CRC.

TABLE 15
Differentially Expressed Genes for FOLFOX (All Stages of CRC)

| | logFC | Log2FC | AveExpr | t | P. Value | FDR | B | gene.symbols |
|---|---|---|---|---|---|---|---|---|
| 203285_s_at | 0.646825 | 1.565719 | 8.5056 | 4.922877 | 2.52E−06 | 0.000248 | 4.388608 | HS2ST1 |
| 222574_s_at | 0.602011 | 1.517831 | 8.717102 | 4.917414 | 2.58E−06 | 0.000248 | 4.367821 | DHX40 |
| 242569_at | 0.727277 | 1.655511 | 6.398927 | 4.540237 | 1.26E−05 | 0.000733 | 2.970642 | STAM2 |
| 1552310_at | 0.615811 | 1.532419 | 7.496994 | 4.492918 | 1.53E−05 | 0.000733 | 2.800826 | C15orf40 |
| 218010_x_at | −0.68332 | −1.60583 | 8.020818 | −4.40835 | 2.15E−05 | 0.000794 | 2.500492 | PPDPF |
| 242273_at | 0.730601 | 1.65933 | 6.563882 | 4.372567 | 2.48E−05 | 0.000794 | 2.374637 | LLPH |
| 225928_at | 0.806475 | 1.748933 | 6.926332 | 4.204792 | 4.81E−05 | 0.001283 | 1.794566 | VTI1B |
| 1554852_a_at | −0.67491 | −1.59649 | 4.441655 | −4.15845 | 5.76E−05 | 0.001283 | 1.637296 | CFAP92 |
| 205221_at | 1.131949 | 2.191545 | 5.521855 | 4.147254 | 6.01E−05 | 0.001283 | 1.599499 | HGD |
| 244033_at | 0.665526 | 1.586146 | 4.447717 | 3.92139 | 0.000141 | 0.002476 | 0.853438 | CEP128 |
| 204860_s_at | 0.828189 | 1.775456 | 4.624429 | 3.920459 | 0.000142 | 0.002476 | 0.850429 | NAIP |
| 223341_s_at | 0.659585 | 1.579628 | 9.159638 | 3.888355 | 0.00016 | 0.002479 | 0.747005 | SCOC |
| 228551_at | 0.736532 | 1.666166 | 4.783156 | 3.874901 | 0.000168 | 0.002479 | 0.703859 | DENND5B |
| 223721_s_at | 1.310652 | 2.480537 | 5.93828 | 3.798951 | 0.000222 | 0.003039 | 0.462478 | DNAJC12 |
| 238681_at | 0.751859 | 1.683961 | 5.734296 | 3.702733 | 0.000313 | 0.00401 | 0.16208 | GDPD1 |
| 202472_at | 0.654655 | 1.57424 | 4.913199 | 3.683657 | 0.000335 | 0.004023 | 0.103252 | MPI |
| 223315_at | 0.767466 | 1.702277 | 6.894435 | 3.664432 | 0.000359 | 0.004054 | 0.044206 | NTN4 |
| 230300_at | 0.689261 | 1.612458 | 5.899231 | 3.623149 | 0.000415 | 0.004273 | −0.08175 | PSMA5 |
| 226030_at | 0.659798 | 1.579861 | 6.255964 | 3.617967 | 0.000423 | 0.004273 | −0.09748 | ACADSB |
| 243552_at | 0.713432 | 1.6397 | 4.962934 | 3.598167 | 0.000453 | 0.004351 | −0.15741 | MBTD1 |
| 208126_s_at | 0.805346 | 1.747565 | 3.611484 | 3.530211 | 0.000574 | 0.005246 | −0.3611 | CYP2C18 |
| 244786_at | 0.678961 | 1.600986 | 5.033744 | 3.471049 | 0.000703 | 0.006134 | −0.53586 | SNHG10 |
| 227345_at | 0.616057 | 1.53268 | 6.051275 | 3.431211 | 0.000805 | 0.006477 | −0.65218 | TNFRSF10D |
| 209894_at | 0.711327 | 1.637309 | 7.083995 | 3.429323 | 0.00081 | 0.006477 | −0.65766 | LEPR |
| 233543_s_at | 0.741638 | 1.672073 | 4.923295 | 3.383206 | 0.000945 | 0.007261 | −0.79087 | ABRAXAS1 |
| 220235_s_at | 0.81248 | 1.756228 | 7.943655 | 3.337069 | 0.001102 | 0.008141 | −0.92265 | LRIF1 |
| 212224_at | 1.168851 | 2.248326 | 8.395865 | 3.290677 | 0.001285 | 0.009135 | −1.05364 | ALDH1A1 |
| 214261_s_at | 0.895806 | 1.860649 | 4.774624 | 3.272163 | 0.001365 | 0.00936 | −1.10548 | ADH6 |
| 1558279_a_at | 0.703098 | 1.627997 | 6.005751 | 3.250926 | 0.001463 | 0.009684 | −1.16465 | KDSR |
| 209355_s_at | 0.63223 | 1.549959 | 7.877788 | 3.223197 | 0.0016 | 0.01008 | −1.24143 | PLPP3 |
| 235609_at | 0.621524 | 1.538499 | 6.308716 | 3.206214 | 0.00169 | 0.01008 | −1.28817 | BRIP1 |
| 210397_at | 1.001097 | 2.001522 | 6.145694 | 3.206102 | 0.001691 | 0.01008 | −1.28848 | DEFB1 |
| 203116_s_at | 0.602827 | 1.518689 | 4.458316 | 3.198585 | 0.001732 | 0.01008 | −1.30911 | FECH |
| 202357_s_at | 0.735467 | 1.664936 | 8.974868 | 3.184143 | 0.001815 | 0.010247 | −1.34862 | CFB |
| 227892_at | 0.97473 | 1.965274 | 3.69031 | 3.169321 | 0.001903 | 0.010438 | −1.38901 | PRKAA2 |
| 240422_at | 0.710527 | 1.636402 | 4.930495 | 3.138274 | 0.0021 | 0.011201 | −1.4731 | FMO5 |
| 212094_at | 1.189922 | 2.281404 | 3.541903 | 3.101814 | 0.002356 | 0.012183 | −1.57096 | PEG10 |
| 219635_at | 0.630941 | 1.548575 | 5.788011 | 3.086277 | 0.002474 | 0.012183 | −1.61236 | ZNF606 |
| 1552627_a_at | 0.798687 | 1.739517 | 6.5662 | 3.077451 | 0.002543 | 0.012183 | −1.6358 | ARHGAP5 |
| 220773_s_at | 0.61834 | 1.535108 | 6.735038 | 3.071865 | 0.002588 | 0.012183 | −1.65061 | GPHN |
| 227722_at | 0.659571 | 1.579613 | 7.462596 | 3.055026 | 0.002728 | 0.012183 | −1.6951 | RPS23 |
| 1554930_a_at | 0.740259 | 1.670476 | 6.580271 | 3.052681 | 0.002748 | 0.012183 | −1.70128 | FUT8 |
| 230831_at | 0.878214 | 1.838099 | 5.587023 | 3.051135 | 0.002761 | 0.012183 | −1.70535 | FRMD5 |
| 226419_s_at | 0.660383 | 1.580502 | 7.978676 | 3.04753 | 0.002792 | 0.012183 | −1.71484 | SRSF1 |
| 206295_at | 0.761678 | 1.695462 | 7.228639 | 3.038672 | 0.00287 | 0.012244 | −1.73812 | IL18 |
| 227985_at | 0.771501 | 1.707045 | 5.287083 | 3.021449 | 0.003027 | 0.012424 | −1.7832 | LOC100506098 |
| 229883_at | −0.70673 | −1.6321 | 6.342097 | −3.01987 | 0.003041 | 0.012424 | −1.78731 | GRIN2D |
| 212398_at | 0.83425 | 1.78293 | 5.368229 | 2.993432 | 0.003299 | 0.012746 | −1.85607 | RDX |
| 204714_s_at | 0.986718 | 1.981671 | 4.821555 | 2.992152 | 0.003312 | 0.012746 | −1.85938 | F5 |
| 229435_at | 0.717894 | 1.64478 | 4.567109 | 2.991424 | 0.003319 | 0.012746 | −1.86127 | GLIS3 |
| 213695_at | 0.886371 | 1.848521 | 4.674797 | 2.977982 | 0.003458 | 0.01302 | −1.896 | PON3 |
| 223704_s_at | 0.774427 | 1.71051 | 3.583297 | 2.968038 | 0.003565 | 0.013163 | −1.92161 | DMRT2 |
| 230573_at | −0.65877 | −1.57873 | 6.269149 | −2.9284 | 0.00402 | 0.014308 | −2.02294 | SGK2 |
| 242093_at | 0.750684 | 1.682591 | 6.230241 | 2.927011 | 0.004037 | 0.014308 | −2.02648 | SYTL5 |
| 234082_at | 0.972595 | 1.962367 | 4.684465 | 2.921968 | 0.004099 | 0.014308 | −2.03928 | LOC100505874 |
| 213479_at | −1.42314 | −2.68169 | 5.148912 | −2.90938 | 0.004257 | 0.014595 | −2.07115 | NPTX2 |
| 227929_at | 0.64051 | 1.55888 | 4.244967 | 2.894712 | 0.004448 | 0.014777 | −2.10814 | LIN7A |
| 203210_s_at | 0.623846 | 1.540978 | 6.500834 | 2.888072 | 0.004537 | 0.014777 | −2.12484 | RFC5 |
| 202833_s_at | 1.016998 | 2.023703 | 10.8572 | 2.881402 | 0.004628 | 0.014777 | −2.14157 | SERPINA1 |
| 207753_at | 0.609513 | 1.525744 | 4.503888 | 2.880248 | 0.004644 | 0.014777 | −2.14446 | ZNF304 |
| 210815_s_at | 0.855228 | 1.809045 | 4.981442 | 2.876624 | 0.004695 | 0.014777 | −2.15354 | CALCRL |
| 216992_s_at | −0.84383 | −1.79481 | 5.479088 | −2.84601 | 0.005141 | 0.01592 | −2.2298 | GRM8 |
| 218025_s_at | 0.684331 | 1.606957 | 7.6457 | 2.836453 | 0.005288 | 0.016116 | −2.25346 | ECI2 |
| 224901_at | 0.681784 | 1.604123 | 6.007844 | 2.8135 | 0.005657 | 0.016971 | −2.31 | SCD5 |
| 1555564_a_at | 0.953617 | 1.936723 | 6.214389 | 2.796346 | 0.005948 | 0.017251 | −2.35199 | CFI |
| 238794_at | 0.73425 | 1.663532 | 6.213123 | 2.7941 | 0.005987 | 0.017251 | −2.35747 | SFR1 |
| 1554696_s_at | 0.633882 | 1.551735 | 6.762773 | 2.79221 | 0.00602 | 0.017251 | −2.36208 | TYMS |
| 210176_at | 0.715556 | 1.642116 | 4.409451 | 2.787001 | 0.006112 | 0.017257 | −2.37477 | TLR1 |
| 243042_at | 0.705994 | 1.631268 | 5.701297 | 2.769922 | 0.006423 | 0.017489 | −2.41623 | MIGA1 |
| 228365_at | 0.602641 | 1.518494 | 5.010849 | 2.765986 | 0.006496 | 0.017489 | −2.42576 | CPNE8 |
| 227134_at | 0.748539 | 1.680091 | 5.84204 | 2.76519 | 0.006511 | 0.017489 | −2.42768 | SYTL1 |
| 215933_s_at | 0.623056 | 1.540134 | 4.000618 | 2.76271 | 0.006558 | 0.017489 | −2.43368 | HHEX |
| 1559584_a_at | 0.66073 | 1.580882 | 3.752448 | 2.756597 | 0.006675 | 0.017556 | −2.44843 | C16orf54 |
| 227867_at | −0.69278 | −1.61639 | 8.794198 | −2.75065 | 0.006791 | 0.017619 | −2.46275 | TRABD2A |
| 238451_at | 0.698246 | 1.622531 | 5.16579 | 2.73174 | 0.00717 | 0.018187 | −2.50812 | MPP7 |
| 202340_x_at | −0.60141 | −1.5172 | 8.032047 | −2.72678 | 0.007273 | 0.018187 | −2.51998 | NR4A1 |
| 202376_at | 1.44288 | 2.718631 | 5.571625 | 2.725766 | 0.007294 | 0.018187 | −2.52239 | SERPINA3 |
| 226884_at | 0.698835 | 1.623194 | 3.242199 | 2.676268 | 0.008397 | 0.020669 | −2.6396 | LRRN1 |
| 229725_at | −1.22326 | −2.33474 | 5.839812 | −2.66791 | 0.008597 | 0.020895 | −2.6592 | ACSL6 |
| 206488_s_at | 0.838322 | 1.78797 | 5.340296 | 2.655071 | 0.008914 | 0.021257 | −2.68921 | CD36 |
| 217238_s_at | 1.201308 | 2.29948 | 5.727809 | 2.65185 | 0.008995 | 0.021257 | −2.69671 | ALDOB |
| 211478_s_at | 0.960313 | 1.945732 | 5.932936 | 2.648549 | 0.009078 | 0.021257 | −2.7044 | DPP4 |
| 209368_at | 0.635927 | 1.553936 | 5.750448 | 2.602641 | 0.010318 | 0.023548 | −2.8104 | EPHX2 |
| 221336_at | 0.641905 | 1.560389 | 3.253986 | 2.602412 | 0.010324 | 0.023548 | −2.81093 | ATOH1 |
| 222668_at | 0.607607 | 1.52373 | 6.014539 | 2.598905 | 0.010425 | 0.023548 | −2.81895 | KCTD15 |
| 1558028_x_at | 0.647428 | 1.566374 | 8.605164 | 2.586107 | 0.0108 | 0.024112 | −2.84817 | NORAD |
| 219313_at | 0.645675 | 1.564471 | 5.944988 | 2.564626 | 0.011457 | 0.025224 | −2.89692 | GRAMD1C |
| 205258_at | 0.644232 | 1.562907 | 4.890431 | 2.555172 | 0.011758 | 0.025224 | −2.91826 | INHBB |
| 228480_at | 0.605284 | 1.521279 | 4.632218 | 2.549169 | 0.011952 | 0.025224 | −2.93178 | VAPA |
| 235591_at | 0.665901 | 1.586559 | 4.106168 | 2.549169 | 0.011952 | 0.025224 | −2.93178 | SSTR1 |
| 224941_at | 0.658233 | 1.578149 | 5.298039 | 2.549071 | 0.011955 | 0.025224 | −2.932 | PAPPA |
| 204151_x_at | 0.644815 | 1.563539 | 8.059312 | 2.543195 | 0.012148 | 0.025353 | −2.9452 | AKR1C1 |
| 1552312_a_at | 0.651858 | 1.57119 | 5.645213 | 2.523616 | 0.012812 | 0.026315 | −2.98898 | MFAP3 |
| 226702_at | −0.62981 | −1.54736 | 8.376513 | −2.52156 | 0.012883 | 0.026315 | −2.99356 | CMPK2 |
| 204920_at | 0.8995 | 1.86542 | 3.887095 | 2.47125 | 0.014749 | 0.029769 | −3.10459 | CPS1 |
| 204924_at | 0.652835 | 1.572255 | 5.887157 | 2.467819 | 0.014885 | 0.029769 | −3.11209 | TLR2 |
| 229026_at | 0.714733 | 1.64118 | 9.161565 | 2.457816 | 0.015286 | 0.030258 | −3.13389 | LOC105379173 |
| 204041_at | 0.801603 | 1.743037 | 4.210569 | 2.44523 | 0.015806 | 0.030966 | −3.16122 | MAOB |
| 226907_at | −0.77532 | −1.71157 | 6.838824 | −2.43635 | 0.016181 | 0.031312 | −3.18043 | PPP1R14C |
| 226550_at | 0.601664 | 1.517466 | 4.985934 | 2.433393 | 0.016308 | 0.031312 | −3.1868 | SLC9A7 |
| 223620_at | 0.744981 | 1.675952 | 6.423797 | 2.40987 | 0.017349 | 0.03298 | −3.2373 | GPR34 |
| 204073_s_at | 0.793235 | 1.732956 | 5.809037 | 2.396177 | 0.017982 | 0.033848 | −3.2665 | MYRF |
| 229963_at | 0.669241 | 1.590237 | 5.625539 | 2.386504 | 0.018441 | 0.034375 | −3.28703 | BEX5 |
| 219747_at | 0.621245 | 1.538202 | 4.534081 | 2.382623 | 0.018628 | 0.03439 | −3.29524 | NDNF |
| 228564_at | 0.615186 | 1.531756 | 4.098846 | 2.348524 | 0.020346 | 0.036468 | −3.36691 | LINC01116 |
| 205302_at | 1.013319 | 2.01855 | 3.830896 | 2.345668 | 0.020496 | 0.036468 | −3.37287 | IGFBP1 |
| 205732_s_at | 0.64365 | 1.562276 | 5.787821 | 2.342094 | 0.020685 | 0.036468 | −3.38032 | NCOA2 |
| 218087_s_at | 0.623088 | 1.540168 | 8.251951 | 2.340176 | 0.020787 | 0.036468 | −3.38431 | SORBS1 |
| 238029_s_at | 0.637209 | 1.555317 | 5.305344 | 2.339262 | 0.020836 | 0.036468 | −3.38622 | SLC16A14 |
| 212705_x_at | −0.60719 | −1.52329 | 4.128512 | −2.3382 | 0.020893 | 0.036468 | −3.38843 | PNPLA2 |
| 209031_at | 0.642085 | 1.560583 | 5.763138 | 2.326159 | 0.021547 | 0.037055 | −3.41341 | CADM1 |
| 205220_at | 0.930993 | 1.906588 | 4.39853 | 2.321548 | 0.021802 | 0.037055 | −3.42294 | HCAR3 |
| 204959_at | 0.6954 | 1.619333 | 7.066125 | 2.321444 | 0.021808 | 0.037055 | −3.42316 | MNDA |
| 207500_at | 0.613084 | 1.529526 | 5.66595 | 2.313163 | 0.022273 | 0.037319 | −3.44024 | CASP5 |
| 211742_s_at | 0.62239 | 1.539423 | 7.070885 | 2.309279 | 0.022495 | 0.037319 | −3.44823 | EVI2B |
| 227949_at | 0.637689 | 1.555835 | 3.904181 | 2.308366 | 0.022547 | 0.037319 | −3.4501 | PHACTR3 |
| 238520_at | 0.648784 | 1.567846 | 5.17344 | 2.295927 | 0.02327 | 0.038186 | −3.47561 | TRERF1 |
| 227862_at | 0.688386 | 1.611479 | 6.414591 | 2.292328 | 0.023483 | 0.038209 | −3.48296 | TRNP1 |
| 205844_at | 0.871937 | 1.830118 | 6.446008 | 2.280023 | 0.024224 | 0.038612 | −3.50803 | VNN1 |
| 206404_at | 0.623987 | 1.541128 | 3.050925 | 2.279955 | 0.024228 | 0.038612 | −3.50817 | FGF9 |
| 204401_at | −0.62092 | −1.53786 | 8.171381 | −2.27822 | 0.024334 | 0.038612 | −3.51168 | KCNN4 |
| 211506_s_at | 0.862642 | 1.818365 | 9.293079 | 2.27224 | 0.024703 | 0.038632 | −3.52382 | CXCL8 |
| 1438_at | −0.72241 | −1.64994 | 6.999118 | −2.27151 | 0.024749 | 0.038632 | −3.52531 | EPHB3 |
| 206262_at | 0.971702 | 1.961152 | 7.096102 | 2.234624 | 0.027138 | 0.04202 | −3.59944 | ADH1C |
| 217320_at | 0.775961 | 1.71233 | 3.41296 | 2.225408 | 0.027766 | 0.042648 | −3.61779 | IGHV3-72 |
| 206326_at | −0.65956 | −1.5796 | 4.464591 | −2.21596 | 0.028422 | 0.04331 | −3.63653 | GRP |
| 206632_s_at | 0.653559 | 1.573044 | 7.077062 | 2.199945 | 0.029566 | 0.044698 | −3.66813 | APOBEC3B |
| 228821_at | 0.669741 | 1.590787 | 3.94935 | 2.173447 | 0.031547 | 0.04732 | −3.71994 | ST6GAL2 |
| 204273_at | 0.612807 | 1.529232 | 6.919864 | 2.148405 | 0.033523 | 0.049895 | −3.76838 | EDNRB |
| 225911_at | 0.604106 | 1.520037 | 8.37698 | 2.134582 | 0.034659 | 0.051189 | −3.79489 | NPNT |
| 215049_x_at | 0.695004 | 1.618889 | 6.193859 | 2.123032 | 0.035634 | 0.051823 | −3.81692 | CD163 |
| 207052_at | 0.703574 | 1.628534 | 3.202534 | 2.120565 | 0.035845 | 0.051823 | −3.82161 | HAVCR1 |
| 207259_at | 0.638692 | 1.556917 | 6.792053 | 2.119945 | 0.035898 | 0.051823 | −3.82279 | ANKRD40CL |
| 213680_at | 0.992885 | 1.990161 | 5.008728 | 2.105082 | 0.037196 | 0.053295 | −3.85094 | KRT6B |
| 205442_at | 0.659719 | 1.579775 | 5.915951 | 2.093713 | 0.038215 | 0.053991 | −3.87234 | MFAP3L |
| 234219_at | 0.692601 | 1.616195 | 5.300156 | 2.093402 | 0.038243 | 0.053991 | −3.87293 | LINC02535 |
| 226145_s_at | 0.699143 | 1.62354 | 4.467202 | 2.08392 | 0.039112 | 0.054814 | −3.8907 | FRAS1 |
| 206108_s_at | 0.614681 | 1.53122 | 6.179516 | 2.080547 | 0.039426 | 0.054853 | −3.897 | SRSF6 |
| 213094_at | 0.625444 | 1.542685 | 6.210305 | 2.060722 | 0.04131 | 0.057062 | −3.93385 | ADGRG6 |
| 205404_at | 0.642284 | 1.560798 | 5.080117 | 2.039616 | 0.043401 | 0.059522 | −3.97273 | HSD11B1 |
| 1555745_a_at | 1.115064 | 2.166045 | 8.824039 | 2.024833 | 0.044919 | 0.061166 | −3.99973 | LYZ |
| 206336_at | 0.819695 | 1.765033 | 6.069364 | 2.013338 | 0.04613 | 0.062373 | −4.0206 | CXCL6 |
| 222853_at | 0.691707 | 1.615193 | 5.812081 | 1.992988 | 0.048343 | 0.064908 | −4.05728 | FLRT3 |
| 202286_s_at | 0.974025 | 1.964313 | 9.136229 | 1.981001 | 0.049688 | 0.06625 | −4.07872 | TACSTD2 |
| 204724_s_at | −0.6213 | −1.53826 | 5.652595 | −1.96972 | 0.050983 | 0.067508 | −4.0988 | COL9A3 |
| 202086_at | −0.61436 | −1.53088 | 8.263696 | −1.96471 | 0.051567 | 0.067814 | −4.10768 | MX1 |
| 206884_s_at | 0.732723 | 1.661773 | 3.771019 | 1.956661 | 0.052517 | 0.068593 | −4.12189 | SCEL |
| 214146_s_at | 0.997936 | 1.997141 | 5.134884 | 1.947542 | 0.053611 | 0.069549 | −4.13793 | PPBP |
| 206207_at | 0.784308 | 1.722266 | 4.626893 | 1.930642 | 0.05569 | 0.071762 | −4.16747 | CLC |
| 237131_at | −0.6513 | −1.57058 | 4.200654 | −1.92718 | 0.056124 | 0.071838 | −4.17349 | RIIAD1 |
| 238127_at | −0.64239 | −1.56091 | 5.739427 | −1.9145 | 0.05774 | 0.072875 | −4.19547 | GAS6-AS1 |
| 205969_at | 0.71569 | 1.642268 | 4.006011 | 1.913478 | 0.057871 | 0.072875 | −4.19723 | AADAC |
| 1555203_s_at | 0.638523 | 1.556734 | 8.059392 | 1.911917 | 0.058073 | 0.072875 | −4.19993 | SLC44A4 |
| 206858_s_at | 0.857627 | 1.812056 | 4.34516 | 1.885957 | 0.061517 | 0.076697 | −4.24443 | HOXC6 |
| 229797_at | −0.61988 | −1.53674 | 4.822487 | −1.8375 | 0.068404 | 0.084733 | −4.32595 | MCOLN3 |
| 224367_at | 0.716699 | 1.643417 | 6.304159 | 1.802117 | 0.073829 | 0.090866 | −4.38421 | BEX2 |
| 229927_at | 0.672394 | 1.593715 | 4.799493 | 1.77648 | 0.077977 | 0.095361 | −4.42575 | LEMD1 |
| 216491_x_at | 0.778653 | 1.715528 | 7.398332 | 1.757528 | 0.081166 | 0.098353 | −4.4561 | IGHM |
| 206268_at | −0.99972 | −1.99961 | 8.577104 | −1.75588 | 0.081448 | 0.098353 | −4.45873 | LEFTY1 |
| 204439_at | −0.73977 | −1.66991 | 5.886474 | −1.74931 | 0.082582 | 0.098628 | −4.46916 | IFI44L |
| 234366_x_at | 0.664295 | 1.584794 | 5.572888 | 1.748607 | 0.082704 | 0.098628 | −4.47028 | IGLV@ |
| 202917_s_at | 0.745885 | 1.677002 | 8.734114 | 1.737806 | 0.084597 | 0.100263 | −4.48735 | S100A8 |
| 205267_at | 0.722668 | 1.650232 | 6.030163 | 1.734578 | 0.08517 | 0.100323 | −4.49243 | POU2AF1 |
| 214336_s_at | 0.605749 | 1.521769 | 5.959045 | 1.728606 | 0.086238 | 0.100962 | −4.50182 | COPA |

TABLE 16
Differential Expressed Genes FOLFIRI (All Stages CRC)

| | logFC | log2FC | AveExpr | t | P. Value | FDR | B | gene.symbols |
|---|---|---|---|---|---|---|---|---|
| 230774_at | 1.141207 | 2.205655 | 4.780488 | 4.184111 | 0.000105 | 0.002767 | 1.110903 | PTGR2 |
| 228766_at | −1.55246 | −2.93316 | 4.201463 | −3.93821 | 0.000236 | 0.002767 | 0.427515 | CD36 |
| 223700_at | 1.177574 | 2.261961 | 6.467592 | 3.936271 | 0.000238 | 0.002767 | 0.422205 | MND1 |
| 228396_at | −1.02611 | −2.03653 | 7.061739 | −3.88902 | 0.000277 | 0.002767 | 0.293243 | PRKG1 |
| 230130_at | −1.96003 | −3.89071 | 5.088919 | −3.72332 | 0.000469 | 0.003752 | −0.15252 | SLIT2 |
| 225328_at | −1.74276 | −3.34675 | 6.464618 | −3.58002 | 0.000734 | 0.00489 | −0.52948 | FBXO32 |
| 228176_at | −1.38538 | −2.6124 | 7.332569 | −3.49952 | 0.000939 | 0.0049 | −0.73751 | S1PR3 |
| 225442_at | −1.50212 | −2.83259 | 6.686699 | −3.48565 | 0.00098 | 0.0049 | −0.77308 | DDR2 |
| 227530_at | −1.59686 | −3.02485 | 6.016552 | −3.42845 | 0.001166 | 0.005181 | −0.91885 | AKAP12 |
| 223122_s_at | −3.12593 | −8.72969 | 7.235087 | −3.39133 | 0.001304 | 0.005215 | −1.01267 | SFRP2 |
| 242814_at | −1.40589 | −2.64981 | 5.681895 | −3.35775 | 0.001442 | 0.005243 | −1.09701 | SERPINB9 |
| 226084_at | −1.18757 | −2.27769 | 8.013852 | −3.24582 | 0.002009 | 0.006696 | −1.37437 | MAP1B |
| 227070_at | −1.18294 | −2.27039 | 7.613807 | −3.19054 | 0.002361 | 0.007265 | −1.50913 | GLT8D2 |
| 222877_at | −1.23735 | −2.35765 | 5.457327 | −3.13693 | 0.002757 | 0.007878 | −1.6384 | NRP2 |
| 236738_at | −1.4146 | −2.66586 | 4.102264 | −3.08179 | 0.00323 | 0.008612 | −1.76982 | C3orf80 |
| 235153_at | 1.104448 | 2.150166 | 4.783256 | 2.935904 | 0.004869 | 0.012173 | −2.10995 | RNF183 |
| 225450_at | −1.07884 | −2.11234 | 6.141223 | −2.67323 | 0.009903 | 0.021323 | −2.69281 | AMOTL1 |
| 227623_at | −1.01376 | −2.01917 | 5.587795 | −2.66562 | 0.010102 | 0.021323 | −2.7091 | CACNA2D1 |
| 225767_at | 1.379258 | 2.601346 | 9.861913 | 2.615829 | 0.011503 | 0.021323 | −2.81484 | RNA45SN5 |
| 223949_at | 1.231208 | 2.347635 | 5.057146 | 2.614312 | 0.011549 | 0.021323 | −2.81804 | TMPRSS3 |
| 225990_at | −1.03842 | −2.05398 | 5.51244 | −2.60349 | 0.011877 | 0.021323 | −2.84082 | BOC |
| 229947_at | −1.52835 | −2.88456 | 5.225503 | −2.59247 | 0.01222 | 0.021323 | −2.86392 | PI15 |
| 226834_at | −1.13069 | −2.18963 | 5.913305 | −2.59118 | 0.012261 | 0.021323 | −2.86664 | CLMP |
| 225381_at | −1.17487 | −2.25772 | 3.714421 | −2.5496 | 0.013642 | 0.022736 | −2.95315 | MIR100HG |
| 235244_at | 1.053849 | 2.076062 | 6.908893 | 2.494486 | 0.01569 | 0.025103 | −3.06619 | MIX23 |
| 225915_at | −1.30238 | −2.46636 | 5.062951 | −2.45958 | 0.017127 | 0.025459 | −3.1368 | CAB39L |
| 229927_at | −1.16767 | −2.24649 | 3.764306 | −2.45821 | 0.017185 | 0.025459 | −3.13955 | LEMD1 |
| 226930_at | −1.28716 | −2.44046 | 7.347232 | −2.3545 | 0.022195 | 0.031707 | −3.34463 | FNDC1 |
| 236179_at | −1.04054 | −2.057 | 5.334683 | −2.27989 | 0.026568 | 0.036646 | −3.4878 | CDH11 |
| 226777_at | −1.33871 | −2.52924 | 5.516604 | −2.18245 | 0.033423 | 0.043558 | −3.66916 | ADAM12 |
| 229479_at | −1.02511 | −2.03511 | 3.259201 | −2.17816 | 0.033758 | 0.043558 | −3.677 | LINC01614 |
| 229218_at | −1.34501 | −2.54033 | 8.164109 | −2.09244 | 0.04109 | 0.051362 | −3.83089 | COL1A2 |
| 228640_at | −1.24951 | −2.37761 | 6.203825 | −2.04197 | 0.046027 | 0.05579 | −3.91909 | PCDH7 |
| 227174_at | 1.61478 | 3.062648 | 6.724029 | 1.870964 | 0.066745 | 0.078524 | −4.20428 | WDR72 |
| 223395_at | −1.01571 | −2.0219 | 5.360935 | −1.83814 | 0.071518 | 0.081735 | −4.25656 | ABI3BP |
| 222722_at | −1.21585 | −2.32278 | 3.7502 | −1.82329 | 0.073768 | 0.081965 | −4.27994 | OGN |
| 227474_at | 1.150606 | 2.220071 | 7.128093 | 1.752684 | 0.085304 | 0.092196 | −4.38887 | PAX8-AS1 |
| 232458_at | −1.24643 | −2.37253 | 5.313176 | −1.73967 | 0.087586 | 0.092196 | −4.40853 | COL3A1 |
| 223597_at | −1.36349 | −2.57306 | 6.651864 | −1.31452 | 0.1942 | 0.199179 | −4.9777 | ITLN1 |
| 232252_at | 1.050325 | 2.070997 | 5.178527 | 1.124735 | 0.265651 | 0.265651 | −5.18413 | STYXL2 |

TABLE 17
Differentially Expressed Genes FOLFOX (CRC Early Stages)

| ID | logFC | log2FC | AveExpr | t | P. Value | FDR | B | gene.symbols |
|---|---|---|---|---|---|---|---|---|
| 226565_at | 1.088281 | 2.126205 | 6.084612 | 4.039576 | 0.000151 | 0.006835 | 0.242121 | KRT10-AS1 |
| 233543_s_at | 1.050402 | 2.071108 | 5.54949 | 3.858732 | 0.000276 | 0.006835 | −0.18706 | ABRAXAS1 |
| 212094_at | 2.595038 | 6.042051 | 4.054383 | 3.845019 | 0.000289 | 0.006835 | −0.2192 | PEG10 |
| 219360_s_at | −1.04675 | −2.06587 | 7.543799 | −3.43569 | 0.001065 | 0.017014 | −1.14969 | TRPM4 |
| 224367_at | 1.892958 | 3.713959 | 5.978751 | 3.3445 | 0.001409 | 0.017014 | −1.34867 | BEX2 |
| 210176_at | 1.119855 | 2.173251 | 4.496788 | 3.33785 | 0.001438 | 0.017014 | −1.36305 | TLR1 |
| 230666_at | −1.04321 | −2.0608 | 5.317301 | −3.21335 | 0.002091 | 0.020418 | −1.629 | HOXA11-AS |
| 213823_at | −1.27948 | −2.42752 | 5.337325 | −3.12956 | 0.002678 | 0.020418 | −1.80435 | HOXA11 |
| 227499_at | 1.133355 | 2.193683 | 4.772396 | 3.116956 | 0.002779 | 0.020418 | −1.83046 | FZD3 |
| 205286_at | 1.278176 | 2.425322 | 4.074213 | 3.099804 | 0.002921 | 0.020418 | −1.86588 | TFAP2C |
| 209541_at | 1.432765 | 2.699636 | 5.000199 | 3.072425 | 0.003163 | 0.020418 | −1.92215 | IGF1 |
| 223620_at | 1.180307 | 2.26625 | 7.0405 | 2.963294 | 0.004326 | 0.021916 | −2.14309 | GPR34 |
| 1438_at | −1.35578 | −2.55935 | 6.782028 | −2.95641 | 0.004411 | 0.021916 | −2.15685 | EPHB3 |
| 227892_at | 1.423835 | 2.682978 | 3.874688 | 2.904925 | 0.0051 | 0.021916 | −2.25901 | PRKAA2 |
| 210445_at | 1.195512 | 2.290262 | 6.971503 | 2.904754 | 0.005102 | 0.021916 | −2.25934 | FABP6 |
| 229007_at | 1.084188 | 2.120182 | 5.352112 | 2.888983 | 0.005333 | 0.021916 | −2.29038 | LOC283788 |
| 204688_at | 1.060237 | 2.085274 | 7.001361 | 2.885375 | 0.005387 | 0.021916 | −2.29747 | SGCE |
| 202340_x_at | −1.00664 | −2.00923 | 7.993128 | −2.86384 | 0.00572 | 0.021916 | −2.33963 | NR4A1 |
| 203705_s_at | 1.028076 | 2.039302 | 6.669883 | 2.854802 | 0.005865 | 0.021916 | −2.35725 | FZD7 |
| 204992_s_at | 1.079698 | 2.113594 | 9.556557 | 2.778283 | 0.007239 | 0.025551 | −2.50488 | PFN2 |
| 204783_at | 1.037683 | 2.052928 | 5.526292 | 2.762493 | 0.007557 | 0.025551 | −2.53499 | MLF1 |
| 239332_at | −1.1796 | −2.26515 | 8.140081 | −2.71621 | 0.008565 | 0.02648 | −2.62251 | LINC02086 |
| 1555989_at | 1.163463 | 2.239945 | 5.537998 | 2.715652 | 0.008578 | 0.02648 | −2.62355 | DAAM1 |
| 228195_at | 1.114697 | 2.165495 | 4.606433 | 2.654402 | 0.010103 | 0.027842 | −2.73768 | C2orf88 |
| 201117_s_at | 1.065358 | 2.092689 | 8.052116 | 2.641987 | 0.010441 | 0.027842 | −2.76058 | CPE |
| 229464_at | 1.042148 | 2.059292 | 5.796216 | 2.638703 | 0.010532 | 0.027842 | −2.76662 | MYEF2 |
| 208399_s_at | 1.022782 | 2.031833 | 3.973698 | 2.623158 | 0.010973 | 0.027842 | −2.79515 | EDN3 |
| 210397_at | 1.175606 | 2.258877 | 5.749686 | 2.604651 | 0.01152 | 0.027842 | −2.82895 | DEFB1 |
| 210675_s_at | 1.361393 | 2.569332 | 5.298266 | 2.594121 | 0.011842 | 0.027842 | −2.8481 | PTPRR |
| 213147_at | −1.00483 | −2.00671 | 7.326362 | −2.58484 | 0.012133 | 0.027842 | −2.86494 | HOXA10 |
| 223721_s_at | 1.478405 | 2.786405 | 5.788999 | 2.584085 | 0.012156 | 0.027842 | −2.8663 | DNAJC12 |
| 201849_at | 1.303607 | 2.468452 | 8.434126 | 2.56523 | 0.012768 | 0.028329 | −2.90034 | BNIP3 |
| 227949_at | 1.058636 | 2.082962 | 4.062751 | 2.535789 | 0.013779 | 0.028728 | −2.95312 | PHACTR3 |
| 224396_s_at | 1.344757 | 2.539874 | 6.490304 | 2.525768 | 0.014139 | 0.028728 | −2.97098 | ASPN |
| 205442_at | 1.231509 | 2.348125 | 6.335 | 2.525141 | 0.014162 | 0.028728 | −2.9721 | MFAP3L |
| 230831_at | 1.002072 | 2.002874 | 5.72492 | 2.502164 | 0.015021 | 0.029625 | −3.01283 | FRMD5 |
| 226884_at | 1.081525 | 2.116272 | 3.602112 | 2.477448 | 0.015998 | 0.030699 | −3.05632 | LRRN1 |
| 218963_s_at | 1.7364 | 3.332028 | 8.490825 | 2.416843 | 0.01864 | 0.034205 | −3.16155 | KRT23 |
| 214146_s_at | 1.749569 | 3.362581 | 4.849617 | 2.410226 | 0.018951 | 0.034205 | −3.17292 | PPBP |
| 226145_s_at | 1.04074 | 2.057282 | 4.827291 | 2.403538 | 0.01927 | 0.034205 | −3.18438 | FRAS1 |
| 212224_at | 1.18154 | 2.268188 | 8.313838 | 2.366963 | 0.021101 | 0.036005 | −3.24662 | ALDH1A1 |
| 216255_s_at | −1.11866 | −2.17146 | 4.439653 | −2.36319 | 0.021299 | 0.036005 | −3.253 | GRM8 |
| 206755_at | −1.02507 | −2.03505 | 6.454397 | −2.3423 | 0.022422 | 0.037023 | −3.28816 | CYP2B6 |
| 206488_s_at | 1.115268 | 2.166353 | 4.907617 | 2.264732 | 0.027069 | 0.042817 | −3.41653 | CD36 |
| 1559633_a_at | −1.17329 | −2.25525 | 5.125933 | −2.25669 | 0.027596 | 0.042817 | −3.42964 | CHRM3 |
| 206134_at | 1.102321 | 2.146998 | 10.01319 | 2.254508 | 0.027741 | 0.042817 | −3.43319 | ADAMDEC1 |
| 205295_at | 1.439059 | 2.711439 | 5.245949 | 2.240806 | 0.028664 | 0.043302 | −3.45543 | CKMT2 |
| 205979_at | 1.078713 | 2.11215 | 3.638461 | 2.187079 | 0.032554 | 0.048152 | −3.54153 | SCGB2A1 |

TABLE 18
Differentially Expressed Genes FOLFIRI (CRC Early Stage)

| ID | logFC | log2FC | AveExpr | ID | P. Value | FDR | B | gene.symbols |
|---|---|---|---|---|---|---|---|---|
| 222722_at | −1.29887 | −2.46037 | 4.30054 | −2.08716 | 0.040799 | 0.045506 | −3.84684 | OGN |
| 222853_at | 1.129139 | 2.187282 | 5.455819 | 2.499643 | 0.014966 | 0.027125 | −3.38637 | FLRT3 |
| 223122_s_at | −2.07948 | −4.22654 | 7.466847 | −2.45395 | 0.016818 | 0.02869 | −3.4405 | SFRP2 |
| 223395_at | −1.05623 | −2.07949 | 6.204503 | −2.11773 | 0.038028 | 0.044112 | −3.81497 | ABI3BP |
| 224396_s_at | −1.38304 | −2.60817 | 5.077538 | −2.52263 | 0.014105 | 0.027125 | −3.35886 | ASPN |
| 225381_at | −1.56844 | −2.96584 | 4.819194 | −3.36523 | 0.001287 | 0.007348 | −2.23595 | MIR100HG |
| 225442_at | −1.11653 | −2.16825 | 7.548933 | −3.1118 | 0.002762 | 0.008901 | −2.59511 | DDR2 |
| 225681_at | −1.12393 | −2.1794 | 11.1113 | −3.06562 | 0.003163 | 0.009171 | −2.65875 | CTHRC1 |
| 226237_at | −1.20971 | −2.31292 | 9.529254 | −2.96729 | 0.004201 | 0.010152 | −2.79223 | COL8A1 |
| 226777_at | −1.26833 | −2.40883 | 6.084634 | −2.42616 | 0.018043 | 0.02907 | −3.47306 | ADAM12 |
| 226930_at | −1.11238 | −2.16202 | 7.841961 | −2.37182 | 0.020672 | 0.031373 | −3.5359 | FNDC1 |
| 227070_at | −1.06589 | −2.09346 | 8.226225 | −3.2465 | 0.001849 | 0.007348 | −2.40626 | GLT8D2 |
| 227530_at | −1.39799 | −2.63534 | 6.707041 | −3.25499 | 0.001802 | 0.007348 | −2.3942 | AKAP12 |
| 227758_at | −1.00055 | −2.00076 | 6.180238 | −2.92686 | 0.004714 | 0.010515 | −2.84631 | RERG |
| 228080_at | −1.00536 | −2.00744 | 5.732275 | −2.98224 | 0.004025 | 0.010152 | −2.77212 | LAYN |
| 228640_at | −1.08728 | −2.12473 | 7.182428 | −2.25595 | 0.027445 | 0.034605 | −3.66625 | PCDH7 |
| 228827_at | −1.19622 | −2.29139 | 5.252839 | −3.30956 | 0.001527 | 0.007348 | −2.31623 | RUNX1T1 |
| 229218_at | −1.18058 | −2.26668 | 9.201711 | −2.25891 | 0.02725 | 0.034605 | −3.66297 | COL1A2 |
| 229554_at | −1.09212 | −2.13186 | 8.189921 | −3.24691 | 0.001847 | 0.007348 | −2.40567 | LUM |
| 229667_s_at | 1.098708 | 2.141628 | 5.635789 | 2.202036 | 0.031217 | 0.03772 | −3.72515 | HOXB8 |
| 229802_at | −1.40129 | −2.64137 | 6.91947 | −3.38329 | 0.001217 | 0.007348 | −2.20973 | CCN4 |
| 231240_at | −1.12827 | −2.18597 | 5.673999 | −2.62588 | 0.010765 | 0.022299 | −3.23304 | DIO2 |
| 232252_at | −1.72327 | −3.30183 | 4.979202 | −2.26454 | 0.026883 | 0.034605 | −3.65676 | STYXL2 |
| 233371_at | 1.035887 | 2.050374 | 3.267701 | 3.363751 | 0.001293 | 0.007348 | −2.23809 | ABCC13 |
| 236044_at | −1.23142 | −2.34798 | 4.87359 | −2.35342 | 0.021636 | 0.031373 | −3.55693 | PLPP4 |
| 236179_at | −1.32523 | −2.50573 | 6.208704 | −3.21591 | 0.002027 | 0.007348 | −2.44956 | CDH11 |

TABLE 19
Differentially expressed Genes FOLFOX (CRC Metastatic Stages)

| | logFC | log2FC | AveExpr | t | P. Value | FDR | B | gene.symbols |
|---|---|---|---|---|---|---|---|---|
| 225915_at | −1.93678 | −3.82849 | 5.385789 | −3.70457 | 0.000502 | 0.010905 | −0.47979 | CAB39L |
| 202286_s_at | −1.87193 | −3.66021 | 8.66868 | −2.35268 | 0.022335 | 0.035413 | −3.27244 | TACSTD2 |
| 213350_at | −1.77812 | −3.42978 | 8.19465 | −2.69482 | 0.009382 | 0.023329 | −2.64608 | RPS11 |
| 213826_s_at | −1.70454 | −3.25924 | 6.53503 | −3.57873 | 0.000742 | 0.011287 | −0.77134 | H3-3A |
| 200908_s_at | −1.59436 | −3.01962 | 5.027551 | −3.26065 | 0.001934 | 0.013832 | −1.48338 | RPLP2 |
| 227919_at | −1.5171 | −2.86215 | 7.565358 | −2.16032 | 0.035231 | 0.04927 | −3.59564 | UCA1 |
| 204364_s_at | −1.42199 | −2.67955 | 5.350338 | −2.72778 | 0.008598 | 0.022847 | −2.58248 | REEP1 |
| 214395_x_at | −1.39106 | −2.62272 | 4.797131 | −4.64127 | 2.27E−05 | 0.004222 | 1.824311 | EEF1D |
| 204537_s_at | −1.38368 | −2.60932 | 7.039462 | −3.28591 | 0.001795 | 0.013832 | −1.42822 | GABRE |
| 212044_s_at | −1.38324 | −2.60854 | 8.595366 | −3.09619 | 0.003114 | 0.014513 | −1.83606 | RPL27A |
| 213642_at | −1.36328 | −2.57269 | 9.388377 | −3.446 | 0.001113 | 0.012605 | −1.07297 | RPL27 |
| 210393_at | −1.3584 | −2.56401 | 5.130629 | −2.41508 | 0.019168 | 0.033123 | −3.16297 | LGR5 |
| 214001_x_at | −1.29728 | −2.45765 | 7.454339 | −3.91694 | 0.000255 | 0.010905 | 0.023405 | RPS10 |
| 229215_at | −1.25349 | −2.38418 | 10.29626 | −2.35924 | 0.021981 | 0.035413 | −3.26103 | ASCL2 |
| 216971_s_at | −1.23622 | −2.3558 | 7.109085 | −3.17575 | 0.002477 | 0.014433 | −1.66684 | PLEC |
| 229420_at | −1.2009 | −2.29883 | 8.022769 | −3.7297 | 0.000464 | 0.010905 | −0.42096 | LINC02067 |
| 227556_at | −1.1852 | −2.27394 | 12.03257 | −3.13127 | 0.002816 | 0.014433 | −1.76177 | NME7 |
| 229228_at | −1.12256 | −2.17733 | 3.909144 | −3.56518 | 0.000773 | 0.011287 | −0.8024 | CREB5 |
| 222245_s_at | −1.12052 | −2.17426 | 5.814249 | −2.96462 | 0.004513 | 0.016788 | −2.10985 | FER1L4 |
| 1557207_s_at | −1.11504 | −2.166 | 5.111603 | −3.1401 | 0.002745 | 0.014433 | −1.743 | B3GAT1-DT |
| 238214_at | −1.08813 | −2.12599 | 4.224392 | −2.51873 | 0.014791 | 0.03061 | −2.97635 | LRRC69 |
| 219189_at | −1.08124 | −2.11585 | 6.356228 | −3.13357 | 0.002797 | 0.014433 | −1.75689 | FBXL6 |
| 239332_at | −1.06565 | −2.09312 | 8.14979 | −2.44534 | 0.017783 | 0.032114 | −3.1091 | LINC02086 |
| 238853_at | −1.05108 | −2.07208 | 5.572636 | −2.67833 | 0.009798 | 0.02398 | −2.67768 | RAB3IP |
| 227475_at | −1.04704 | −2.06629 | 10.28181 | −2.36001 | 0.02194 | 0.035413 | −3.25969 | FOXQ1 |
| 218641_at | −1.03168 | −2.0444 | 7.310585 | −3.48294 | 0.000995 | 0.012335 | −0.98966 | ZFTA |
| 213348_at | −1.02757 | −2.03858 | 6.125819 | −2.66052 | 0.010267 | 0.0248 | −2.71168 | CDKN1C |
| 228144_at | −1.02566 | −2.03589 | 3.743796 | −2.40999 | 0.019411 | 0.033123 | −3.17198 | ZNF300 |
| 221173_at | −1.0243 | −2.03397 | 7.547596 | −2.46721 | 0.016839 | 0.03196 | −3.06985 | USH1C |
| 209433_s_at | −1.01289 | −2.01794 | 7.35956 | −2.98735 | 0.004235 | 0.016488 | −2.06308 | PPAT |
| 201367_s_at | −1.00968 | −2.01347 | 8.70187 | −2.2663 | 0.027488 | 0.040577 | −3.42026 | ZFP36L2 |
| 238755_at | −1.00758 | −2.01053 | 4.723981 | −2.29866 | 0.025445 | 0.039114 | −3.36539 | RASSF10 |
| 206558_at | −1.00603 | −2.00837 | 4.858833 | −2.83632 | 0.006424 | 0.01993 | −2.36922 | SIM2 |
| 211766_s_at | 1.004002 | 2.005556 | 3.89409 | 2.194362 | 0.032557 | 0.046226 | −3.54004 | PNLIPRP2 |
| 205141_at | 1.014942 | 2.020822 | 7.900493 | 2.818026 | 0.00675 | 0.01993 | −2.40556 | ANG |
| 205488_at | 1.020593 | 2.028753 | 4.724986 | 2.484927 | 0.016107 | 0.031208 | −3.03786 | GZMA |
| 206172_at | 1.022887 | 2.031981 | 3.351445 | 2.384209 | 0.020681 | 0.034969 | −3.2174 | IL13RA2 |
| 228376_at | 1.023417 | 2.032728 | 5.681619 | 4.108065 | 0.000137 | 0.010905 | 0.487054 | GGTA1 |
| 211742_s_at | 1.027445 | 2.038411 | 6.699449 | 2.55006 | 0.013659 | 0.029541 | −2.91879 | EVI2B |
| 206461_x_at | 1.030067 | 2.04212 | 9.906528 | 2.896982 | 0.005442 | 0.018456 | −2.24754 | MT1H |
| 203939_at | 1.034287 | 2.048101 | 8.83074 | 2.31573 | 0.024423 | 0.037856 | −3.3362 | NT5E |
| 224480_s_at | 1.03482 | 2.048858 | 5.524019 | 2.820273 | 0.00671 | 0.01993 | −2.40111 | GPAT3 |
| 204519_s_at | 1.036166 | 2.05077 | 5.945661 | 2.512906 | 0.015011 | 0.030681 | −2.98699 | PLLP |
| 202388_at | 1.040718 | 2.057251 | 9.268773 | 2.728807 | 0.008575 | 0.022847 | −2.58048 | RGS2 |
| 206637_at | 1.041925 | 2.058973 | 4.155026 | 2.361636 | 0.021853 | 0.035413 | −3.25685 | P2RY14 |
| 204007_at | 1.04754 | 2.067003 | 5.816403 | 2.266454 | 0.027478 | 0.040577 | −3.42 | FCGR3B |
| 223059_s_at | 1.05635 | 2.079663 | 8.342245 | 3.282231 | 0.001815 | 0.013832 | −1.43627 | FAM107B |
| 220330_s_at | 1.057349 | 2.081103 | 6.873295 | 2.518184 | 0.014811 | 0.03061 | −2.97735 | SAMSN1 |
| 226811_at | 1.067459 | 2.095738 | 7.61628 | 2.598229 | 0.01207 | 0.027717 | −2.82926 | TENT5C |
| 217165_x_at | 1.074009 | 2.105275 | 8.91981 | 3.173337 | 0.002494 | 0.014433 | −1.67201 | MT1F |
| 209398_at | 1.075262 | 2.107105 | 7.134653 | 3.188319 | 0.002388 | 0.014433 | −1.63987 | H1-2 |
| 218211_s_at | 1.080438 | 2.114678 | 8.056233 | 2.276922 | 0.026802 | 0.040203 | −3.40232 | MLPH |
| 229390_at | 1.088415 | 2.126403 | 6.902399 | 2.431472 | 0.018406 | 0.032919 | −3.13385 | CALHM6 |
| 215440_s_at | 1.09023 | 2.129079 | 6.500383 | 2.979779 | 0.004326 | 0.016488 | −2.07869 | BEX4 |
| 205290_s_at | 1.091853 | 2.131476 | 5.851932 | 2.503668 | 0.015365 | 0.031064 | −3.00384 | BMP2 |
| 206710_s_at | 1.094586 | 2.135518 | 5.056989 | 2.693816 | 0.009407 | 0.023329 | −2.64801 | EPB41L3 |
| 221530_s_at | 1.095963 | 2.137558 | 7.018953 | 2.578595 | 0.012696 | 0.027854 | −2.8659 | BHLHE41 |
| 204719_at | 1.096865 | 2.138894 | 4.583046 | 2.592737 | 0.012243 | 0.02777 | −2.83953 | ABCA8 |
| 210174_at | 1.103911 | 2.149366 | 5.058827 | 2.712303 | 0.008959 | 0.023143 | −2.61241 | NR5A2 |
| 207222_at | 1.121317 | 2.175455 | 6.936773 | 2.878304 | 0.005729 | 0.019027 | −2.28519 | PLA2G10 |
| 228969_at | 1.137361 | 2.199783 | 8.903296 | 2.577591 | 0.012729 | 0.027854 | −2.86777 | AGR2 |
| 202953_at | 1.141374 | 2.20591 | 8.563938 | 3.036274 | 0.003691 | 0.015256 | −1.96168 | C1QB |
| 232428_at | 1.148126 | 2.216259 | 4.512461 | 3.434467 | 0.001152 | 0.012605 | −1.0989 | MOGAT2 |
| 233819_s_at | 1.151651 | 2.22168 | 5.192768 | 2.615026 | 0.011558 | 0.026871 | −2.79775 | LTN1 |
| 228004_at | 1.153001 | 2.22376 | 4.07436 | 2.292382 | 0.02583 | 0.039381 | −3.37608 | LINC00261 |
| 204378_at | 1.165992 | 2.243875 | 6.097485 | 3.140886 | 0.002739 | 0.014433 | −1.74132 | BCAS1 |
| 206067_s_at | 1.171547 | 2.252531 | 4.387724 | 2.779643 | 0.007486 | 0.021423 | −2.4813 | WT1 |
| 223484_at | 1.172411 | 2.253881 | 11.372 | 2.446565 | 0.017729 | 0.032114 | −3.10691 | C15orf48 |
| 1552870_s_at | 1.183056 | 2.270573 | 3.71582 | 2.863511 | 0.005965 | 0.019465 | −2.31489 | AXDND1 |
| 206932_at | 1.188123 | 2.278561 | 5.297944 | 2.75395 | 0.00802 | 0.022263 | −2.53159 | CH25H |
| 229831_at | 1.195669 | 2.29051 | 3.431894 | 2.714752 | 0.008901 | 0.023143 | −2.60768 | CNTN3 |
| 200795_at | 1.199028 | 2.295849 | 9.270505 | 2.412367 | 0.019297 | 0.033123 | −3.16778 | SPARCL1 |
| 238029_s_at | 1.199577 | 2.296723 | 4.958589 | 3.073287 | 0.003323 | 0.014718 | −1.88426 | SLC16A14 |
| 203021_at | 1.222717 | 2.333858 | 9.292365 | 2.41885 | 0.018991 | 0.033123 | −3.15629 | SLPI |
| 222838_at | 1.235662 | 2.354893 | 4.839713 | 2.741432 | 0.008292 | 0.022681 | −2.55597 | SLAMF7 |
| 228232_s_at | 1.236454 | 2.356188 | 6.487731 | 2.197577 | 0.032314 | 0.046226 | −3.53475 | VSIG2 |
| 37512_at | 1.256573 | 2.389275 | 5.262473 | 2.802283 | 0.007044 | 0.020471 | −2.43671 | HSD17B6 |
| 215777_at | 1.26557 | 2.404221 | 3.405319 | 2.412267 | 0.019302 | 0.033123 | −3.16795 | IGLV4-60 |
| 207080_s_at | 1.272574 | 2.415923 | 3.219673 | 3.117991 | 0.002925 | 0.014433 | −1.78995 | PYY |
| 204273_at | 1.283687 | 2.434603 | 5.937427 | 3.095348 | 0.003121 | 0.014513 | −1.83783 | EDNRB |
| 204036_at | 1.28702 | 2.440235 | 4.483219 | 2.917287 | 0.005146 | 0.018456 | −2.20642 | LPAR1 |
| 208450_at | 1.296987 | 2.457151 | 5.450936 | 2.356719 | 0.022116 | 0.035413 | −3.26541 | LGALS2 |
| 205464_at | 1.304797 | 2.47049 | 3.704048 | 2.52972 | 0.014384 | 0.030403 | −2.95622 | SCNN1B |
| 203474_at | 1.315786 | 2.48938 | 8.159262 | 3.719044 | 0.000479 | 0.010905 | −0.44593 | IQGAP2 |
| 209541_at | 1.321335 | 2.498972 | 4.477681 | 3.486691 | 0.000983 | 0.012335 | −0.98117 | IGF1 |
| 228195_at | 1.376033 | 2.595537 | 4.581294 | 3.225731 | 0.002142 | 0.014227 | −1.55919 | C2orf88 |
| 203889_at | 1.389227 | 2.619383 | 6.350394 | 3.635145 | 0.000623 | 0.011287 | −0.64127 | SCG5 |
| 204697_s_at | 1.393585 | 2.627308 | 4.855586 | 2.241036 | 0.029182 | 0.042405 | −3.46268 | CHGA |
| 221024_s_at | 1.404454 | 2.647175 | 7.381216 | 3.688393 | 0.000528 | 0.010905 | −0.51756 | SLC2A10 |
| 223395_at | 1.409989 | 2.657352 | 5.58913 | 3.295017 | 0.001747 | 0.013832 | −1.40828 | ABI3BP |
| 207522_s_at | 1.430589 | 2.695567 | 6.771684 | 2.822855 | 0.006663 | 0.01993 | −2.39598 | ATP2A3 |
| 228058_at | 1.430959 | 2.69626 | 7.376125 | 2.328561 | 0.023679 | 0.037011 | −3.31414 | ZG16B |
| 217238_s_at | 1.444006 | 2.720753 | 6.021886 | 2.194655 | 0.032535 | 0.046226 | −3.53956 | ALDOB |
| 211798_x_at | 1.497584 | 2.823695 | 6.777277 | 2.445624 | 0.017771 | 0.032114 | −3.10859 | IGLI3 |
| 214375_at | 1.514174 | 2.856353 | 6.000353 | 3.280286 | 0.001825 | 0.013832 | −1.44053 | PPFIBP1 |
| 1555963_x_at | 1.547407 | 2.922914 | 6.191535 | 2.766733 | 0.00775 | 0.021841 | −2.50661 | B3GNT7 |
| 234366_x_at | 1.589031 | 3.008473 | 4.799652 | 2.490767 | 0.015873 | 0.031198 | −3.02728 | IGLV@ |
| 214777_at | 1.607426 | 3.047077 | 7.314544 | 2.156577 | 0.035536 | 0.049326 | −3.60171 | IGKV4-1 |
| 208383_s_at | 1.643436 | 3.124091 | 8.863537 | 2.357083 | 0.022097 | 0.035413 | −3.26478 | PCK1 |
| 217546_at | 1.712409 | 3.277075 | 4.287439 | 3.271775 | 0.001871 | 0.013832 | −1.45912 | MT1M |
| 211644_x_at | 1.718553 | 3.291062 | 8.430474 | 2.277475 | 0.026766 | 0.040203 | −3.40138 | IGKC |
| 206641_at | 1.728244 | 3.313244 | 3.625308 | 3.115205 | 0.002949 | 0.014433 | −1.79585 | TNFRSF17 |
| 215214_at | 1.73195 | 3.321764 | 7.704729 | 2.900214 | 0.005394 | 0.018456 | −2.24101 | IGLV3-25 |
| 212592_at | 1.743653 | 3.348821 | 9.701978 | 2.365498 | 0.021649 | 0.035413 | −3.25013 | JCHAIN |
| 214598_at | 1.751373 | 3.366789 | 3.999127 | 2.624088 | 0.011289 | 0.02658 | −2.78069 | CLDN8 |
| 238750_at | 1.767861 | 3.405486 | 5.543485 | 3.558749 | 0.000789 | 0.011287 | −0.81713 | CCL28 |
| 219669_at | 1.77546 | 3.423472 | 4.311937 | 2.578015 | 0.012715 | 0.027854 | −2.86698 | CD177 |
| 216560_x_at | 1.780083 | 3.434459 | 6.087735 | 2.453971 | 0.017405 | 0.032114 | −3.09364 | IGLV3-10 |
| 229070_at | 1.805043 | 3.494397 | 5.990343 | 2.895958 | 0.005458 | 0.018456 | −2.24961 | ADTRP |
| 217148_x_at | 1.818748 | 3.52775 | 8.120324 | 2.448568 | 0.017641 | 0.032114 | −3.10332 | IGLV2-14 |
| 223969_s_at | 1.859533 | 3.628902 | 4.156334 | 3.007248 | 0.004006 | 0.016196 | −2.02198 | RETNLB |
| 209301_at | 1.867132 | 3.648066 | 7.977519 | 3.06471 | 0.003405 | 0.014731 | −1.90225 | CA2 |
| 204818_at | 1.869116 | 3.653088 | 6.065341 | 3.077975 | 0.003279 | 0.014718 | −1.87441 | HSD17B2 |
| 205554_s_at | 1.904475 | 3.743727 | 4.54742 | 3.798833 | 0.000373 | 0.010905 | −0.2581 | DNASE1L3 |
| 206134_at | 1.909393 | 3.756509 | 8.103353 | 3.721981 | 0.000475 | 0.010905 | −0.43905 | ADAMDEC1 |
| 209613_s_at | 1.92122 | 3.787431 | 5.29524 | 2.697882 | 0.009307 | 0.023329 | −2.64019 | ADH1B |
| 206561_s_at | 1.922953 | 3.791984 | 5.839293 | 3.047205 | 0.003579 | 0.015128 | −1.93888 | AKR1B10 |
| 210133_at | 1.924906 | 3.797121 | 4.717043 | 2.901925 | 0.005369 | 0.018456 | −2.23755 | CCL11 |
| 206664_at | 1.925085 | 3.797593 | 4.619699 | 2.491755 | 0.015833 | 0.031198 | −3.02549 | SI |
| 205267_at | 1.954726 | 3.876424 | 5.143796 | 3.370606 | 0.001396 | 0.012981 | −1.24149 | POU2AF1 |
| 202917_s_at | 1.995086 | 3.9864 | 8.336591 | 3.152096 | 0.002652 | 0.014433 | −1.71743 | S100A8 |
| 227725_at | 2.023339 | 4.065236 | 8.60708 | 2.825369 | 0.006618 | 0.01993 | −2.39099 | ST6GALNAC1 |
| 220834_at | 2.144073 | 4.420082 | 5.111113 | 2.253697 | 0.028322 | 0.041479 | −3.44147 | MS4A12 |
| 203240_at | 2.162469 | 4.476804 | 7.753294 | 2.469629 | 0.016737 | 0.03196 | −3.06549 | FCGBP |
| 206422_at | 2.169533 | 4.498777 | 3.614376 | 3.414361 | 0.001224 | 0.012649 | −1.14395 | GCG |
| 205950_s_at | 2.200936 | 4.597777 | 6.103432 | 2.170749 | 0.034392 | 0.048461 | −3.57868 | CA1 |
| 214974_x_at | 2.238811 | 4.720078 | 6.298461 | 2.649322 | 0.010572 | 0.025209 | −2.73296 | CXCL5 |
| 213432_at | 2.259593 | 4.788563 | 6.920979 | 2.854535 | 0.006113 | 0.019604 | −2.33286 | MUC5B |
| 210107_at | 2.317444 | 4.984484 | 5.7624 | 2.350268 | 0.022466 | 0.035413 | −3.27662 | CLCA1 |
| 242601_at | 2.447905 | 5.456231 | 4.709445 | 3.376196 | 0.001373 | 0.012981 | −1.22907 | HEPACAM2 |
| 214142_at | 2.650024 | 6.276779 | 6.221971 | 2.489227 | 0.015934 | 0.031198 | −3.03007 | ZG16 |
| 223597_at | 2.669146 | 6.360526 | 5.27534 | 2.978323 | 0.004344 | 0.016488 | −2.08168 | ITLN1 |
| 223447_at | 2.862181 | 7.271137 | 7.85174 | 2.535962 | 0.014158 | 0.030269 | −2.94475 | REG4 |
| 204673_at | 3.396101 | 10.52757 | 7.386924 | 3.238496 | 0.002063 | 0.014214 | −1.53153 | MUC2 |

TABLE 20
Differentially expressed Genes FOLFIRI (CRC Metastatic Stages)

| | logFC | log2FC | AveExpr | t | P. Value | FDR | B | gene.symbols |
|---|---|---|---|---|---|---|---|---|
| 229947_at | −2.4666 | −5.52738 | 5.376081 | −3.28229 | 0.002871 | 0.065671 | −3.46746 | PI15 |
| 228640_at | −1.87656 | −3.67197 | 5.421119 | −2.1734 | 0.038792 | 0.092503 | −4.11162 | PCDH7 |
| 230130_at | −1.82423 | −3.54117 | 4.947842 | −2.32362 | 0.028006 | 0.089153 | −4.02933 | SLIT2 |
| 244056_at | −1.75521 | −3.37576 | 5.832426 | −2.3651 | 0.025552 | 0.089153 | −4.00619 | SFTA2 |
| 225328_at | −1.70642 | −3.26349 | 6.371084 | −2.60289 | 0.0149 | 0.089153 | −3.87063 | FBXO32 |
| 226930_at | −1.70103 | −3.25133 | 7.435464 | −2.12764 | 0.042751 | 0.094093 | −4.13617 | FNDC1 |
| 238853_at | −1.60685 | −3.04587 | 3.961988 | −3.20779 | 0.00346 | 0.065671 | −3.51216 | RAB3IP |
| 227812_at | −1.59871 | −3.02873 | 3.800377 | −2.82702 | 0.008791 | 0.089153 | −3.73937 | TNFRSF19 |
| 232099_at | −1.57968 | −2.98904 | 4.207487 | −3.0609 | 0.004982 | 0.077221 | −3.60017 | PCDHB16 |
| 225442_at | −1.50604 | −2.8403 | 6.38793 | −2.95975 | 0.006382 | 0.084784 | −3.66057 | DDR2 |
| 232458_at | −1.45291 | −2.7376 | 3.898862 | −2.35442 | 0.026164 | 0.089153 | −4.01216 | COL3A1 |
| 236179_at | −1.38753 | −2.61631 | 5.075089 | −2.11463 | 0.043941 | 0.094093 | −4.14311 | CDH11 |
| 236300_at | −1.32375 | −2.50316 | 6.047608 | −2.105 | 0.04484 | 0.094093 | −4.14822 | PDE3A |
| 228128_x_at | −1.30545 | −2.47161 | 5.844653 | −2.86959 | 0.007937 | 0.089153 | −3.71417 | PAPPA |
| 227261_at | −1.28274 | −2.433 | 6.284268 | −2.52971 | 0.017632 | 0.089153 | −3.91283 | KLF12 |
| 226545_at | −1.26873 | −2.4095 | 5.894217 | −2.30297 | 0.029306 | 0.089153 | −4.04079 | CD109 |
| 222877_at | −1.25473 | −2.38623 | 5.51788 | −2.25731 | 0.032378 | 0.089153 | −4.06596 | NRP2 |
| 235408_x_at | −1.22278 | −2.33396 | 5.358524 | −2.53662 | 0.017356 | 0.089153 | −3.90886 | ZNF117 |
| 225381_at | −1.2194 | −2.32849 | 3.226568 | −2.13623 | 0.041981 | 0.094093 | −4.13159 | MIR100HG |
| 223278_at | −1.18872 | −2.2795 | 9.762843 | −2.39016 | 0.024166 | 0.089153 | −3.99213 | GJB2 |
| 228434_at | −1.14824 | −2.21643 | 4.287748 | −2.18882 | 0.037534 | 0.091859 | −4.1033 | BTNL9 |
| 230430_at | −1.1394 | −2.2029 | 6.144648 | −2.49906 | 0.018909 | 0.089153 | −3.93038 | ENTPD2 |
| 225450_at | −1.13376 | −2.19431 | 5.619859 | −2.34718 | 0.026587 | 0.089153 | −4.01621 | AMOTL1 |
| 230830_at | −1.12483 | −2.18076 | 3.760866 | −2.15888 | 0.040011 | 0.093025 | −4.11944 | SLC51B |
| 224252_s_at | −1.06815 | −2.09675 | 9.279824 | −2.74626 | 0.010653 | 0.089153 | −3.78697 | FXYD5 |
| 228742_at | −1.05836 | −2.08257 | 4.0007 | −2.24696 | 0.033113 | 0.089153 | −4.07164 | LOC101928092 |
| 242814_at | −1.05481 | −2.07744 | 6.387625 | −2.23877 | 0.033706 | 0.089153 | −4.07612 | SERPINB9 |
| 228176_at | −1.04444 | −2.06257 | 7.799295 | −2.23298 | 0.034131 | 0.089153 | −4.07928 | S1PR3 |
| 222925_at | −1.03259 | −2.0457 | 3.693013 | −2.01851 | 0.053682 | 0.101886 | −4.19363 | DCDC2 |
| 225990_at | −1.02379 | −2.03325 | 5.279346 | −2.08726 | 0.04654 | 0.094093 | −4.15762 | BOC |
| 225651_at | −1.02353 | −2.03289 | 6.012408 | −2.23828 | 0.033742 | 0.089153 | −4.07639 | UBE2E2 |
| 227189_at | 1.00737 | 2.010243 | 4.168682 | 2.479171 | 0.019782 | 0.089153 | −3.94173 | CPNE5 |
| 244084_at | 1.039848 | 2.056011 | 5.792369 | 2.215154 | 0.03547 | 0.089153 | −4.089 | AIFM3 |
| 231769_at | 1.040773 | 2.05733 | 6.140114 | 2.051865 | 0.050104 | 0.099143 | −4.17624 | FBXO6 |
| 233059_at | 1.080904 | 2.115361 | 2.989412 | 2.274979 | 0.031156 | 0.089153 | −4.05625 | KCNJ3 |
| 227803_at | 1.100636 | 2.144492 | 7.110862 | 2.223687 | 0.034823 | 0.089153 | −4.08436 | ENPP5 |
| 242283_at | 1.133123 | 2.19333 | 6.234929 | 2.528213 | 0.017692 | 0.089153 | −3.91368 | DNAH14 |
| 223275_at | 1.14486 | 2.211246 | 7.919203 | 2.334733 | 0.027328 | 0.089153 | −4.02315 | PRMT6 |
| 226269_at | 1.145197 | 2.211763 | 5.737558 | 2.033055 | 0.052095 | 0.100935 | −4.18607 | GDAP1 |
| 229812_at | 1.241509 | 2.364457 | 6.468448 | 3.199739 | 0.003531 | 0.065671 | −3.51699 | USP48 |
| 231192_at | 1.249695 | 2.377911 | 3.345659 | 2.622064 | 0.014252 | 0.089153 | −3.85951 | LPAR3 |
| 222838_at | 1.257912 | 2.391494 | 4.987706 | 2.371338 | 0.0252 | 0.089153 | −4.0027 | SLAMF7 |
| 228144_at | 1.260287 | 2.395434 | 3.302716 | 2.649899 | 0.013358 | 0.089153 | −3.84333 | ZNF300 |
| 230563_at | 1.316779 | 2.491093 | 2.996929 | 2.272681 | 0.031313 | 0.089153 | −4.05751 | RASGEF1A |
| 225767_at | 1.473927 | 2.777769 | 9.921784 | 2.094735 | 0.045817 | 0.094093 | −4.15366 | RNA45SN5 |
| 228988_at | 1.598321 | 3.027907 | 5.208193 | 3.535925 | 0.001506 | 0.065671 | −3.31553 | ZNF711 |
| 226926_at | 1.768904 | 3.407949 | 6.126031 | 2.404571 | 0.023401 | 0.089153 | −3.98402 | DMKN |
| 230774_at | 1.780767 | 3.436088 | 4.634571 | 5.101327 | 2.39E−05 | 0.002222 | −2.43946 | PTGR2 |
| 239697_x_at | 1.987583 | 3.96572 | 3.538509 | 2.583161 | 0.015595 | 0.089153 | −3.88204 | CFAP20DC |

### REFERENCES

ADDIN EN.REFLIST [1] Sung H, Ferlay J, Siegel R L, Laversanne M, Soerjomataram I, Jemal A, et al. Global Cancer Statistics 2020: GLOBOCAN Estimates of Incidence and Mortality Worldwide for 36 Cancers in 185 Countries. CA Cancer J Clin. 2021; 71:209-49.
[2] Araghi M, Arnold M, Rutherford M J, Guren M G, Cabasag C J, Bardot A, et al. Colon and rectal cancer survival in seven high-income countries 2010-2014: variation by age and stage at diagnosis (the ICBP SURVMARK-2 project). Gut. 2021; 70:114-26.
[3] Salonga D, Danenberg K D, Johnson M, Metzger R, Groshen S, Tsao-Wei D D, et al. Colorectal tumors responding to 5-fluorouracil have low gene expression levels of dihydropyrimidine dehydrogenase, thymidylate synthase, and thymidine phosphorylase. Clin Cancer Res. 2000; 6:1322-7.
[4] Showalter S L, Showalter T N, Witkiewicz A, Havens R, Kennedy E P, Hucl T, et al. Evaluating the drug-target relationship between thymidylate synthase expression and tumor response to 5-fluorouracil. Is it time to move forward?Cancer Biol Ther. 2008; 7:986-94.
[5] Su Y, Tian X, Gao R, Guo W, Chen C, Chen C, et al. Colon cancer diagnosis and staging classification based on machine learning and bioinformatics analysis. Computers in Biology and Medicine. 2022; 145:105409.
[6] Nguyen H T, Duong H Q. The molecular characteristics of colorectal cancer: Implications for diagnosis and therapy (Review). Oncol Lett. 2018; 16:9-18.
[7] Pelley R J. Oxaliplatin: a new agent for colorectal cancer. Curr Oncol Rep. 2001; 3:147-55.
[8] Douillard J Y, Cunningham D, Roth A D, Navarro M, James R D, Karasek P, et al. Irinotecan combined with fluorouracil compared with fluorouracil alone as first-line treatment for metastatic colorectal cancer: a multicentre randomised trial. Lancet. 2000; 355:1041-7.
[9] Hammond W A, Swaika A, Mody K. Pharmacologic resistance in colorectal cancer: a review. Therapeutic advances in medical oncology. 2016; 8:57-84.
[10] Bailly C. Irinotecan: 25 years of cancer treatment. Pharmacol Res. 2019; 148:104398.
[11] Alcindor T, Beauger N. Oxaliplatin: a review in the era of molecularly targeted therapy. Curr Oncol. 2011; 18:18-25.
[12] Dallas N A, Xia L, Fan F, Gray M J, Gaur P, van Buren G, 2nd, et al. Chemoresistant colorectal cancer cells, the cancer stem cell phenotype, and increased sensitivity to insulin-like growth factor-I receptor inhibition. Cancer Res. 2009; 69:1951-7.
[13] Mansoori B, Mohammadi A, Davudian S, Shirjang S, Baradaran B. The Different Mechanisms of Cancer Drug Resistance: A Brief Review. Adv Pharm Bull. 2017; 7:339-48.
[14] Tournigand C, Andre T, Achille E, Lledo G, Flesh M, Mery-Mignard D, et al. FOLFIRI followed by FOLFOX6 or the reverse sequence in advanced colorectal cancer: a randomized GERCOR study. J Clin Oncol. 2004; 22:229-37.
[15] Colucci G, Gebbia V, Paoletti G, Giuliani F, Caruso M, Gebbia N, et al. Phase III randomized trial of FOLFIRI versus FOLFOX4 in the treatment of advanced colorectal cancer: a multicenter study of the Gruppo Oncologico Dell'Italia Meridionale. J Clin Oncol. 2005; 23:4866-75.
[16] Frohlich H, Balling R, Beerenwinkel N, Kohlbacher O, Kumar S, Lengauer T, et al. From hype to reality: data science enabling personalized medicine. BMC Med. 2018; 16:150.
[17] Perez-Gracia J L, Sanmamed M F, Bosch A, Patino-Garcia A, Schalper K A, Segura V, et al. Strategies to design clinical studies to identify predictive biomarkers in cancer research. Cancer Treat Rev. 2017; 53:79-97.
[18] Lu W, Fu D, Kong X, Huang Z, Hwang M, Zhu Y, et al. FOLFOX treatment response prediction in metastatic or recurrent colorectal cancer patients via machine learning algorithms. Cancer Med. 2020; 9:1419-29.
[19] He J, Cheng J, Guan Q, Yan H, Li Y, Zhao W, et al. Qualitative transcriptional signature for predicting pathological response of colorectal cancer to FOLFOX therapy. Cancer science. 2020; 111:253-65.
[20] Tsuji S, Midorikawa Y, Takahashi T, Yagi K, Takayama T, Yoshida K, et al. Potential responders to FOLFOX therapy for colorectal cancer by Random Forests analysis. Br J Cancer. 2012; 106:126-32.
[21] Chan H C, Chattopadhyay A, Chuang E Y, Lu T P. Development of a Gene-Based Prediction Model for Recurrence of Colorectal Cancer Using an Ensemble Learning Algorithm. Frontiers in oncology. 2021; 11:631056.
[22] Cherradi S, Martineau P, Gongora C, Del Rio M. Claudin gene expression profiles and clinical value in colorectal tumors classified according to their molecular subtype. Cancer Manag Res. 2019; 11:1337-48.
[23] Cherradi S, Ayrolles-Torro A, Vezzo-Vie N, Gueguinou N, Denis V, Combes E, et al. Antibody targeting of claudin-1 as a potential colorectal cancer therapy. J Exp Clin Cancer Res. 2017; 36:89.
[24] Del Rio M, Molina F, Bascoul-Mollevi C, Copois V, Bibeau F, Chalbos P, et al. Gene expression signature in advanced colorectal cancer patients select drugs and response for the use of leucovorin, fluorouracil, and irinotecan. J Clin Oncol. 2007; 25:773-80.
[25] Del Rio M, Mollevi C, Bibeau F, Vie N, Selves J, Emile J F, et al. Molecular subtypes of metastatic colorectal cancer are associated with patient response to irinotecan-based therapies. Eur J Cancer. 2017; 76:68-75.
[26] Del Rio M, Mollevi C, Vezzio-Vie N, Bibeau F, Ychou M, Martineau P. Specific extracellular matrix remodeling signature of colon hepatic metastases. PLoS One. 2013; 8:e74599.
[27] Gharaibeh R Z, Fodor A A, Gibas C J. Background correction using dinucleotide affinities improves the performance of GCRMA. BMC Bioinformatics. 2008; 9:452.
[28] Irizarry R A, Bolstad B M, Collin F, Cope L M, Hobbs B, Speed T P. Summaries of Affymetrix GeneChip probe level data. Nucleic Acids Res. 2003; 31:e15.
[29] Irizarry R A, Warren D, Spencer F, Kim I F, Biswal S, Frank B C, et al. Multiple-laboratory comparison of microarray platforms. Nat Methods. 2005; 2:345-50.
[30] Ritchie M E, Phipson B, Wu D, Hu Y, Law C W, Shi W, et al. limma powers differential expression analyses for RNA-sequencing and microarray studies. Nucleic Acids Res. 2015; 43:e47.
[31] Tilford C A, Siemers N O. Gene set enrichment analysis. Methods Mol Biol. 2009; 563:99-121.
[32] Kauffmann A, Gentleman R, Huber W. arrayQualityMetrics—a bioconductor package for quality assessment of microarray data. Bioinformatics. 2009; 25:415-6.
[33] Kauffmann A, Huber W. Microarray data quality control improves the detection of differentially expressed genes. Genomics. 2010; 95:138-42.
[34] Tweedie S, Braschi B, Gray K, Jones T E, Seal R L, Yates B, et al. Genenames. org: the HGNC and VGNC resources in 2021. Nucleic acids research. 2021; 49:D939-D46.
[35] Braschi B, Seal R L, Tweedie S, Jones T E, Bruford E A. The risks of using unapproved gene symbols. The American Journal of Human Genetics. 2021; 108:1813-6.
[36] Carlson M R, Pages H, Arora S, Obenchain V, Morgan M. Genomic Annotation Resources in R/Bioconductor. Methods Mol Biol. 2016; 1418:67-90.
[37] Cheadle C, Vawter M P, Freed W J, Becker K G. Analysis of microarray data using Z score transformation. The Journal of molecular diagnostics. 2003; 5:73-81.
[38] Yasrebi H. Comparative study of joint analysis of microarray gene expression data in survival prediction and risk assessment of breast cancer patients. Briefings in bioinformatics. 2016; 17:771-85.
[39] Diaz-Uriarte R. GeneSrF and varSelRF: a web-based tool and R package for gene selection and classification using random forest. BMC Bioinformatics. 2007; 8:328.
[40] Ghosh Roy G, Geard N, Verspoor K, He S. PoLoBag: Polynomial Lasso Bagging for signed gene regulatory network inference from expression data. Bioinformatics. 2021; 36:5187-93.
[41] Hua J. LAK: Lasso and K-Means Based Single-Cell RNA-Seq Data Clustering Analysis. IEEE Access. 2020; 8:129679-88.
[42] Friedman J, Hastie T, Tibshirani R. Regularization paths for generalized linear models via coordinate descent. Journal of statistical software. 2010; 33:1.
[43] Zhou G, Soufan O, Ewald J, Hancock R E W, Basu N, Xia J. NetworkAnalyst 3.0: a visual analytics platform for comprehensive gene expression profiling and meta-analysis. Nucleic Acids Res. 2019; 47:W234-W41.
[44] Orchard S, Kerrien S, Abbani S, Aranda B, Bhate J, Bidwell S, et al. Protein interaction data curation: the International Molecular Exchange (IMEx) consortium. Nat Methods. 2012; 9:345-50.
[45] Kanehisa M, Furumichi M, Tanabe M, Sato Y, Morishima K. KEGG: new perspectives on genomes, pathways, diseases and drugs. Nucleic Acids Res. 2017; 45:D353-D61.
[46] Tang Z, Kang B, Li C, Chen T, Zhang Z. GEPIA2: an enhanced web server for large-scale expression profiling and interactive analysis. Nucleic acids research. 2019; 47:W556-W60.
[47] Tomczak K, Czerwinska P, Wiznerowicz M. Review The Cancer Genome Atlas (TCGA): an immeasurable source of knowledge. Contemporary Oncology/Wspólczesna Onkologia. 2015; 2015:68-77.
[48] Lonsdale J, Thomas J, Salvatore M, Phillips R, Lo E, Shad S, et al. The genotype-tissue expression (GTEx) project. Nature genetics. 2013; 45:580-5.
[49] Giacchetti S, Perpoint B, Zidani R, Le Bail N, Faggiuolo R, Focan C, et al. Phase III multicenter randomized trial of oxaliplatin added to chronomodulated fluorouracil-leucovorin as first-line treatment of metastatic colorectal cancer. J Clin Oncol. 2000; 18:136-47.
[50] Neugut A I, Lin A, Raab G T, Hillyer G C, Keller D, O'Neil D S, et al. FOLFOX and FOLFIRI Use in Stage IV Colon Cancer: Analysis of SEER-Medicare Data. Clin Colorectal Cancer. 2019; 18:133-40.
[51] Goldberg R M. Therapy for metastatic colorectal cancer. Oncologist. 2006; 11:981-7.
[52] Hess K R, Anderson K, Symmans W F, Valero V, Ibrahim N, Mejia J A, et al. Pharmacogenomic predictor of sensitivity to preoperative chemotherapy with paclitaxel and fluorouracil, doxorubicin, and cyclophosphamide in breast cancer. J Clin Oncol. 2006; 24:4236-44.
[53] Gordon G J, Jensen R V, Hsiao L L, Gullans S R, Blumenstock J E, Richards W G, et al. Using gene expression ratios to predict outcome among patients with mesothelioma. J Natl Cancer Inst. 2003; 95:598-605.
[54] Nutt C L, Mani D R, Betensky R A, Tamayo P, Cairncross J G, Ladd C, et al. Gene expression-based classification of malignant gliomas correlates better with survival than histological classification. Cancer Res. 2003; 63:1602-7.
[55] Parissenti A M, Hembruff S L, Villeneuve D J, Veitch Z, Guo B, Eng J. Gene expression profiles as biomarkers for the prediction of chemotherapy drug response in human tumour cells. Anticancer Drugs. 2007; 18:499-523.
[56] Nannini M, Pantaleo M A, Maleddu A, Astolfi A, Formica S, Biasco G. Gene expression profiling in colorectal cancer using microarray technologies: results and perspectives. Cancer Treat Rev. 2009; 35:201-9.
[57] Covic M, Hassa P O, Saccani S, Buerki C, Meier N I, Lombardi C, et al. Arginine methyltransferase CARM1 is a promoter-specific regulator of NF-kappaB-dependent gene expression. Embo j. 2005; 24:85-96.
[58] El Messaoudi S, Fabbrizio E, Rodriguez C, Chuchana P, Fauquier L, Cheng D, et al. Coactivator-associated arginine methyltransferase 1 (CARM1) is a positive regulator of the Cyclin E1 gene. Proc Natl Acad Sci USA. 2006; 103:13351-6.
[59] Valletti A, Marzano F, Pesole G, Sbisa E, Tullo A. Targeting Chemoresistant Tumors: Could TRIM Proteins-p53 Axis Be a Possible Answer?Int J Mol Sci. 2019; 20.
[60] Hong H, Kao C, Jeng M H, Eble J N, Koch M O, Gardner T A, et al. Aberrant expression of CARM1, a transcriptional coactivator of androgen receptor, in the development of prostate carcinoma and androgen-independent status. Cancer. 2004; 101:83-9.
[61] Frietze S, Lupien M, Silver P A, Brown M. CARM1 regulates estrogen-stimulated breast cancer growth through up-regulation of E2F1. Cancer Res. 2008; 68:301-6.
[62] Chen M, Sinha M, Luxon B A, Bresnick A R, O'Connor K L. Integrin alpha6beta4 controls the expression of genes associated with cell motility, invasion, and metastasis, including S100A4/metastasin. J Biol Chem. 2009; 284:1484-94.
[63] Kim Y R, Lee B K, Park R Y, Nguyen N T, Bae J A, Kwon D D, et al. Differential CARM1 expression in prostate and colorectal cancers. BMC Cancer. 2010; 10:197.
[64] He T C, Sparks A B, Rago C, Hermeking H, Zawel L, da Costa L T, et al. Identification of c-MYC as a target of the APC pathway. Science. 1998; 281:1509-12.
[65] Stein U, Arlt F, Walther W, Smith J, Waldman T, Harris E D, et al. The metastasis-associated gene S100A4 is a novel target of beta-catenin/T-cell factor signaling in colon cancer. Gastroenterology. 2006; 131:1486-500.
[66] McClanahan T, Koseoglu S, Smith K, Grein J, Gustafson E, Black S, et al. Identification of overexpression of orphan G protein-coupled receptor GPR49 in human colon and ovarian primary tumors. Cancer Biol Ther. 2006; 5:419-26.
[67] Nelson W J, Nusse R. Convergence of Wnt, beta-catenin, and cadherin pathways. Science. 2004; 303:1483-7.
[68] Segditsas S, Tomlinson I. Colorectal cancer and genetic alterations in the Wnt pathway. Oncogene. 2006; 25:7531-7.
[69] Grivennikov S I. Inflammation and colorectal cancer: colitis-associated neoplasia. Semin Immunopathol. 2013; 35:229-44.
[70] Gonzalez C G, Akula S, Burleson M. The role of mediator subunit 12 in tumorigenesis and cancer therapeutics. Oncol Lett. 2022; 23:74.
[71] Huang S, Hözel M, Knijnenburg T, Schlicker A, Roepman P, McDermott U, et al. MED12 controls the response to multiple cancer drugs through regulation of TGF-β receptor signaling. Cell. 2012; 151:937-50.
[72] Wang L, Zeng H, Wang Q, Zhao Z, Boyer T G, Bian X, et al. MED12 methylation by CARM1 sensitizes human breast cancer cells to chemotherapy drugs. Sci Adv. 2015; 1:e1500463.
[73] Zhang S, O'Regan R, Xu W. The emerging role of mediator complex subunit 12 in tumorigenesis and response to chemotherapeutics. Cancer. 2020; 126:939-48.
[74] Piao M-Y, Cao H-L, He N-N, Xu M-Q, Dong W-X, Wang W-Q, et al. Potential role of TRIM3 as a novel tumour suppressor in colorectal cancer (CRC) development. Scandinavian Journal of Gastroenterology. 2016; 51:572-82.
[75] Liu Y, Raheja R, Yeh N, Ciznadija D, Pedraza A M, Ozawa T, et al. TRIM3, a tumor suppressor linked to regulation of p21Wafl/Cip1. Oncogene. 2014; 33:308-15.
[76] Song Y, Guo Q, Gao S, Hua K. Tripartite motif-containing protein 3 plays a role of tumor inhibitor in cervical cancer. Biochemical and Biophysical Research Communications. 2018; 498:686-92.
[77] Sanchez-Prieto R, Rojas J M, Taya Y, Gutkind J S. A Role for the p38 Mitogen-activated Protein Kinase Pathway in the Transcriptional Activation of p53 on Genotoxic Stress by Chemotherapeutic Agents1. Cancer Research. 2000; 60:2464-72.
[78] Stramucci L, Pranteda A, Bossi G. Insights of Crosstalk between p53 Protein and the MKK3/MKK6/p38 MAPK Signaling Pathway in Cancer. Cancers. 2018; 10:131.
[79] Pierrat B, Simonen M, Cueto M, Mestan J, Ferrigno P, Heim J. SH3GLB, a New Endophilin-Related Protein Family Featuring an SH3 Domain. Genomics. 2001; 71:222-34.
[80] Cuddeback S M, Yamaguchi H, Komatsu K, Miyashita T, Yamada M, Wu C, et al. Molecular cloning and characterization of Bif-1. A novel Src homology 3 domain-containing protein that associates with Bax. J Biol Chem. 2001; 276:20559-65.
[81] Takahashi Y, Karbowski M, Yamaguchi H, Kazi A, Wu J, Sebti S M, et al. Loss of Bif-1 Suppresses Bax/Bak Conformational Change and Mitochondrial Apoptosis. Molecular and Cellular Biology. 2005; 25:9369-82.
[82] Jansson A, Sun X-F. Bax Expression Decreases Significantly From Primary Tumor to Metastasis in Colorectal Cancer. Journal of Clinical Oncology. 2002; 20:811-6.
[83] Sturm I, Köhne C-H, Wolff G, Petrowsky H, Hillebrand T, Hauptmann S, et al. Analysis of the p53/BAX Pathway in Colorectal Cancer: Low B A X Is a Negative Prognostic Factor in Patients With Resected Liver Metastases. Journal of Clinical Oncology. 1999; 17:1364-.
[84] McKnight N C, Zhenyu Y. Beclin 1, an Essential Component and Master Regulator of PI3K-III in Health and Disease. Curr Pathobiol Rep. 2013; 1:231-8.
[85] Mahgoub E, Taneera J, Sulaiman N, Saber-Ayad M. The role of autophagy in colorectal cancer: Impact on pathogenesis and implications in therapy. Frontiers in Medicine. 2022; 9.
[86] Li B X, Li C Y, Peng R Q, Wu X J, Wang H Y, Wan D S, et al. The expression of beclin 1 is associated with favorable prognosis in stage IIIB colon cancers. Autophagy. 2009; 5:303-6.
[87] Park J M, Huang S, Wu T T, Foster N R, Sinicrope F A. Prognostic impact of Beclin 1, p62/sequestosome 1 and LC3 protein expression in colon carcinomas from patients receiving 5-fluorouracil as adjuvant chemotherapy. Cancer Biol Ther. 2013; 14:100-7.
[88] Teixeira C S S, Sousa S F. Current Status of the Use of Multifunctional Enzymes as AntiCancer Drug Targets. Pharmaceutics. 2022; 14:10.
[89] Jeong C H, Bode A M, Pugliese A, Cho Y Y, Kim H G, Shim J H, et al. [6]-Gingerol suppresses colon cancer growth by targeting leukotriene A4 hydrolase. Cancer Res. 2009; 69:5584-91.
[90] Ihara A, Wada K, Yoneda M, Fujisawa N, Takahashi H, Nakajima A. Blockade of Leukotriene B4 Signaling Pathway Induces Apoptosis and Suppresses Cell Proliferation in Colon Cancer. Journal of Pharmacological Sciences. 2007; 103:24-32.
[91] Oi N, Yamamoto H, Langfald A, Bai R, Lee M-H, Bode A M, et al. LTA4H regulates cell cycle and skin carcinogenesis. Carcinogenesis. 2017; 38:728-37.
[92] Polyak K, Kato J Y, Solomon M J, Sherr C J, Massague J, Roberts J M, et al. p27Kip1, a cyclin-Cdk inhibitor, links transforming growth factor-beta and contact inhibition to cell cycle arrest. Genes Dev. 1994; 8:9-22.
[93] Shapira M, Ben-Izhak O, Linn S, Futerman B, Minkov I, Hershko D D. The prognostic impact of the ubiquitin ligase subunits Skp2 and Cks1 in colorectal carcinoma. Cancer. 2005; 103:1336-46.
[94] Wang J R, Gan W J, Li X M, Zhao Y Y, Li Y, Lu X X, et al. Orphan nuclear receptor Nur77 promotes colorectal cancer invasion and metastasis by regulating MMP-9 and E-cadherin. Carcinogenesis. 2014; 35:2474-84.
[95] Hedrick E, Lee S O, Safe S. The nuclear orphan receptor NR4A1 regulates β1-integrin expression in pancreatic and colon cancer cells and can be targeted by NR4A1 antagonists. Mol Carcinog. 2017; 56:2066-75.
[96] To S K, Zeng W J, Zeng J Z, Wong A S. Hypoxia triggers a Nur77-β-catenin feed-forward loop to promote the invasive growth of colon cancer cells. Br J Cancer. 2014; 110:935-45.
[97] Lee S O, Li X, Khan S, Safe S. Targeting N R4A1 (TR3) in cancer cells and tumors. Expert Opin Ther Targets. 2011; 15:195-206.
[98] Guengerich F P, Shimada T. Activation of procarcinogens by human cytochrome P450 enzymes. Mutat Res. 1998; 400:201-13.
[99] Kumarakulasingham M, Rooney P H, Dundas S R, Telfer C, Melvin W T, Curran S, et al. Cytochrome p450 profile of colorectal cancer: identification of markers of prognosis. Clin Cancer Res. 2005; 11:3758-65.
[100] Windmill K F, McKinnon R A, Zhu X, Gaedigk A, Grant D M, McManus M E. The role of xenobiotic metabolizing enzymes in arylamine toxicity and carcinogenesis: functional and localization studies. Mutat Res. 1997; 376:153-60.
[101] Hedrich W D, Hassan H E, Wang H. Insights into CYP2B6-mediated drug-drug interactions. Acta Pharm Sin B. 2016; 6:413-25.
[102] Nilius B, Prenen J, Janssens A, Owsianik G, Wang C, Zhu M X, et al. The selectivity filter of the cation channel TRPM4. J Biol Chem. 2005; 280:22899-906.
[103] Sagredo A I, Sagredo E A, Pola V, Echeverria C, Andaur R, Michea L, et al. TRPM4 channel is involved in regulating epithelial to mesenchymal transition, migration, and invasion of prostate cancer cell lines. J Cell Physiol. 2019; 234:2037-50.
[104] Holzmann C, Kappel S, Kilch T, Jochum M M, Urban S K, Jung V, et al. Transient receptor potential melastatin 4 channel contributes to migration of androgen-insensitive prostate cancer cells. Oncotarget. 2015; 6:41783-93.
[105] Fearon E R. Molecular genetics of colorectal cancer. Annual Review of Pathology: Mechanisms of Disease. 2011; 6:479-507.
[106] Kappel S, Stoklosa P, Hauert B, Ross-Kaschitza D, Borgström A, Baur R, et al. TRPM4 is highly expressed in human colorectal tumor buds and contributes to proliferation, cell cycle, and invasion of colorectal cancer cells. Mol Oncol. 2019; 13:2393-405.
[107] Björnsson J M, Andersson E, Lundström P, Larsson N, Xu X, Repetowska E, et al. Proliferation of primitive myeloid progenitors can be reversibly induced by HOXA10. Blood. 2001; 98:3301-8.
[108] Ordóñez-Morán P, Dafflon C, Imajo M, Nishida E, Huelsken J. HOXA5 Counteracts Stem Cell Traits by Inhibiting Wnt Signaling in Colorectal Cancer. Cancer Cell. 2015; 28:815-29.
[109] Li T, Xu C, Cai B, Zhang M, Gao F, Gan J. Expression and clinicopathological significance of the lncRNA HOXA11-AS in colorectal cancer. Oncol Lett. 2016; 12:4155-60.
[110] Li W, Jia G, Qu Y, Du Q, Liu B, Liu B. Long Non-Coding RNA (LncRNA) HOXA11-AS Promotes Breast Cancer Invasion and Metastasis by Regulating Epithelial-Mesenchymal Transition. Med Sci Monit. 2017; 23:3393-403.
[111] Liu Z, Chen Z, Fan R, Jiang B, Chen X, Chen Q, et al. Over-expressed long noncoding RNA HOXA11-AS promotes cell cycle progression and metastasis in gastric cancer. Mol Cancer. 2017; 16:82.
[112] Kim H J, Eoh K J, Kim L K, Nam E J, Yoon S O, Kim K H, et al. The long noncoding RNA HOXA11 antisense induces tumor progression and stemness maintenance in cervical cancer. Oncotarget. 2016; 7:83001-16.
[113] Chen D, Sun Q, Zhang L, Zhou X, Cheng X, Zhou D, et al. The lncRNA HOXA11-AS functions as a competing endogenous RNA to regulate PADI2 expression by sponging miR-125a-5p in liver metastasis of colorectal cancer. Oncotarget. 2017; 8:70642-52.
[114] Smith J M, Hedman A C, Sacks D B. IQGAPs choreograph cellular signaling from the membrane to the nucleus. Trends Cell Biol. 2015; 25:171-84.
[115] Briggs M W, Sacks D B. IQGAP proteins are integral components of cytoskeletal regulation. EMBO Rep. 2003; 4:571-4.
[116] Nabeshima K, Shimao Y, Inoue T, Koono M. Immunohistochemical analysis of IQGAP1 expression in human colorectal carcinomas: its overexpression in carcinomas and association with invasion fronts. Cancer Lett. 2002; 176:101-9.
[117] Hayashi H, Nabeshima K, Aoki M, Hamasaki M, Enatsu S, Yamauchi Y, et al. Overexpression of IQGAP1 in advanced colorectal cancer correlates with poor prognosis-critical role in tumor invasion. Int J Cancer. 2010; 126:2563-74.
[118] Zhang Z, Wei Y, Li X, Zhao R, Wang X, Yang Z, et al. IQGAP1 enhances cell invasion and matrix metalloproteinase-2 expression through upregulating NF-κB activity in esophageal squamous cell carcinoma cells. Gene. 2022; 824:146406.
[119] Walch A, Seidl S, Hermannstadter C, Rauser S, Deplazes J, Langer R, et al. Combined analysis of Rac1, IQGAP1, Tiaml and E-cadherin expression in gastric cancer. Mod Pathol. 2008; 21:544-52.
[120] Wu Z, Irizarry R A, Gentleman R, Martinez-Murillo F, Spencer F. A Model-Based Background Adjustment for Oligonucleotide Expression Arrays. Journal of the American Statistical Association. 2004; 99:909-17.
[121] Wu C C, Li H, Xiao Y, Yang L L, Chen L, Deng W W, et al. Over-expression of IQGAP1 indicates poor prognosis in head and neck squamous cell carcinoma. J Mol Histol. 2018; 49:389-98.
[122] Jadeski L, Mataraza J M, Jeong H W, Li Z, Sacks D B. IQGAP1 stimulates proliferation and enhances tumorigenesis of human breast epithelial cells. J Biol Chem. 2008; 283:1008-17.
[123] Kumar D, Hassan M K, Pattnaik N, Mohapatra N, Dixit M. Reduced expression of IQGAP2 and higher expression of IQGAP3 correlates with poor prognosis in cancers. PLoS One. 2017; 12:e0186977.
[124] Pelossof R, Chow O S, Fairchild L, Smith J J, Setty M, Chen C T, et al. Integrated genomic profiling identifies microRNA-92a regulation of IQGAP2 in locally advanced rectal cancer. Genes Chromosomes Cancer. 2016; 55:311-21.
[125] He P Y, Yip W K, Chai B L, Chai B Y, Jabar M F, Dusa N, et al. Inhibition of cell migration and invasion by miR-29a-3p in a colorectal cancer cell line through suppression of CDC42BPA mRNA expression. Oncol Rep. 2017; 38:3554-66.
[126] Deng L, Jiang N, Zeng J, Wang Y, Cui H. The Versatile Roles of Cancer-Associated Fibroblasts in Colorectal Cancer and Therapeutic Implications. Front Cell Dev Biol. 2021; 9:733270.
[127] Schmidt V A, Chiariello C S, Capilla E, Miller F, Bahou W F. Development of hepatocellular carcinoma in Iqgap2-deficient mice is IQGAP1 dependent. Mol Cell Biol. 2008; 28:1489-502.
[128] Kaitsuka T, Matsushita M. Regulation of translation factor EEF1D gene function by alternative splicing. Int J Mol Sci. 2015; 16:3970-9.
[129] Ong L L, Er C P, Ho A, Aung M T, Yu H. Kinectin anchors the translation elongation factor-1 delta to the endoplasmic reticulum. J Biol Chem. 2003; 278:32115-23.
[130] Shi L, Campbell G, Jones W D, Campagne F, Wen Z, Walker S J, et al. The MicroArray Quality Control (MAQC)-II study of common practices for the development and validation of microarray-based predictive models. Nat Biotechnol. 2010; 28:827-38.
[131] Ogawa K, Utsunomiya T, Mimori K, Tanaka Y, Tanaka F, Inoue H, et al. Clinical significance of elongation factor-1 delta mRNA expression in oesophageal carcinoma. Br J Cancer. 2004; 91:282-6.
[132] Xu H, Yu S, Peng K, Gao L, Chen S, Shen Z, et al. The role of EEF1D in disease pathogenesis: a narrative review. Ann Transl Med. 2021; 9:1600.
[133] Drury J, Rychahou P G, Kelson C O, Geisen M E, Wu Y, He D, et al. Upregulation of CD36, a Fatty Acid Translocase, Promotes Colorectal Cancer Metastasis by Increasing MMP28 and Decreasing E-Cadherin Expression. Cancers (Basel). 2022; 14.
[134] Drury J, Rychahou P G, Kelson C O, Geisen M E, Wu Y, He D, et al. Upregulation of CD36, a Fatty Acid Translocase, Promotes Colorectal Cancer Metastasis by Increasing MMP28 and Decreasing E-Cadherin Expression. Cancers. 2022; 14:252.
[135] Yu Y, Liu D, Liu Z, Li S, Ge Y, Sun W, et al. The inhibitory effects of COL1A2 on colorectal cancer cell proliferation, migration, and invasion. J Cancer. 2018; 9:2953-62.
[136] Kalmár A, Péterfia B, Hollósi P, Galamb O, Spisik S, Wichmann B, et al. DNA hypermethylation and decreased mRNA expression of MAL, PRIMAl, PTGDR and SFRP1 in colorectal adenoma and cancer. BMC Cancer. 2015; 15:736.
[137] Sengupta P K, Smith E M, Kim K, Murnane M J, Smith B D. DNA hypermethylation near the transcription start site of collagen alpha2(I) gene occurs in both cancer cell lines and primary colorectal cancers. Cancer Res. 2003; 63:1789-97.
[138] Turashvili G, Bouchal J, Baumforth K, Wei W, Dziechciarkova M, Ehrmann J, et al. Novel markers for differentiation of lobular and ductal invasive breast carcinomas by laser microdissection and microarray analysis. BMC Cancer. 2007; 7:55.
[139] Tilman G, Mattiussi M, Brasseur F, van Baren N, Decottignies A. Human periostin gene expression in normal tissues, tumors and melanoma: evidences for periostin production by both stromal and melanoma cells. Mol Cancer. 2007; 6:80.
[140] Paper W, Kroeber M, Heersink S, Stephan D A, Fuchshofer R, Russell P, et al. Elevated amounts of myocilin in the aqueous humor of transgenic mice cause significant changes in ocular gene expression. Exp Eye Res. 2008; 87:257-67.
[141] Pham V C, Henzel W J, Lill J R. Rapid on-membrane proteolytic cleavage for Edman sequencing and mass spectrometric identification of proteins. Electrophoresis. 2005; 26:4243-51.
[142] Hu T, Wu X, Li K, Li Y, He P, Wu Z, et al. AKAP12 Endogenous Transcripts Suppress The Proliferation, Migration And Invasion Of Colorectal Cancer Cells By Directly Targeting oncomiR-183-5p. Onco Targets Ther. 2019; 12:8301-10.
[143] He P, Li K, Li S B, Hu T T, Guan M, Sun F Y, et al. Upregulation of AKAP12 with HDAC3 depletion suppresses the progression and migration of colorectal cancer. Int J Oncol. 2018; 52:1305-16.
[144] Troyanovsky B, Levchenko T, Minsson G, Matvijenko O, Holmgren L. Angiomotin: an angiostatin binding protein that regulates endothelial cell migration and tube formation. J Cell Biol. 2001; 152:1247-54.
[145] Bratt A, Birot O, Sinha I, Veitonmaki N, Aase K, Ernkvist M, et al. Angiomotin regulates endothelial cell-cell junctions and cell motility. J Biol Chem. 2005; 280:34859-69.
[146] Zhang Y, Yuan J, Zhang X, Yan F, Huang M, Wang T, et al. Angiomotin promotes the malignant potential of colon cancer cells by activating the YAP-ERK/PI3K-AKT signaling pathway. Oncol Rep. 2016; 36:3619-26.
[147] Kojima Y, Kondo Y, Fujishita T, Mishiro-Sato E, Kajino-Sakamoto R, Taketo M M, et al. Stromal iodothyronine deiodinase 2 (DIO2) promotes the growth of intestinal tumors in Apc(Δ716) mutant mice. Cancer Sci. 2019; 110:2520-8.
[148] St Croix B, Rago C, Velculescu V, Traverso G, Romans K E, Montgomery E, et al. Genes expressed in human tumor endothelium. Science. 2000; 289:1197-202.
[149] Dentice M, Luongo C, Ambrosio R, Sibilio A, Casillo A, Iaccarino A, et al. β-Catenin regulates deiodinase levels and thyroid hormone signaling in colon cancer cells. Gastroenterology. 2012; 143:1037-47.
[150] Kress E, Rezza A, Nadjar J, Samarut J, Plateroti M. The frizzled-related sFRP2 gene is a target of thyroid hormone receptor alphal and activates beta-catenin signaling in mouse intestine. J Biol Chem. 2009; 284:1234-41.
[151] Plateroti M, Kress E, Mori J I, Samarut J. Thyroid hormone receptor alphal directly controls transcription of the beta-catenin gene in intestinal epithelial cells. Mol Cell Biol. 2006; 26:3204-14.
[152] Rostkowska O, Olejniczak-Keder A, Spychalski P, Szarynska M, Kobiela J. Triiodothyronine lowers the potential of colorectal cancer stem cells <em>in vitro</em>. Oncol Rep. 2023; 49:21.
[153] Nasir A, Helm J, Turner L, Chen D T, Strosberg J, Hafez N, et al. RUNX1T1: a novel predictor of liver metastasis in primary pancreatic endocrine neoplasms. Pancreas. 2011; 40:627-33.
[154] Tonks A, Pearn L, Mills K I, Burnett A K, Darley R L. The sensitivity of human cells expressing RUNX1-RUNX1T1 to chemotherapeutic agents. Leukemia. 2006; 20:1883-5.
[155] Musaad A, Radhakrishnan V, Nehad M A. Runt-related Transcription Factor 1 (&lt;em&gt;RUNX1T1&lt;/em&gt;) Suppresses Colorectal Cancer Cells Through Regulation of Cell Proliferation and Chemotherapeutic Drug Resistance. Anticancer Research. 2016; 36:5257.
[156] Ghanem A, Schweitzer K, Naumann M. Catalytic domain of deubiquitinylase USP48 directs interaction with Rel homology domain of nuclear factor kappaB transcription factor RelA. Mol Biol Rep. 2019; 46:1369-75.
[157] Schweitzer K, Naumann M. CSN-associated USP48 confers stability to nuclear NF-κB/RelA by trimming K48-linked Ub-chains. Biochim Biophys Acta. 2015; 1853:453-69.
[158] Li S, Wang D, Zhao J, Weathington N M, Shang D, Zhao Y. The deubiquitinating enzyme USP48 stabilizes TRAF2 and reduces E-cadherin-mediated adherens junctions. Faseb j. 2018; 32:230-42.
[159] Zhou A, Lin K, Zhang S, Ma L, Xue J, Morris S A, et al. Gli1-induced deubiquitinase USP48 aids glioblastoma tumorigenesis by stabilizing Gli1. EMBO Rep. 2017; 18:1318-30.
[160] Tanouchi A, Taniuchi K, Furihata M, Naganuma S, Dabanaka K, Kimura M, et al. CCDC88A, a prognostic factor for human pancreatic cancers, promotes the motility and invasiveness of pancreatic cancer cells. J Exp Clin Cancer Res. 2016; 35:190.
[161] Jin F, Liu C, Guo Y, Chen H, Wu Y. Clinical implications of Girdin and PI3K protein expression in breast cancer. Oncol Lett. 2013; 5:1549-53.
[162] Wang A, Wang J, Sun L, Jin J, Ren H, Yang F, et al. Expression of tumor necrosis factor receptor-associated factor 4 correlates with expression of Girdin and promotes nuclear translocation of Girdin in breast cancer. Mol Med Rep. 2015; 11:3635-41.
[163] Gromova P, Ralea S, Lefort A, Libert F, Rubin B P, Erneux C, et al. Kit K641E oncogene up-regulates Sprouty homolog 4 and trophoblast glycoprotein in interstitial cells of Cajal in a murine model of gastrointestinal stromal tumours. J Cell Mol Med. 2009; 13:1536-48.
[164] Tian X, Liu Z, Niu B, Zhang J, Tan T K, Lee S R, et al. E-cadherin/β-catenin complex and the epithelial barrier. J Biomed Biotechnol. 2011; 2011:567305.
[165] Gong J, Zhou Y, Liu D, Huo J. F-box proteins involved in cancer-associated drug resistance. Oncol Lett. 2018; 15:8891-900.
[166] Zhang Y W, Brognard J, Coughlin C, You Z, Dolled-Filhart M, Aslanian A, et al. The F box protein Fbx6 regulates Chk1 stability and cellular sensitivity to replication stress. Mol Cell. 2009; 35:442-53.
[167] Qian Y, Daza J, Itzel T, Betge J, Zhan T, Marmé F, et al. Prognostic Cancer Gene Expression Signatures: Current Status and Challenges. Cells. 2021; 10.
[168] Fang Z, Xu S, Xie Y, Yan W. Identification of a prognostic gene signature of colon cancer using integrated bioinformatics analysis. World Journal of Surgical Oncology. 2021; 19:13.
[169] Dalerba P, Sahoo D, Paik S, Guo X, Yothers G, Song N, et al. CDX2 as a Prognostic Biomarker in Stage II and Stage III Colon Cancer. New England Journal of Medicine. 2016; 374:211-22.
[170] Hansen T F, Kjor-Frifeldt S, Eriksen A C, Lindebjerg J, Jensen L H, Ssrensen F B, et al. Prognostic impact of CDX2 in stage II colon cancer: results from two nationwide cohorts. Br J Cancer. 2018; 119:1367-73.
[171] Zhang Q N, Zhu H L, Xia M T, Liao J, Huang X T, Xiao J W, et al. A panel of collagen genes are associated with prognosis of patients with gastric cancer and regulated by microRNA-29c-3p: an integrated bioinformatics analysis and experimental validation. Cancer Manag Res. 2019; 11:4757-72.
[172] Abraham J P, Magee D, Cremolini C, Antoniotti C, Halbert D D, Xiu J, et al. Clinical Validation of a Machine-learning-derived Signature Predictive of Outcomes from First-line Oxaliplatin-based Chemotherapy in Advanced Colorectal Cancer. Clinical Cancer Research. 2021; 27:1174-83.
[173] Sharma A, Dey P. A machine learning approach to unmask novel gene signatures and prediction of Alzheimer's disease within different brain regions. Genomics. 2021; 113:1778-89.

What is claimed:

1. A method for predicting the response of a colorectal cancer patient (CRC) patient to FOLFOX said method selected from the group consisting of:
(A) if the patient is at any stage of CRC
(i) determining in a sample isolated from said patient the expression levels of one or more biomarkers, or their expression product, wherein the biomarker is the transcript, or protein product, of one or more genes selected from the group consisting of: GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKRIC1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2 and EPHB3; and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment;
(B) if the patient is at early stage of CRC
(i) determining in a sample isolated from said patient the expression levels of one or more biomarkers, or their expression product, wherein the biomarker is the transcript, or protein product, of one or more genes selected from the group consisting of: PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2 and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment; and
(C) if the patient is at the metastatic stage of CRC
(i) determining in a sample isolated from said patient the expression levels of one or more biomarkers, or their expression product, in a patient sample, wherein the biomarker is the transcript, or protein product, of one or more genes selected from the group consisting of: EEF1D, HSD17B2, ADH1B, RPS10, CA2, RPS1,1 ADAMDEC1, AKR1B10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL13RA2, NME7, REEP1, TACSTD2, EDNRB, NR5A2, P2RY14, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI and
(ii) comparing the expression levels of said one or more genes obtained in step (i) with established expression levels for responders and/or non-responders wherein similarities in the established expression levels for either responders or non-responders is indicative of whether the patient will respond to FOLFOX chemotherapy treatment.

2. The method of claim 1, wherein the expression level of the one or more biomarker transcripts is determined through the use of a polymerase chain reaction.

3. The method of claim 1, wherein the expression level of the one or more biomarker transcripts is determined through the use of a probe that is complementary to and binds to the biomarker transcript.

4. The method of claim 1, wherein the expression level of the one or more biomarker protein products is determined through the use of an immunoassay.

5. The method of claim 1, wherein the expression level of the one or more biomarker transcript, or protein product, is determined through the use of a microarray.

6. The method of claim 1, wherein comparing the expression levels of said one or more genes obtained in one of: step (A)(1), step (B)(1), or step (C)(1) comprises:
computing a prediction score based on the respective expression levels of said one or more genes, the prediction score being a weighted sum of the respective expression levels.

7. The method of claim 1, wherein comparing the expression levels of said one or more genes obtained in one of: step (A)(1), step (B)(1), or step (C)(1) comprises:
processing the respective expression levels of said one or more genes by a trained machine learning model to provide an output indicative of responsiveness or non-responsiveness to FOLFOX chemotherapy treatment.

8. A method for predicting the response of a colorectal cancer patient (CRC) patient to FOLFIRI said method selected from the group consisting of:
(A) if the patient is at any stage of CRC
(i) determining in a sample isolated from said patient the expression levels of one or more biomarkers, or their expression product, in a patient sample, wherein the biomarker is the transcript, or protein product, of one or more genes selected from the group consisting of: AKAP12, SFRP2, CD36, PTGR2, PRKG1, SLIT2, FBXO32, S1PR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1; and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment;
(B) if the patient is at early stage of CRC
(i) determining in a sample isolated from said patient the expression levels of one or more biomarkers, or their expression product, in a patient sample, wherein the biomarker is the transcript, or protein product, of one or more genes selected from the group consisting of: CCN4, ABCC13, AKAP12, GLT8D2, LUM, CTHRC1, FLRT3, SFRP2, COL1A2, MIR100HG, and PCDH7 and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment; and
(C) if the patient is at metastatic stage of CRC
(i) determining in a sample isolated from said patient the expression levels of one or more biomarkers, or their expression product, in a patient sample, wherein the biomarker is the transcript, or protein product, of one or more genes selected from the group consisting of: PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBXO6 and
(ii) comparing the expression levels of said one or more genes obtained in step (i) with established expression levels for responders and/or non-responders wherein similarities in the established expression levels for either responders or non-responders is indicative of whether the patient will respond to FOLFIRI chemotherapy treatment.

9. The method of claim 8, wherein the expression level of the one or more biomarker transcripts is determined through the use of a polymerase chain reaction.

10. The method of claim 8, wherein the expression level of the one or more biomarker transcripts is determined through the use of a probe that is complementary to and binds to the biomarker transcript.

11. The method of claim 8, wherein the expression level of the one or more biomarker protein products is determined through the use of an immunoassay.

12. The method of claim 8, wherein the expression level of the one or more biomarker transcript, or protein product, is determined through the use of a microarray.

13. The method of claim 8, wherein comparing the expression levels of said one or more genes obtained in one of: step (A)(1), step (B)(1), or step (C)(1) comprises:
computing a prediction score based on the respective expression levels of said one or more genes, the prediction score being a weighted sum of the respective expression levels.

14. The method of claim 8, wherein comparing the expression levels of said one or more genes obtained in one of: step (A)(1), step (B)(1), or step (C)(1) comprises:
processing the respective expression levels of said one or more genes by a trained machine learning model to provide an output indicative of responsiveness or non-responsiveness to FOLFIRI chemotherapy treatment.

15. A microarray for predicting the response of a colorectal cancer patient (CRC) patient to FOLFIRI or FOLFOX said microarray comprising one or more probes corresponding a group of biomarkers selected from the group consisting of:
(i) GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2 and EPHB3; and
(ii) PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2;
(iii) EEF1D, HSD17B2, ADH1B, RPS10, CA2, RPS1,1 ADAMDEC1, AKR1B10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL13RA2, NME7, REEP1, TACSTD2, EDNRB, NR5A2, P2RY14, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI and (iv) AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1; and
(iv) CCN4, ABCC13, AKAP12, GLT8D2, LUM, CTHRC1, FLRT3, SFRP2, COL1A2, MIR100HG, and PCDH7; and
(vi) PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBXO6.

16. The microarray of claim 15, wherein the probe is nucleic acid molecule that is complementary to and binds to a biomarker transcript.

17. The microarray of claim 15, wherein the probe is an antibody, or fragment thereof, that binds to a biomarker protein product.

18. A test kit for predicting the response of a colorectal cancer patient (CRC) patient to FOLFIRI or FOLFOX comprising a group of one or more probes for measuring the expression level of one or more biomarkers said biomarkers selected from the groups consisting of:
(i) GRP, FGF9, TRNP1, PHACTR3, KCNN4, CARM1, LTA4H, GTF2A1, TRIM3, GPN3, HELZ2, PPDPF, SERPINA1, SFR1, SH3GLB1, MPP7, AKR1C1, IGFBP1, HCAR3, F5, FRMD5, RPS23, ARHGAP5, PEG10, ALDH1A1, ACADSB, PSMA5, NTN4, MPI, GDPD1, VTI1B, ST6GAL2 and EPHB3; and
(ii) PEG10, TRPM4, BEX2, TLR1, HOXA11, FZD3, TFAP2C, IGF1, GPR34, EPHB3, PRKAA2, FABP6, SGCE, NR4A1, FZD7, PFN2, MLF1, DAAM1, C2orf88, CPE, MYEF2, EDN3, DEFB1, PTPRR, HOXA10, DNAJC12, BNIP3, PHACTR3, ASPN, MFAP3L, FRMD5, LRRN1, PPBP, KRT23, GRM8, CYP2B6, CHRM3, and CKMT2;
(iii) EEF1D, HSD17B2, ADH1B, RPS10, CA2, RPS1,1 ADAMDEC1, AKR1B10, RAB3IP, DNASE1L3, C1QB, ZG16B, ZFP36L2, UCA1, CDKN1C, SLC2A10, RETNLB, CXCL5, CAB39L, PPAT, CLDN8, SCG5, BEX4, LTN1, CREB5, ITLN1, ABCA8, CCL28, FER1L4, CD177, IGF1, LPAR1, AGR2, MOGAT2, MT1H, EVI2B, GCG, CCL11, REG4, POU2AF1, ADTRP, SCNN1B HEPACAM2, PLA2G10, SAMSN1, GABRE, AXDND1, LRRC69, PPFIBP1, MUC5B, PLLP, MT1M, ANG, BMP, FAM107B, SIM2, ZG16, ABI3BP, ATP2A3, GZMA, MUC2, ST6GALNAC1, FCGBP, C2orf88, HSD17B6, USH1C S100A8, WT1, IGLJ3 BCAS1, B3GNT7, SPARCL, PLEC, CH25H, LGR5, MT1F, SLAMF7, ZNF300, FBXL6, RGS2, IL13RA2, NME7, REEP1, TACSTD2, EDNRB, NR5A2, P2RY14, RPL27A, CNTN3, PCK1, IGLV3-25, EPB41L3, LGALS2, CLCA1, RASSF10, FCGR3B, JCHAIN, IGKC, MS4A12, ASCL2, MLPH, CHGA, NT5E and SI and
(iv) AKAP12, SFRP2, CD36, PTGR2, PRKG1, SLIT2, FBXO32, SIPR3, DDR2, MAP1B, GLT8D2, NRP2, RNF183, AMOTL1, BOC, PI1, CLMP, MIR100HG, CAB39L, LEMD1, FNDC1, CDH11, ADAM12, and CTHRC1; and
(iv) CCN4, ABCC13, AKAP12, GLT8D2, LUM, CTHRC1, FLRT3, SFRP2, COL1A2, MIR100HG, and PCDH7; and
(vi) PTGR2, P115, RAB3IP, USP48, PCDHB16, DDR2, PAPPA, FXYD5, ZNF300, FBXO32, ZNF117, DNAH14, ENTPD2, GJB2, SLAM7, SFTA2, COL3A1, AMOTL1, PRMT6, SLIT2, CD109, KCNJ3, NRP2, SERPINB9, SIPR3, BTNL9, PCDH7, FNDC1, CDH11, PDE3A, BOC, GDAP1, and FBXO6.

19. The test kit of claim 18, wherein the probe is nucleic acid molecule that is complementary to and binds to a biomarker transcript.

20. The test kit of claim 18, wherein the probe is an antibody, or fragment thereof, that binds to a biomarker protein product.

21. The method of claim 1, further comprising the step of creating a report summarizing the data obtained by analysis of the expression levels of one or more biomarkers.

22. The method of claim 8, further comprising the step of creating a report summarizing the data obtained by analysis of the expression levels of one or more biomarkers.
